SEQUENCE LISTING (72 sequences)

SEQ ID NO: 1 - DNA, 387 bp, Artificial sequence
  Synthetic oligonucleotide
gaagtgcagctggtgcagtctgggggaggcttggtccagcctggggggtccctgagactctcttgttcagcctctgggttcacctttagaacctatgccatgagctgggtccgccaggctccagggaaggggctgcagtgggtctcagttattagtcgtagtggtgataccacctaccacacagactccgtgaagggccgattcaccatctccagagacaattccaggaacgcgctgtatctgcaattggacagcctgagagccgaggacacggccttatattactgtgcgaaaacaggtaatatggcagtaggtgaccgaaggacaaactactcctactactacatggacgtctggggcaaagggaccacggtcaccgtctcctca

SEQ ID NO: 2 - DNA, 321 bp, Artificial sequence
  Synthetic oligonucleotide
gatattgtgatgactcagtctccttccaccctgtctgcttctgtaggagacagagtcaccatcacttgccgggccagtcagagtattaagtactggttggcctggtatcagcagaaaccagggaaagcccctaagctcctgatctataaggcatctaatttggaaagtggggtcccatcaaggttcagcggcagtggatctgggacagaattcactctcaccatcagcagcctgcggcctgatgattttgcaacttattactgccaacagtataatagttaccccctcactttcggcggagggaccacggtggagatcaaa

SEQ ID NO: 3 - DNA, 354 bp, Artificial sequence
  Synthetic oligonucleotide
gaggtgcagctggtggagtctgggggaggcttggtaaagcctggggggtccctgagactctcctgtaaagcatctggattcaatttcactaactcctggatgagttgggtccgccaggctccagggaagggactggagtggctgggtcgtattaaaagtgagtctgatggtggggcaacacgctacgctgcacccgttacgggaaggttttccatctccagagatgattcaagagacatgctgtttctgcaaatgaacagtctgacaaccgacgacacagcgatgtattattgtactacaaataaggtgactacaaattattggggccagggaacgctggtcaccgtctcatca

SEQ ID NO: 4 - DNA, 339 bp, Artificial sequence
  Synthetic oligonucleotide
gacattgtgatgactcagtctccagtcaccctggctgtgtctctgggcgagagggccaccatcaactgcaagtccagccagagtcttttatacagctccgacaatgagaactacttaacttggtaccagcagaaaccaggacagcctcctaagttgctcatttactgggcgtctgtccgagaatccgggattcctgaccgattcattggcagcgggtctgtgacagatttcactctcaccatcaacaatgtgcaggctgaagatgtggcagtttattactgtcaacaatttcgctatactcctctgacttttggccaggggaccacgcttgagatcaaa

SEQ ID NO: 5 - DNA, 339 bp, Artificial sequence
  Synthetic oligonucleotide
gaggttcagctggtggagtctggggctgaggtgaagaggcctggggcctcagtgagggtctcctgcaaggcttctggatacagcttcaccctctactatatgcactgggtgcgacaggcccctggccaaggactcgagtggctgggatggatcaaccctaaaactggtgacgtcaaatatgcacagaagtttcagggcagggtctccttgaccagggatacgagaatgaacacagcctacttggacttgacgaggctgagatctgacgacacggcccgctactactgtttgagggcttttgatctgtggggccgagggacaatgatcatcgtctcctca

SEQ ID NO: 6 - DNA, 360 bp, Artificial sequence
  Synthetic oligonucleotide
ctgcctgtgctgactcagccaccctcggtgtcagtgtccccaggacaaacggccaggatcacctgctctggagatacattggcaaagaaatatgcttattggtaccagcagaagtcaggccaggcccctgttctggtcatccaagacgacaccaagcgaccctccgggatccctcagcgattctctggctcaagctcagggacaatggccaccttgactataagtgcggcccaggtggaggatgaagctgactaccactgcttctcaacagatgatagtggaaatcctgagggcctcttcggcggaggaaccaaactgaccgtcctaagtcagcccaaggctgccccctcggtcactctg

SEQ ID NO: 7 - DNA, 363 bp, Artificial sequence
  Synthetic oligonucleotide
caggtgcagctggtggagtctgggggaggcgtggtccagcctgggaggtccctgagactctcctgtgcagcctctggattcaacttcattagttatggcatgcactgggtccgccaggctccaggcaaggggctggagtgggtggcacttatttcatatgatggaagtaataaatactatgcagactccgtgaagggccgattcaccatctccagagacaattccaagaacacgctgtatctgcaaatgaacagcctgagagctgaggacacggctgtatattactgtgcgaccgaggcttacgtggaaacagctatggtcccccagtactggggccagggaaccctggtcaccgtctcctca

SEQ ID NO: 8 - DNA, 321 bp, Artificial sequence
  Synthetic oligonucleotide
tcttatgagctgactcagccaccctcggtgtcagtgtccccaggacaaacggccaggatcacctgctctggagatgcattgccaaaagaatatgcttattggtaccagcagaagtcaggccaggcccctgtggtggtcatctatgaagacagcaaacgaccctccgggatccctgagcgattctctggctccagctcagggacaatggccaccttgactatcagtggggcccaggtggaggatgaagctgactaccactgttactcaacagacagcagtggtaatcccgtgttcggcggagggaccaagctgaccgtccta

SEQ ID NO: 9 - skipped

SEQ ID NO: 10 - protein, 129 aa, Artificial sequence
  Synthetic polypeptide
EVQLVQSGGGLVQPGGSLRLSCSASGFTFRTYAMSWVRQAPGKGLQWVSVISRSGDTTYHTDSVKGRFTISRDNSRNALYLQLDSLRAEDTALYYCAKTGNMAVGDRRTNYSYYYMDVWGKGTTVTVSS

SEQ ID NO: 11 - protein, 106 aa, Artificial sequence
  Synthetic polypeptide
DIVMTQSPSTLSASVGDRVTITCRASQSIKYWLAWYQQKPGKAPKLLIYKASNLESGVPSRFSGSGSGTEFTLTISSLRPDDFATYYCQQYNSYPLTFGGGTVEIK

SEQ ID NO: 12 - protein, 118 aa, Artificial sequence
  Synthetic polypeptide
EVQLVESGGGLVKPGGSLRLSCKASGFNFTNSWMSWVRQAPGKGLEWLGRIKSESDGGATRYAAPVTGRFSISRDDSRDMLFLQMNSLTTDDTAMYYCTTNKVTTNYWGQGTLVTVSS

SEQ ID NO: 13 - protein, 113 aa, Artificial sequence
  Synthetic polypeptide
DIVMTQSPVTLAVSLGERATINCKSSQSLLYSSDNENYLTWYQQKPGQPPKLLIYWASVRESGIPDRFIGSGSVTDFTLTINNVQAEDVAVYYCQQFRYTPLTFGQGTTLEIK

SEQ ID NO: 14 - protein, 113 aa, Artificial sequence
  Synthetic polypeptide
EVQLVESGAEVKRPGASVRVSCKASGYSFTLYYMHWVRQAPGQGLEWLGWINPKTGDVKYAQKFQGRVSLTRDTRMNTAYLDLTRLRSDDTARYYCLRAFDLWGRGTMIIVSS

SEQ ID NO: 15 - protein, 120 aa, Artificial sequence
  Synthetic polypeptide
LPVLTQPPSVSVSPGQTARITCSGDTLAKKYAYWYQQKSGQAPVLVIQDDTKRPSGIPQRFSGSSSGTMATLTISAAQVEDEADYHCFSTDDSGNPEGLFGGGTKLTVLSQPKAAPSVTL

SEQ ID NO: 16 - protein, 121 aa, Artificial sequence
  Synthetic polypeptide
QVQLVESGGGVVQPGRSLRLSCAASGFNFISYGMHWVRQAPGKGLEWVALISYDGSNKYYADSVKGRFTISRDNSKNTLYLQMNSLRAEDTAVYYCATEAYVETAMVPQYWGQGTLVTVSS

SEQ ID NO: 17 - protein, 107 aa, Artificial sequence
  Synthetic polypeptide
SYELTQPPSVSVSPGQTARITCSGDALPKEYAYWYQQKSGQAPVVVIYEDSKRPSGIPERFSGSSSGTMATLTISGAQVEDEADYHCYSTDSSGNPVFGGGTKLTVL

SEQ ID NO: 18 - protein, 8 aa, Artificial sequence
  Synthetic peptide
GFTFRTYA

SEQ ID NO: 19 - protein, 8 aa, Artificial sequence
  Synthetic peptide
ISRSGDTT

SEQ ID NO: 20 - protein, 13 aa, Artificial sequence
  Synthetic peptide
AKTGNMAVGDRRT

SEQ ID NO: 21 - protein, 8 aa, Artificial sequence
  Synthetic peptide
GFNFTNSW

SEQ ID NO: 22 - protein, 10 aa, Artificial sequence
  Synthetic peptide
IKSESDGGAT

SEQ ID NO: 23 - protein, 9 aa, Artificial sequence
  Synthetic peptide
TTNKVTTNY

SEQ ID NO: 24 - protein, 8 aa, Artificial sequence
  Synthetic peptide
GYSFTLYY

SEQ ID NO: 25 - protein, 8 aa, Artificial sequence
  Synthetic peptide
INPKTGDV

SEQ ID NO: 26 - protein, 6 aa, Artificial sequence
  Synthetic peptide
LRAFDL

SEQ ID NO: 27 - protein, 8 aa, Artificial sequence
  Synthetic peptide
GFNFISYG

SEQ ID NO: 28 - protein, 8 aa, Artificial sequence
  Synthetic peptide
ISYDGSNK

SEQ ID NO: 29 - protein, 14 aa, Artificial sequence
  Synthetic peptide
ATEAYVETAMVPQY

SEQ ID NO: 30 - protein, 6 aa, Artificial sequence
  Synthetic peptide
QSIKYW

SEQ ID NO: 31 - protein, 9 aa, Artificial sequence
  Synthetic peptide
QQYNSYPLT

SEQ ID NO: 32 - protein, 12 aa, Artificial sequence
  Synthetic peptide
QSLLYSSDNENY

SEQ ID NO: 33 - protein, 9 aa, Artificial sequence
  Synthetic peptide
QQFRYTPLT

SEQ ID NO: 34 - protein, 6 aa, Artificial sequence
  Synthetic peptide
TLAKKY

SEQ ID NO: 35 - protein, 12 aa, Artificial sequence
  Synthetic peptide
FSTDDSGNPEGL

SEQ ID NO: 36 - protein, 6 aa, Artificial sequence
  Synthetic peptide
ALPKEY

SEQ ID NO: 37 - protein, 10 aa, Artificial sequence
  Synthetic peptide
YSTDSSGNPV

SEQ ID NO: 38 - protein, 14 aa, Artificial sequence
  Synthetic peptide
PRIGGQRELKKITE

SEQ ID NO: 39 - protein, 23 aa, Artificial sequence
  Synthetic peptide
PRIGGQRELKKITEPGGSGGSGK

SEQ ID NO: 40 - protein, 14 aa, Artificial sequence
  Synthetic peptide
YGKDVKDLFDYAQE

SEQ ID NO: 41 - protein, 22 aa, Artificial sequence
  Synthetic peptide
YGKDVKDLFDYAQEGGSGGSGK

SEQ ID NO: 42 - DNA, 348 bp, Artificial Sequence
  Synthetic oligonucleotide
gaggtgcagctggtgcagtctggaggaggcttggtaaagcctggggggtcccttagactttcctgtgcagcctctggattcattttcagtaacgcctggatgaactgggtccgccaggctccagggaaggggctggagtgggttggccgtattaaaagagaaagtgatagtgggacaacagactacggtgcagccgtgaaaggcagattcaccatctcaagagatgattcaaaatacacgctgtatctgcaaatgaacagcctgaaaaccgacgacacagccgtttattactgtaccacagggtgggctgactactggggccagggaaccctggtcaccgtctcctca

SEQ ID NO: 43 - DNA, 312 bp, Artificial Sequence
  Synthetic oligonucleotide
ctgattcagccaccctcggtgtcagtgtccccaggacagacggccaggatcacctgctctggagatgcattgccaaacaaatatgcttattggtaccagcagaagccaggccaggccccttctgtggtgatgtttagagacaatgagagaccctcagggatccctgagcgattctctggctccagctcagggacaacagtcacgttgaccatcagtggagtccaggcagaagacgagtctgacttttattgtcaatccacagacagtaatggtgcttgggtgttcggcggagggaccaagctgaccgtccta

SEQ ID NO: 44 - DNA, 357 bp, Artificial Sequence
  Synthetic oligonucleotide
caggtgcagttggtgcagtctggggctgaggtgaagaagcctggggcctcagttcaagtttcctgcaggacatctggatacacctttattaattattttatgcactgggtgcgacaggcccctgggcaagggcttgagtggatgggaataatcaaccctaatggtggtaagacaagatacgcacagaagttccagggcagactcaccgtgaccagggacacgtccaccaacactgtctacgtggaactgagcaatctgagatatgaggacacgggcctctatttctgcgcgagagatccggagggggaagtgggctttgactactggggccagggaacccaggtcaccgtctcctca

SEQ ID NO: 45 - DNA, 324 bp, Artificial Sequence
  Synthetic oligonucleotide
tcccatgaactgacacagccaccctcggtgtcagtgtccccaggacagacggccaggatcacctgctctggagatgcactgtcaaagcaatatgcttattggtatcagcagaagccaggccaggcccctgtggtggtgatatataaagacaatgagaggccctcagggatccctgagcgattctctggctccagttcaggcacaacagtcacattgaccatcactggagtccaggcagaagacgaggctgactattattgtcaatcaacagacaccagtcgtgcttattatgtcttcggaactgggaccaaggtcaccgtctta

SEQ ID NO: 46 - protein, 116 aa, Artificial Sequence
  Synthetic polypeptide
EVQLVQSGGGLVKPGGSLRLSCAASGFIFSNAWMNWVRQAPGKGLEWVGRIKRESDSGTTDYGAAVKGRFTISRDDSKYTLYLQMNSLKTDDTAVYYCTTGWADYWGQGTLVTVSS

SEQ ID NO: 47 - protein, 104 aa, Artificial Sequence
  Synthetic polypeptide
LIQPPSVSVSPGQTARITCSGDALPNKYAYWYQQKPGQAPSVVMFRDNERPSGIPERFSGSSSGTTVTLTISGVQAEDESDFYCQSTDSNGAWVFGGGTKLTVL

SEQ ID NO: 48 - protein, 119 aa, Artificial Sequence
  Synthetic polypeptide
QVQLVQSGAEVKKPGASVQVSCRTSGYTFINYFMHWVRQAPGQGLEWMGIINPNGGKTRYAQKFQGRLTVTRDTSTNTVYVELSNLRYEDTGLYFCARDPEGEVGFDYWGQGTQVTVSS

SEQ ID NO: 49 - protein, 108 aa, Artificial Sequence
  Synthetic polypeptide
SHELTQPPSVSVSPGQTARITCSGDALSKQYAYWYQQKPGQAPVVVIYKDNERPSGIPERFSGSSSGTTVTLTITGVQAEDEADYYCQSTDTSRAYYVFGTGTKVTVL

SEQ ID NO: 50 - protein, 8 aa, Artificial Sequence
  Synthetic polypeptide
GFIFSNAW

SEQ ID NO: 51 - protein, 10 aa, Artificial Sequence
  Synthetic polypeptide
IKRESDSGTT

SEQ ID NO: 52 - protein, 7 aa, Artificial Sequence
  Synthetic polypeptide
TTGWADY

SEQ ID NO: 53 - protein, 5 aa, Artificial Sequence
  Synthetic polypeptide
NYFMH

SEQ ID NO: 54 - protein, 17 aa, Artificial Sequence
  Synthetic polypeptide
IINPNGGKTRYAQKFQG

SEQ ID NO: 55 - protein, 10 aa, Artificial Sequence
  Synthetic polypeptide
DPEGEVGFDY

SEQ ID NO: 56 - protein, 6 aa, Artificial Sequence
  Synthetic polypeptide
ALPNKY

SEQ ID NO: 57 - protein, 10 aa, Artificial Sequence
  Synthetic polypeptide
QSTDSNGAWV

SEQ ID NO: 58 - protein, 11 aa, Artificial Sequence
  Synthetic polypeptide
SGDALSKQYAY

SEQ ID NO: 59 - protein, 7 aa, Artificial Sequence
  Synthetic polypeptide
KDNERPS

SEQ ID NO: 60 - protein, 11 aa, Artificial Sequence
  Synthetic polypeptide
QSTDTSRAYYV

SEQ ID NO: 61 - protein, 135 aa, Artificial Sequence
  Synthetic polypeptide
MGWSYIILFLLATATRVHSQVQLQQPGAEVVRPGASVKVSCKASGYTVSSYWMSWVKQRPEQGLEWIGRIDPYDSETHYNQKFKDKAILTVDKSSSTAYMQLSSLTSEDSAVYYCARTAASFDYWGQGTTLTVSS

SEQ ID NO: 62 - protein, 131 aa, Artificial Sequence
  Synthetic polypeptide
MKLPVRLLVLMFWIPASSSDVVMTQTPLSLPVSLGDQASISCRSSQSLVHSNGNSYLHWYLQKPGQSPKLLIYKVSNRFSGVPDRFSGSGSGTDFTLNISRVEAEDLGVYFCSQSTHVPFTFGSGTKLEIK

SEQ ID NO: 63 - DNA, 405 bp, Artificial Sequence
  Synthetic polynucleotide
atgggatggagctatatcatcctcttcttgttagcaacagctacacgtgtccactcccaggtccaactgcagcagcctggggctgaggtggtgaggcctggggcttcagtgaaggtgtcctgcaaggcttctggctacacggtcagcagctactggatgagctgggttaagcagaggccggagcaaggccttgagtggattggaaggattgatccttacgatagtgaaactcactacaatcaaaagttcaaggacaaggccatattgactgtagacaaatcctccagcacagcctacatgcaactcagcagcctgacatctgaggactctgcggtctattactgtgcaaggacggccgcttcgtttgactattggggccaaggcaccactctcacagtctcctca

SEQ ID NO: 64 - DNA, 393 bp, Artificial Sequence
  Synthetic polynucleotide
atgaagttgcctgttaggctgttggtgctgatgttctggattcctgcttccagcagtgatgttgtgatgacccaaactccactctccctgcctgtcagtcttggagatcaagcctccatctcttgcagatctagtcagagccttgtacacagtaatggaaactcctatttacattggtacctgcagaagccaggccagtctccaaagctcctgatctacaaagtttccaaccgattttctggggtcccagacaggttcagtggcagtggatcagggacagatttcacactcaatatcagcagagtggaggctgaggatctgggagtttatttctgctctcaaagtacacatgttccattcacgttcggctcggggacaaagttggaaataaaa

SEQ ID NO: 65 - protein, 134 aa, Artificial Sequence
  Synthetic polypeptide
MGWSYIILFLLATATRVHSQVQLQQPGAEVVRPGASVKVSCKASGYTVSSYWMSWVKQRPEQGLEWIGRIDPYDSETHYNQKFKDKAILTVKSSSTAYMQLSSLTSEDSAVYYCARTAASFDYWGQGTTLTVSS

SEQ ID NO: 66 - protein, 138 aa, Artificial Sequence
  Synthetic polypeptide
MERHWIFLFLLSVTAGVHSQVQLQQSAAELARPGASVKMSCKASGYTFSSYTMHWVKRPGQGLEWIGYINPSSGYTDYNQKFKDKTTLTADKSSSTAYMQLSSLTSEDSAVYYCRLYDNYDYYAMDYWGQGTSVTVSS

SEQ ID NO: 67 - protein, 130 aa, Artificial Sequence
  Synthetic polypeptide
MDSQAQVLILLLLWVSGTCGDIVMSQSPSSLAVSAGEKVTMSCKSSQSLLNSRIRKNLAWYQQKPGQSPKLLIYWASTRESGVPDRFTGSGSGTDFTLTISSVQADDLAVYYCKQYNLLTFGAGTKLELK

SEQ ID NO: 68 - DNA, 420 bp, Artificial Sequence
  Synthetic polynucleotide
atggaaaggcactggatctttctcttcctgttgtcagtaactgcaggtgtccactcccaggtccagctgcagcagtctgcagctgaactggcaagacctggggcctcagtgaagatgtcctgcaaggcttctggctacacctttagtagctacacgatgcactgggtaaaacagaggcctggacagggtctggaatggattggatacattaatcctagcagtggatatactgattacaatcagaagttcaaggacaagaccacattgactgcagacaaatcctccagcacagcctacatgcaactgagcagcctgacatctgaggactctgcggtctattactgtgcaagactatatgataactacgattactatgctatggactactggggtcaaggaacctcagtcaccgtctcctca

SEQ ID NO: 69 - protein, 140 aa, Artificial Sequence
  Synthetic polypeptide
MERHWIFLFLLSVTAGVHSQVQLQQSAAELARPGASVKMSCKASGYTFSSYTMHWVKQRPGQGLEWIGYINPSSGYTDYNQKFKDKTTLTADKSSSTAYMQLSSLTSEDSAVYYCARLYDNYDYYAMDYWGQGTSVTVSS

SEQ ID NO: 70 - DNA, 396 bp, Artificial Sequence
  Synthetic polynucleotide
atggattcacaggcccaggttcttatattgctgctgctatgggtatctggtacctgtggggacattgtgatgtcacagtctccatcctccctggctgtgtcagcaggagagaaggtcactatgagctgcaaatccagtcagagtctgctcaatagtagaatccgaaagaactacttggcttggtaccagcagaaaccagggcagtctcctaaactgctgatctactgggcatccactagggaatctggggtccctgatcgcttcacaggcagtggatctgggacagatttcactctcaccatcagcagtgtgcaggctgatgacctggcagtttattactgcaagcaatcttataatctgctcacgttcggtgctgggaccaagctggagctgaaa

SEQ ID NO: 71 - protein, 132 aa, Artificial Sequence
  Synthetic polypeptide
MDSQAQVLILLLLWVSGTCGDIVMSQSPSSLAVSAGEKVTMSCKSSQSLLNSRIRKNYLAWYQQKPGQSPKLLIYWASTRESGVPDRFTGSGSGTDFTLTISSVQADDLAVYYCKQSYNLLTFGAGTKLELK

SEQ ID NO: 72 - protein, 139 aa, Artificial Sequence
  Synthetic polypeptide
MERHWIFLFLLSVTAGVHSQVQLQQSAAELARPGASVKMSCKASGYTFSSYTMHWVKQRPGQGLEWIGYINPSSGYTDYNQKFKDKTTLTAKSSSTAYMQLSSLTSEDSAVYYCARLYDNYDYYAMDYWGQGTSVTVSS

(19) United States
(12) Patent Application Publication
(10) Pub. No.: US 2022/0280618 A1
(43) Pub. Date: Sep. 8, 2022

(54) ANTIBODIES TO CANDIDA AND USES THEREOF

(71) Applicants: The Administrators of the Tulane Educational Fund, New Orleans, LA (US); The Board of Supervisors of Louisiana State University and Agricultural and Mechanical College, Baton Rouge, LA (US); Autoimmune Technologies, LLC, New Orleans, LA (US)

(72) Inventors: Russell B. WILSON, New Orleans, LA (US); Hong XIN, New Orleans, LA (US); James E. ROBINSON, New Orleans, LA (US)

(73) Assignees: The Administrators of the Tulane Educational Fund, New Orleans, LA (US); The Board of Supervisors of Louisiana State University and Agricultural and Mechanical College, Baton Rouge, LA (US); Autoimmune Technologies, LLC, New Orleans, LA (US)

(21) Appl. No.: 17/631,433

(22) PCT Filed: Jul. 28, 2020
(86) PCT No.: PCT/US2020/043908

§ 371 Date: Jan. 28, 2022

Related U.S. Application Data
(60) Provisional application No. 62/879,912, filed on Jul. 29, 2019.
(60) Provisional application No. 62/879,894, filed on Jul. 29, 2019.

(51) Int. Cl. A61K 39/00; C07K 16/14; G01N 33/569

(52) U.S. Cl. CPC A61K 39/0002; C07K 16/14; G01N 33/56961; A61K 2039/505; C07K 2317/21; C07K 2317/34; C07K 2317/76; C07K 2317/92; G01N 2333/40; G01N 2469/10

(57) ABSTRACT

The present invention is directed to antibodies binding to and neutralizing Candida and methods for use thereof.

### PRIORITY CLAIM

This application claims benefit of priority to U.S. Provisional Application Ser. Nos. 62/879,894 and 62/879,912, both filed on Jul. 29, 2019, the entire contents of both applications being hereby incorporated by reference.

### BACKGROUND

### 1. Field of the Disclosure

The present invention relates generally to the fields of medicine, infectious disease, and immunology. More particular, the disclosure relates to human antibodies binding to Candida spp and their use in treating subjects with disseminated candidiasis.

### 2. Background

The most common causes of invasive fungal infections are members of the genus Candida (Kim and Sudbery, 2011). Disseminated candidiasis ranks third of all nosocomial bloodstream infections and, despite antifungal therapy, at least 40% of affected individuals will die of this disease, and it is the cause of more case fatalities than any other systemic mycosis. It is estimated that 60,000-70,000 cases of disseminated candidiasis occur per year in the US alone, and associated health care costs are $2-4 billion/year. There are numerous species of Candida that are human pathogens with the most medically relevant being: C. albicans, the most common species identified (˜60%); C. glabrata (˜15-20%); C. parapsilosis (˜10-20%), mostly found in hospitalized patients with vascular catheters; C. tropicalis (˜6-12%), often found in patients with cancer (leukemia), and those who have undergone bone marrow transplantation; C. guilliermondi (<5%); C. lustianiae (<5%); and C. dubliniensis, found primarily in patients who are positive for HIV.

Concern is rising about the high incidence of infections caused by non-albicans species and the emergence of antifungal resistance. Among the non-albicans species, C. tropicalis and C. parapsilosis are both generally susceptible to azoles; however, C. tropicalis is less susceptible to Fluconazole™ than is C. albicans. C. glabrata is intrinsically more resistant to antifungal agents, particularly to Fluconazole™. C. krusei is intrinsically resistant to Fluconazole™, and infections caused by this species are strongly associated with prior Fluconazole™ prophylaxis and neutropenia. (Turner and Butler, 2014). In addition, the incidence of reported infections of C. auris, an emerging multidrug resistant strain recently identified, appears to be increasing at a rapid rate (Chowdhary et al., 2013). Invasive mycosis following solid organ transplantation, in particular, is also a significant problem with the incidence of up to 40%, depending on the transplant type, and rates of morbidity and mortality between 25% and 95% depending on the organ and type of fungus (Low and Rotstein, 2011).

Given the high mortality rate and significant burden on the healthcare system associated with disseminated candidiasis, new approaches are needed to supplement or replace current antifungal therapy. One approach is the use of antibodies to treat or prevent candida infection. This possibility is supported by several lines of evidence that indicate that antibodies to C. albicans contribute to host defense against disseminated candidiasis: B cell depleted mice show increased susceptibility to candida, and immunoglobulin (IVIG) therapy is associated with a lower incidence of candidiasis in liver transplant (Casadevall et al., 2002).

A human recombinant single chain antibody fragment (SCFV), called Efungumab (Mycograb™) was being developed as an immunotherapeutic for disseminated candidiasis (Karwa and Wargo, 2009). This SCFV bound to the heat shock protein HSP70 from candida and increased the effectiveness of Amphotericin B. This drug was twice denied regulatory approval due to manufacturing issues and a modified version, where a free cystine residue was removed, was tested. Enhancement of Amphotericin B activity was detected but found to be non-specific (Richie et al., 2012). Further development of Efungumab has been dropped. More recently, several human monoclonal antibodies to Hyr1, a candida cell wall protein, and to other unidentified cell wall proteins have been isolated and described (Rudkin et al., 2018). These antibodies protect after passive transfer in mouse models of disseminated candidiasis, however, they function by opsonization and enhance the phagocytosis of C. albicans. This mode of action may be a drawback in using these antibodies as therapeutics in immunosuppressed or immunocompromised patients where macrophage or neutrophil function may be compromised and, in addition, C. albicans has mechanisms for reducing complement-mediated adhesion and uptake of C. albicans through the function of Pra1 (Luo et al., 2010).

Additional evidence for a role of antibodies stems from the work on the development of glycopeptide-based vaccines to protect from Candida infections. For example, six putative T-cell peptides found in C. albicans cell wall proteins were conjugated to the protective β-1,2-mannotriose [β-(Man)₃] glycan epitope to create glycopeptide conjugates (Xin et al., 2008). The six proteins from which the peptides, denoted in parentheses, were derived are cell wall-associated proteins including: fructose-bisphosphate aldolase (Fba) (YGKDVKDLDYAQE; SEQ ID NO: 40); methyltetrahydropteroyltriglutamate homocysteine methyltransferase (MET6) (PRIGGQRELKKITE; SEQ ID NO: 38) in addition to four other proteins (Xin et al., 2008). The intent of this work was to use the peptides as T-cell epitopes, promoting protective antibody responses against the glycan part of the glycopeptide conjugates. Thus, the immunization protocols were designed to favor antibody, rather than cell-mediated immune (CMI) responses and antibodies were generated against both the glycan and peptide parts of the various conjugates. Three of the glycoconjugates including the β-(Man)₃-Fba and β-(Man)₃-Meth conjugates induced protection from hematogenous challenge with the fungus as evidenced by mouse survival and low kidney fungal burden. In addition, mouse monoclonal antibodies generated to the Fba and Met6 peptides, alone, protected mice as well following passive transfer (Xin et al., 2008).

Many candida proteins have been identified as pathogenic factors (Mayer, Wilson, and Hube, 2014), including the moonlighting proteins fructose-bisphosphate aldolase (Fba) and 5-methyltetrahydropteroyltriglutamate homocysteine methyltransferase (Met6), (Gancedo et al., 2016; Medrano-Diaz, et al., 2018). These metabolic enzymes are normally located intracellularly, but by unknown mechanisms are also secreted and bind to the fungal cell wall where they serve as virulence factors. Moonlighting proteins function as virulence factors through a variety of mechanisms including binding of plasminogen, fibronectin, extracellular matrix proteins, or inhibitors of complement fixation, or serve as adhesion molecules to bind to host cells to recruit inflammatory responses. Thus, these virulence factors allow pathogenic Candida sp. the ability to invade and escape host defense mechanisms (Henderson and Martin, 2011).

U.S. Pat. Nos. 6,309,642, 6,391,587, and 6,403,090 and U.S. Patent Application Publication U.S. 2003/0072775 disclose vaccines based on peptides that mimic phosphormanna epitopes or polynucleotides encoding the peptide mimotopes, and discloses mouse monoclonal antibodies, including MAb B6.1, for passive immunization against infections of Candida albicans.

### SUMMARY

Thus, in accordance with the present invention, there is provided a method of detecting a Candida infection in a subject. In embodiments, the method comprises (a) contacting a sample from said subject with an antibody or antibody fragment having clone-paired heavy and light chain CDR sequences from Tables 3 and 4, respectively; (b) detecting Candida in said sample by binding of said antibody or antibody fragment to a Candida antigen in said sample, or a combination thereof. The sample can be a body fluid, such as blood, sputum, tears, saliva, mucous or serum, semen, cervical or vaginal secretions, amniotic fluid, placental tissues, urine, exudate, transudate, tissue scrapings or feces. Detection can comprise ELISA, RIA, lateral flow assay or Western blot. The method can further comprise performing steps (a) and (b) a second time and determining a change in Candida antigen levels as compared to the first assay. The Candida may be any pathogenic Candida species, including but not limited to C. albicans, C. glabrata, C. tropicalis or C. auris.

In embodiments, the antibody or antibody fragment can be encoded by any of the clone-paired variable sequences as set forth in Table 1. The antibody or antibody fragment can be encoded by variable sequences with at least 70% identity to the sequences set forth in Table 1. In certain embodiments, the antibody or antibody fragment is encoded by light and heavy chain variable sequences having about 70%, about 80%, or about 90% identity to clone-paired variable sequences as set forth in Table 1. The antibody or antibody fragments can be encoded by light and heavy chain variable sequences having about 95% identity to clone-paired sequences as set forth in Table 1. In embodiments, the antibody or antibody fragments comprise light and heavy chain variable sequences according to clone-paired sequences from Table 2. The antibody or antibody fragment comprise variable sequences with at least 70% identity to the sequences set forth in Table 2. In certain embodiments, the antibody or antibody fragment can comprise light and heavy chain variable sequences having about 70%, about 80% or about 90% identity to clone-paired sequences from Table 2. The antibody or antibody fragments can comprise light and heavy chain variable sequences having about 95% identity to clone-paired sequences from Table 2 or can comprise light and heavy chain variable sequences according to clone-paired sequences from Table 2. The antibody fragment can be a recombinant scFv (single chain fragment variable) antibody, Fab fragment, F(ab′)₂ fragment, or Fv fragment.

In another embodiment, there is provided a method of treating a subject infected with Candida or reducing the likelihood of infection of a subject at risk of contracting Candida comprising delivering to said subject an antibody or antibody fragment having clone-paired heavy and light chain CDR sequences from Tables 3 and 4, respectively. The antibody or antibody fragment can have clone-paired CDRs with at least 70% identity to sequences set forth in Tables 3 and 4. In certain embodiments, the antibody or antibody fragment has clone-paired CDRs with about 70%, about 80%, or about 90% identical to the sequences from Tables 3 and 4. The antibody or antibody fragment can be encoded by clone-paired variable sequences as set forth in Table 1. The antibody or antibody fragment can be encoded by variable sequences with at least 70% identity to the sequences set forth in Table 1, In certain embodiments, the antibody or antibody fragment is encoded by light and heavy chain variable sequences having about 70%, about 80%, or about 90% identity to clone-paired variable sequences as set forth in Table 1. The antibody or antibody fragments can be encoded by light and heavy chain variable sequences having about 95% identity to clone-paired sequences as set forth in Table 1. In embodiments, the antibody or antibody fragments comprise light and heavy chain variable sequences according to clone-paired sequences from Table 2. The antibody or antibody fragment can comprise variable sequences with at least 70% identity to the sequences set forth in Table 2. In certain embodiments, the antibody or antibody fragment can comprise light and heavy chain variable sequences having about 70%, about 80% or about 90% identity to clone-paired sequences from Table 2. The antibody or antibody fragments can comprise light and heavy chain variable sequences having about 95% identity to clone-paired sequences from Table 2 or can comprise light and heavy chain variable sequences according to clone-paired sequences from Table 2. The Candida may be any pathogenic Candida species, including but not limited to C. albicans, C. glabrata, C. tropicalis or C. auris.

The antibody fragment can be a recombinant scFv (single chain fragment variable) antibody. Fab fragment, F(ab′)₂ fragment, or Fv fragment. The antibody can be a chimeric antibody, or a bispecific antibody. The antibody can be an IgG, or a recombinant IgG antibody or antibody fragment comprising an Fc portion mutated to alter (eliminate or enhance) FcR interactions, to increase half-life and/or increase therapeutic efficacy, such as a LALA, N297, GASD/ALIE, YTE or LS mutation or glycan modified to alter (eliminate or enhance) FcR interactions such as enzymatic or chemical addition or removal of glycans or expression in a cell line engineered with a defined glycosylating pattern. The antibody or antibody fragment can further comprise a cell penetrating peptide. The antibody or antibody fragment can be an intrabody.

The antibody or antibody fragment can be administered prior to infection or after infection. The subject can be a pregnant female, a sexually active female, or a female undergoing fertility treatments. Delivering can comprise antibody or antibody fragment administration, or genetic delivery with an RNA or DNA sequence or vector encoding the antibody or antibody fragment.

In yet another embodiment, there is provided a monoclonal antibody, wherein the antibody or antibody fragment is characterized by clone-paired heavy and light chain CDR sequences from Tables 3 and 4, respectively. The antibody or antibody fragment can have clone-paired CDRs with at least 70% identity to sequences set forth in Tables 3 and 4. In certain embodiments, the antibody or antibody fragment has clone-paired CDRs with about 70%, about 80%, or about 90% identical to the sequences from Tables 3 and 4. The antibody or antibody fragment can be encoded by clone-paired variable sequences as set forth in Table 1. The antibody or antibody fragment can be encoded by variable sequences with at least 70% identity to the sequences set forth in Table 1. In certain embodiments, the antibody or antibody fragment is encoded by light and heavy chain variable sequences having about 70%, about 80%, or about 90% identity to clone-paired variable sequences as set forth in Table 1. The antibody or antibody fragments can be encoded by light and heavy chain variable sequences having about 95% identity to clone-paired sequences as set forth in Table 1. In embodiments, the antibody or antibody fragments comprise light and heavy chain variable sequences according to clone-paired sequences from Table 2. The antibody or antibody fragment can comprise variable sequences with at least 70% identity to the sequences set forth in Table 2. In certain embodiments, the antibody or antibody fragment can comprise light and heavy chain variable sequences having about 70%, about 80% or about 90% identity to clone-paired sequences from Table 2. The antibody or antibody fragments can comprise light and heavy chain variable sequences having about 95% identity to clone-paired sequences from Table 2 or can comprise light and heavy chain variable sequences according to clone-paired sequences from Table 2. The Candida may be any pathogenic Candida species, including but not limited to C. albicans, C. glabrata, C. tropicalis or C. auris.

The antibody fragment can be a recombinant scFv (single chain fragment variable) antibody, Fab fragment, F(ab′)₂ fragment, or Fv fragment. The antibody can be a chimeric antibody, or a bispecific antibody. The antibody can be an IgG, or a recombinant IgG antibody or antibody fragment comprising an Fc portion mutated to alter (eliminate or enhance) FcR interactions, to increase half-life and/or increase therapeutic efficacy, such as a LALA, N297, GASD/ALIE, YTE or LS mutation or glycan modified to alter (eliminate or enhance) FcR interactions such as enzymatic or chemical addition or removal of glycans or expression in a cell line engineered with a defined glycosylating pattern. The antibody or antibody fragment can further comprise a cell penetrating peptide. The antibody or antibody fragment can be an intrabody.

In still yet another embodiment, there is provided a hybridoma or engineered cell encoding an antibody or antibody fragment wherein the antibody or antibody fragment is characterized by clone-paired heavy and light chain CDR sequences from Tables 3 and 4, respectively. The antibody or antibody fragment can have clone-paired CDRs with at least 70% identity to sequences set forth in Tables 3 and 4. In certain embodiments, the antibody or antibody fragment has clone-paired CDRs with about 70%, about 80%, or about 90% identical to the sequences from Tables 3 and 4. The antibody or antibody fragment can be encoded by clone-paired variable sequences as set forth in Table 1. The antibody or antibody fragment can be encoded by variable sequences with at least 70% identity to the sequences set forth in Table 1. In certain embodiments, the antibody or antibody fragment is encoded by light and heavy chain variable sequences having about 70%, about 80%, or about 90% identity to clone-paired variable sequences as set forth in Table 1. The antibody or antibody fragments can be encoded by light and heavy chain variable sequences having about 95% identity to clone-paired sequences as set forth in Table 1. In embodiments, the antibody or antibody fragments can comprise light and heavy chain variable sequences according to clone-paired sequences from Table 2. The antibody or antibody fragment can comprises variable sequences with at least 70% identity to the sequences set forth in Table 2. In certain embodiments, the antibody or antibody fragment can comprise light and heavy chain variable sequences having about 70%, about 80% or about 90% identity to clone-paired sequences from Table 2. The antibody or antibody fragments can, may comprise light and heavy chain variable sequences having about 95% identity to clone-paired sequences from Table 2, or can comprise light and heavy chain variable sequences according to clone-paired sequences from Table 2.

The antibody fragment can be a recombinant scFv (single chain fragment variable) antibody, Fab fragment, F(ab′)₂ fragment, or Fv fragment. The antibody can be a chimeric antibody, or a bispecific antibody. The antibody can be an IgG, or a recombinant IgG antibody or antibody fragment comprising an Fc portion mutated to alter (eliminate or enhance) FcR interactions, to increase half-life and/or increase therapeutic efficacy, such as a LALA, N297, GASD/ALIE, YTE or LS mutation or glycan modified to alter (eliminate or enhance) FcR interactions such as enzymatic or chemical addition or removal of glycans or expression in a cell line engineered with a defined glycosylating pattern. The antibody or antibody fragment can further comprise a cell penetrating peptide. The antibody or antibody fragment can be intrabody.

In a further embodiment, there is provided a vaccine formulation comprising one or more antibodies or antibody fragments characterized by clone-paired heavy and light chain CDR sequences from Tables 3 and 4, respectively. The antibody or antibody fragment can have clone-paired CDRs with at least 70% identity to sequences set forth in Tables 3 and 4. In certain embodiments, the antibody or antibody fragment has clone-paired CDRs with about 70%, about 80%, or about 90% identical to the sequences from Tables 3 and 4. The antibody or antibody fragment can be encoded by clone-paired variable sequences as set forth in Table 1. The antibody or antibody fragment can be encoded by variable sequences with at least 70% identity to the sequences set forth in Table 1. In certain embodiments, the antibody or antibody fragment is encoded by light and heavy chain variable sequences having about 70%, about 80%, or about 90% identity to clone-paired variable sequences as set forth in Table 1. The antibody or antibody fragments can be encoded by light and heavy chain variable sequences having about 95% identity to clone-paired sequences as set forth in Table 1. In embodiments, the antibody or antibody fragments can comprise light and heavy chain variable sequences according to clone-paired sequences from Table 2. The antibody or antibody fragment can comprise variable sequences with at least 70% identity to the sequences set forth in Table 2. In certain embodiments, the antibody or antibody fragment can comprise light and heavy chain variable sequences having about 70%, about 80% or about 90% identity to clone-paired sequences from Table 2. The antibody or antibody fragments can, may comprise light and heavy chain variable sequences having about 95% identity to clone-paired sequences from Table 2, or can comprise light and heavy chain variable sequences according to clone-paired sequences from Table 2.

The antibody fragment can be a recombinant scFv (single chain fragment variable) antibody, Fab fragment, F(ab′)₂ fragment, or Fv fragment. The antibody can be a chimeric antibody, or a bispecific antibody. The antibody can be an IgG, or a recombinant IgG antibody or antibody fragment comprising an Fc portion mutated to alter (eliminate or enhance) FcR interactions, to increase half-life and/or increase therapeutic efficacy, such as a LALA, N297, GASD/ALIE, YTE or LS mutation or glycan modified to alter (eliminate or enhance) FcR interactions such as enzymatic or chemical addition or removal of glycans or expression in a cell line engineered with a defined glycosylating pattern. The antibody or antibody fragment can further comprise a cell penetrating peptide. The antibody or antibody fragment can be an intrabody.

In still another embodiment, there is provided a vaccine formulation comprising one or more expression vectors encoding a first antibody or antibody fragment as described herein. The expression vector(s) can be Sindbis virus or VEE vector(s). The vaccine can be formulated for delivery by needle injection, jet injection, or electroporation. The vaccine can further comprise one or more expression vectors encoding for a second antibody or antibody fragment, such as a distinct antibody or antibody fragment as described herein.

And additional embodiment comprises a method of protecting the health of a placenta and/or fetus of a pregnant a subject infected with or at risk of infection with Candida comprising delivering to said subject an antibody or antibody fragment having clone-paired heavy and light chain CDR sequences from Tables 3 and 4, respectively. The antibody or antibody fragment can have clone-paired CDRs with at least 70% identity to sequences set forth in Tables 3 and 4. In certain embodiments, the antibody or antibody fragment has clone-paired CDRs with about 70%, about 80%, or about 90% identical to the sequences from Tables 3 and 4. The antibody or antibody fragment can be encoded by clone-paired variable sequences as set forth in Table 1. The antibody or antibody fragment can be encoded by variable sequences with at least 70% identity to the sequences set forth in Table 1. In certain embodiments, the antibody or antibody fragment is encoded by light and heavy chain variable sequences having about 70%, about 80%, or about 90% identity to clone-paired variable sequences as set forth in Table 1.The antibody or antibody fragments can be encoded by light and heavy chain variable sequences having about 95% identity to clone-paired sequences as set forth in Table 1. In embodiments, the antibody or antibody fragments can comprise light and heavy chain variable sequences according to clone-paired sequences from Table 2. The antibody or antibody fragment can comprise variable sequences with at least 70% identity to the sequences set forth in Table 2. In certain embodiments, the antibody or antibody fragment can comprise light and heavy chain variable sequences having about 70%, about 80% or about 90% identity to clone-paired sequences from Table 2. The antibody or antibody fragments can, may comprise light and heavy chain variable sequences having about 95% identity to clone-paired sequences from Table 2, or can comprise light and heavy chain variable sequences according to clone-paired sequences from Table 2. The Candida may be any pathogenic Candida species, including but not limited to C. albicans, C. glabrata, C. tropicalis or C. auris.

The antibody fragment can be a recombinant scFv (single chain fragment variable) antibody, Fab fragment, F(ab′)₂ fragment, or Fv fragment. The antibody can be a chimeric antibody, or a bispecific antibody. The antibody can be an IgG, or a recombinant IgG antibody or antibody fragment comprising an Fc portion mutated to alter (eliminate or enhance) FcR interactions, to increase half-life and/or increase therapeutic efficacy, such as a LALA, N297, GASD/ALIE, YTE or LS mutation or glycan modified to alter (eliminate or enhance) FcR interactions such as enzymatic or chemical addition or removal of glycans or expression in a cell line engineered with a defined glycosylating pattern. The antibody or antibody fragment can further comprise a cell penetrating peptide. The antibody or antibody fragment can be an intrabody.

The antibody or antibody fragment can be administered prior to infection or after infection. The subject can be a pregnant female, a sexually active female, or a female undergoing fertility treatments. Delivering can comprise antibody or antibody fragment administration, or genetic delivery with an RNA or DNA sequence or vector encoding the antibody or antibody fragment. The antibody or antibody fragment can increase the size of the placenta as compared to an untreated control or can reduce fungal load and/or pathology of the fetus as compared to an untreated control.

Another embodiment comprises a method of determining the antigenic integrity, correct conformation and/or correct sequence of a Candida antigen comprising (a) contacting a sample comprising said antigen with a first antibody or antibody fragment having clone-paired heavy and light chain CDR sequences from Tables 3 and 4, respectively; and (b) determining antigenic integrity, correct conformation and/or correct sequence of said antigen by detectable binding of said first antibody or antibody fragment to said antigen. The sample can comprise recombinantly produced antigen, or a vaccine formulation or vaccine production batch. Detection can comprise ELISA, RIA, western blot, a biosensor using surface plasmon resonance or biolayer interferometry, or flow cytometric staining. The method can further comprise performing steps (a) and (b) a second time to determine the antigenic stability of the antigen over time. The Candida may be any pathogenic Candida species, including but not limited to C. albicans, C, glabrata, C. tropicalis or C. auris.

The first antibody or antibody fragment can be encoded by clone-paired variable sequences as set forth in Table 1. The antibody or antibody fragment can be encoded by variable sequences with at least 70% identity to the sequences set forth in Table 1. In certain embodiments, the antibody or antibody fragment is encoded by light and heavy chain variable sequences having about 70%, about 80%, or about 90% identity to clone-paired variable sequences as set forth in Table 1. The antibody or antibody fragments can be encoded by light and heavy chain variable sequences having about 95% identity to clone-paired sequences as set forth in Table 1. In embodiments, the antibody or antibody fragments can comprise light and heavy chain variable sequences according to clone-paired sequences from Table 2. The antibody or antibody fragment can comprise variable sequences with at least 70% identity to the sequences set forth in Table 2. In certain embodiments, the antibody or antibody fragment can comprise light and heavy chain variable sequences having about 70%, about 80% or about 90% identity to clone-paired sequences from Table 2. The antibody or antibody fragments can, may comprise light and heavy chain variable sequences having about 95% identity to clone-paired sequences from Table 2, or can comprise light and heavy chain variable sequences according to clone-paired sequences from Table 2. The first antibody fragment can be a recombinant scFv (single chain fragment variable) antibody, Fab fragment, F(ab′)₂ fragment, or Fv fragment.

The method can further comprise (c) contacting a sample comprising said antigen with a second antibody or antibody fragment having clone-paired heavy and light chain CDR sequences from Tables 3 and 4, respectively; and (d) determining antigenic integrity of said antigen by detectable binding of said second antibody or antibody fragment to said antigen. Detection can comprise ELISA, RIA, western blot, a biosensor using surface plasmon resonance or biolayer interferometry, or flow cytometric staining. The method can further comprise performing steps (c) and (d) a second time to determine the antigenic stability of the antigen over time.

The second antibody or antibody fragment can be encoded by clone-paired variable sequences as set forth in Table 1. The antibody or antibody fragment can be encoded by variable sequences with at least 70% identity to the sequences set forth in Table 1. In certain embodiments, the antibody or antibody fragment is encoded by light and heavy chain variable sequences having about 70%, about 80%, or about 90% identity to clone-paired variable sequences as set forth in Table 1. The antibody or antibody fragments can be encoded by light and heavy chain variable sequences having about 95% identity to clone-paired sequences as set forth in Table 1. In embodiments, the antibody or antibody fragments can comprise light and heavy chain variable sequences according to clone-paired sequences from Table 2. The antibody or antibody fragment can comprise variable sequences with at least 70% identity to the sequences set forth in Table 2. In certain embodiments, the antibody or antibody fragment can comprise light and heavy chain variable sequences having about 70%, about 80% or about 90% identity to clone-paired sequences from Table 2. The antibody or antibody fragments can, may comprise light and heavy chain variable sequences having about 95% identity to clone-paired sequences from Table 2, or can comprise light and heavy chain variable sequences according to clone-paired sequences from Table 2. The second antibody fragment can be a recombinant scFv (single chain fragment variable) antibody, Fab fragment, (ab′)₂ fragment, or Fv fragment.

Additionally, there is provided monoclonal antibody or fragment thereof, wherein the antibody or antibody fragment comprises clone-paired heavy and light chain CDR sequences, wherein the heavy chain CDR sequences are selected from Table 3, and wherein the light chain CDR sequences are selected from Table 4, and wherein the antibody or fragment thereof specifically binds its cognate antigen via its VL and/or VH paratope comprising at least 5 amino acids from the red or orange ribbons depicted in the ribbon diagrams selected from group consisting of FIG. 10, FIG. 11, FIG. 12, and FIG. 13.

The antibody or antibody fragment can be encoded by light and heavy chain variable nucleotide sequences according to clone-paired sequences selected from Table 1, can be encoded by light and heavy chain variable nucleotide sequences having at least 70%, 80%, or 90% identity to clone-paired sequences selected from Table 1, or can be encoded by light and heavy chain variable nucleotide sequences having at least 95% identity to clone-paired sequences selected from Table 1. The antibody or antibody fragment can comprise a light chain variable sequence and a heavy chain variable sequence selected from clone-paired sequences of Table 2, can comprise a light chain variable sequence and a heavy chain variable sequence having at least 70%, 80% or 90% identity to clone-paired sequences selected from Table 2, or can comprise a light chain variable sequence and a heavy chain variable sequence having 95% identity to clone-paired sequences selected from Table 2.

The antibody fragment can be a recombinant scFv (single chain fragment variable) antibody, Fab fragment, F(abø)₂ fragment, or Fv fragment. The antibody can be a chimeric antibody, or a bispecific antibody. The antibody can be an IgG, or a recombinant IgG antibody or antibody fragment comprising a mutated Fc portion. The mutated Fc portion can alter, eliminate or enhance FcR interactions; increase half-life; increase therapeutic efficacy; or a combination thereof. The mutated Fc portion can comprise a LALA mutation, a N297 mutation, a GASD/ALIE mutation, a YTE mutation, or an LS mutation. The mutated Fc portion can be glycan modified. The glycan modification can alter, eliminate, or enhance FcR interactions. The glycan modification can comprise an enzymatic or chemical addition or removal of glycans, or expression in a cell line engineered with a defined glycosylating pattern. The antibody or antibody fragment can further comprise a cell penetrating peptide and/or is an intrabody.

The use of the word “a” or “an” when used in conjunction with the term “comprising” in the claims and/or the specification can mean “one,” but it is also consistent with the meaning of “one or more,” “at least one,” and “one or more than one.” The word “about” is used herein to mean approximately, roughly, around, or in the region of. When the term “about” is used in conjunction with a numerical range, it modifies that range by extending the boundaries above and below the numerical values set forth. The term “about” can mean plus or minus 5% of the stated number.

Any method or composition described herein can be implemented with respect to any other method or composition described herein. Other objects, features and advantages of the present invention will become apparent from the following detailed description. It should be understood, however, that the detailed description and the specific examples, while indicating specific embodiments of the disclosure, are given by way of illustration only, since various changes and modifications within the spirit and scope of the disclosure will become apparent to those skilled in the art from this detailed description.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The following drawings form part of the present specification and are included to further demonstrate certain aspects of the present invention. The disclosure can be better understood by reference to one or more of these drawings in combination with the detailed description of specific embodiments presented herein.

FIG. 1 shows ELISA data of antibody binding to wells coated with the peptides Fba (SEQ ID NO: 40), or Met6 (SEQ ID NO: 38), or buffer for sera samples from ten different human donors (L70.S, L10.S, L56.S, C22-1, C06-1, C07-3, C14-2, L57.S, C-14-1, S-079). Positive controls included 1.10C (anti-Met6; SEQ ID NO: 12 and SEQ ID NO: 13) and 1.11D (anti-Fba; SEQ ID NO: 10 and SEQ ID NO: 11).

FIG. 2 shows ELISA inhibition data for the antibody 1.10C (anti-MFT6; SEQ ID NO: 12 and SEQ ID NO: 13), using the synthetic MET6 peptide (SEQ ID NO: 38) as an inhibitor to determine the reaction and binding affinity of 1.10C with the MET6 peptide. Each point is the mean of three determinations, and the data shown are from a typical experiment of four independent experiments.

FIG. 3 shows ELISA inhibition data for the antibody 1.11D (anti-Fba; SEQ ID NO: 10 and SEQ ID NO: 11), using the synthetic Fba peptide (SEQ ID NO: 40) as an inhibitor to determine the reaction and binding affinity of 1.11D with the Fba peptide. Each point is the mean of three determinations, and the data shown are from a typical experiment of four independent experiments.

FIG. 4 shows the determination of kinetic affinity constants of the antibodies 1.10C (right panel) and 1.11D (left panel) for binding to their cognate biotinylated peptides (MET6-Biotin, SEQ ID NO: 39; Fba-Biotin, SEQ ID NO: 41) as determined by bio-layer interferometry.

FIG. 5 shows the determination of steady-state affinity constants of the antibodies 1.10C (right panel) and 1.11D (left panel) for binding to their cognate biotinylated peptides (MET6-Biotin, SEQ ID NO: 39; Fba-Biotin, SEQ ID NO: 41) determined by bio-layer interferometry.

FIG. 6 demonstrates that the antibody 1.11D (anti-Fba; SEQ ID NO: 10 and SEQ ID NO: 11) specifically binds to whole length recombinant Fba proteins from both C. albicans (Top) and C. auris (Bottom) utilizing bio-layer interferometry. The antibody 1.10C (anti-MET6; SEQ ID NO: 12 and SEQ ID NO: 13) was used as a negative control.

FIG. 7 demonstrates that the antibody 1.10C (anti-MET6; SEQ ID NO: 12 and SEQ ID NO: 13) specifically binds to whole length recombinant MET6 protein from both C. albicans utilizing bio-layer interferometry. The antibody 1.11D (anti-Fba; SEQ ID NO: 10 and SEQ ID NO: 11) was used as a negative control.

FIG. 8 demonstrates that delivery by passive transfer of MAbs 1.10C (anti-MET6; SEQ ID NO: 12 and SEQ ID NO: 13) and 1.11D (anti-Fba; SEQ ID NO: 10 and SEQ ID NO: 11) confer protection against death by C. albicans. C57B/L6 mice were given an i.p. dose of either antibody singly or in combination four hours prior to hematogenous challenge with a lethal dose of C. albicans 3153A cells. Fluconazole™ (FLC) was used as a positive control and phosphate buffered saline (DPBS) was used as a negative control.

FIG. 9 demonstrates that delivery by passive transfer of a cocktail comprising MAbs 1.10C (anti-MET6; SEQ ID NO: 12 and SEQ ID NO: 13) and 1.11D (anti-Fba; SEQ ID NO: 10 and SEQ ID NO: 11) confers protection against death by C. auris. A/J mice were given an i.p. dose of either antibody singly or in combination four hours prior to hematogenous challenge with a lethal dose of C. auris cells. Fluconazole™ (FLC) was used as a positive control and phosphate buffered saline (DPBS) was used as a negative control.

FIGS. 10-11. Protein modeling for Met6 antibody 2B10.

FIGS. 12-13. Protein modeling for Fba antibody 2B10.

### DESCRIPTION OF ILLUSTRATIVE EMBODIMENTS

As discussed above, the present disclosure relates to antibodies binding to and neutralizing Candida and methods for use thereof.

These and other aspects of the disclosure are described in detail below.

Detailed descriptions of one or more preferred embodiments are provided herein. It is to be understood, however, that the present invention may be embodied in various forms. Therefore, specific details disclosed herein are not to be interpreted as limiting, but rather as a basis for the claims and as a representative basis for teaching one skilled in the art to employ the present invention in any appropriate manner.

The singular forms “a,” “an,” and “the” include plural reference unless the context clearly dictates otherwise.

Wherever any of the phrases “for example,” “such as,” “including” and the like are used herein, the phrase “and without limitation” is understood to follow unless explicitly stated otherwise. Similarly, “an example,” “exemplary” and the like are understood to be nonlimiting.

The term “substantially” allows for deviations from the descriptor that do not negatively impact the intended purpose. Descriptive terms are understood to be modified by the term “substantially” even if the word “substantially” is not explicitly recited.

The terms “comprising” and “including” and “having” and “involving” (and similarly “comprises”, “includes,” “has,” and “involves”) and the like are used interchangeably and have the same meaning. Specifically, each of the terms is defined consistent with the common United States patent law definition of “comprising” and is therefore interpreted to be an open term meaning “at least the following,” and is also interpreted not to exclude additional features, limitations, aspects, etc. Thus, for example, “a process involving steps a, b, and c” means that the process includes at least steps a, b and c. Wherever the terms “a” or “an” are used, “one or more” is understood, unless such interpretation is nonsensical in context.

As used interchangeably herein, “subject,” “individual,” or “patient,” can refer to a vertebrate, preferably a mammal, more preferably a human. In certain embodiments, “subject,” individual,” or “patient” refers to a reptile. Mammals include, but are not limited to, murines, simians, humans, farm animals, sport animals, and pets. The term “pet” includes a dog, cat, guinea pig, mouse, rat, rabbit, ferret, snake, turtle, lizard, bird, and the like. The term farm animal includes a horse, sheep, goat, chicken, pig, cow, donkey, llama, alpaca, turkey, and the like.

The terms “sample” or “biological sample” can refer to tissues, cells and biological fluids isolated from a subject, as well as tissues, cells and fluids present within a subject. Included within the usage of the terms “sample” or “biological sample”, therefore, is blood and a fraction or component of blood including blood serum, blood plasma, or lymph. “Sample” or “biological sample” can further include sputum, tears, saliva, mucous or serum, semen, cervical or vaginal secretions, amniotic fluid, placental tissues, urine, exudate, transudate, tissue scrapings, or feces.

### I. CANDIDA AND CANDIDIASIS

A. Candida spp.

Candida is a genus of yeasts and is the most common cause of fungal infections worldwide. Many species are harmless commensals or endosymbionts of hosts including humans; however, when mucosal barriers are disrupted, or the immune system is compromised they can invade and cause disease, known as an opportunistic infection. Candida albicans is the most commonly isolated species and can cause infections (candidiasis or thrush) in humans and other animals. In winemaking, some species of Candida can spoil wines.

Many species are found in gut flora, including C. albicans in mammalian hosts, whereas others live as endosymbionts in insect hosts. Systemic infections of the bloodstream and major organs (candidemia or invasive candidiasis), particularly in patients with an impaired immune system (immunocompromised), affect over 90,000 people a year in the U.S.

Antibiotics promote yeast (fungal) infections, including gastrointestinal (GI) Candida overgrowth and penetration of the GI mucosa. While women are more susceptible to genital yeast infections, men can also be infected. Certain factors, such as prolonged antibiotic use, increase the risk for both men and women. People with diabetes or the immunocompromised, such as those infected with HIV, are more susceptible to yeast infections.

When grown in a laboratory, Candida appears as large, round, white or cream colonies, which emit a yeasty odor on agar plates at room temperature. C. albicans ferments glucose and maltose to acid and gas, sucrose to acid, and does not ferment lactose, which helps to distinguish it from other Candida species.

Recent molecular phylogenetic studies show that the genus Candida is extremely polyphyletic (encompassing distantly-related species that do not form a natural group). Before the advent of inexpensive molecular methods, yeasts that were isolated from infected patients were often called Candida without clear evidence of relationship to other Candida species. For example, Candida glabrata, Candida guilliermondii, and Candida lusitaniae are clearly misclassified and will be placed in other genera once phylogenetic reorganization is complete.

Some species of Candida use a non-standard genetic code in the translation of their nuclear genes into the amino acid sequences of polypeptides. The difference in the genetic code between species possessing this alternative code is that the codon CUG (normally encoding the amino acid leucine) is translated by the yeast as a different amino acid, serine. The alternative translation of the CUG codon in these species is due to a nucleic acid sequence in the serine-tRNA (ser-tRNACAG), which has a guanosine located at position 33, 5′ to the anticodon. In all other tRNAs, this position is normally occupied by a pyrimidine (often uridine). This genetic code change is the only such known alteration in cytoplasmic mRNA, in both the prokaryotes, and the eukaryotes, involving the reassignment of a sense codon. This genetic code can be a mechanism for more rapid adaptation to the organism's environment, as well as playing an important role in the evolution of the genus Candida by creating genetic barriers that encouraged speciation.

Candida are almost universal in low numbers on healthy adult skin and C. albicans is part of the normal flora of the mucous membranes of the respiratory, gastrointestinal and female genital tracts. The dryness of skin compared to other tissues prevents the growth of the fungus, but damaged skin or skin in intertriginous regions is more amenable to rapid growth.

Overgrowth of several species, including C. albicans, can cause infections ranging from superficial, such as oropharyngeal candidiasis (thrush) or vulvovaginal candidiasis (vaginal candidiasis) and subpreputial candidiasis which may cause balanitis; to systemic, such as fungemia and invasive candidiasis. Oral candidiasis is common in elderly denture-wearers. In otherwise healthy individuals, these infections can be cured with topical or systemic antifungal medications (commonly over-the-counter antifungal treatments like miconazole or clotrimazole). In debilitated or immunocompromised patients, or if introduced intravenously (into the bloodstream), candidiasis may become a systemic disease producing abscesses, thrombophlebitis, endocarditis, or infections of the eyes or other organs. Typically, relatively severe neutropenia (low neutrophils) is a prerequisite for Candida to pass through the defenses of the skin and cause disease in deeper tissues; in such cases, mechanical disruption of the infected skin sites is typically a factor in the fungal invasion of the deeper tissues.

Among Candida species, C. albicans, which is a normal constituent of the human flora, a commensal of the skin and the gastrointestinal and genitourinary tracts, is responsible for the majority of Candida bloodstream infections (candidemia). Yet, there is an increasing incidence of infections caused by C. glabrata and C. rugosa, which could be because they are frequently less susceptible to the currently used azole-group of antifungals. Other medically important species include C. parapsilosis, C. tropicalis, C. auris and C. dubliniensis. Candida species, such as C. oleophila have been used as biological control agents in fruit.

B. Candidiasis

Candidiasis is a fungal infection due to any type of Candida (a type of yeast). When it affects the mouth, it is commonly called thrush. Signs and symptoms include white patches on the tongue or other areas of the mouth and throat. Other symptoms may include soreness and problems swallowing. When it affects the vagina, it is commonly called a yeast infection. Signs and symptoms include genital itching, burning, and sometimes a white “cottage cheese-like” discharge from the vagina. Yeast infections of the penis are less common and typically present with an itchy rash. Very rarely, yeast infections may become invasive, spreading to other parts of the body. This may result in fevers along with other symptoms depending on the parts involved.

More than 20 types of Candida can cause infection with Candida albicans being the most common. Infections of the mouth are most common among children less than one month old, the elderly, and those with weak immune systems. Conditions that result in a weak immune system include HIV/AIDS, the medications used after organ transplantation, diabetes, and the use of corticosteroids. Other risks include dentures and following antibiotic therapy. Vaginal infections occur more commonly during pregnancy, in those with weak immune systems, and following antibiotic use. Individuals at risk for invasive candidiasis include low birth weight babies, people recovering from surgery, people admitted to intensive care units, and those with an otherwise compromised immune systems.

Efforts to prevent infections of the mouth include the use of chlorhexidine mouth wash in those with poor immune function and washing out the mouth following the use of inhaled steroids. Little evidence supports probiotics for either prevention or treatment even among those with frequent vaginal infections. For infections of the mouth, treatment with topical clotrimazole or nystatin is usually effective. By mouth or intravenous fluconazole, itraconazole, or amphotericin B can be used if these do not work. A number of topical antifungal medications can be used for vaginal infections including clotrimazole. In those with widespread disease, an echinocandin such as caspofungin or micafungin is used. A number of weeks of intravenous amphotericin B can be used as an alternative. In certain groups at very high risk, antifungal medications can be used preventatively.

Infections of the mouth occur in about 6% of babies less than a month old. About 20% of those receiving chemotherapy for cancer and 20% of those with AIDS also develop the disease. About three-quarters of women have at least one yeast infection at some time during their lives. Widespread disease is rare except in those who have risk factors.

Signs and symptoms of candidiasis vary depending on the area affected. Most candidal infections result in minimal complications such as redness, itching, and discomfort, though complications may be severe or even fatal if left untreated in certain populations. In healthy (immunocompetent) persons, candidiasis is usually a localized infection of the skin, fingernails or toenails (onychomycosis), or mucosal membranes, including the oral cavity and pharynx (thrush), esophagus, and the genitalia (vagina, penis, etc.); less commonly in healthy individuals, the gastrointestinal tract, urinary tract, and respiratory tract are sites of Candida infection.

In immunocompromised individuals, Candida infections in the esophagus occur more frequently than in healthy individuals and have a higher potential of becoming systemic, causing a much more serious condition, a fungemia called candidemia. Symptoms of esophageal candidiasis include difficulty swallowing, painful swallowing, abdominal pain, nausea, and vomiting.

Thrush is commonly seen in infants. It is not considered abnormal in infants unless it lasts longer than a few weeks.

Infection of the vagina or vulva may cause severe itching, burning, soreness, irritation, and a whitish or whitish-gray cottage cheese-like discharge. Symptoms of infection of the male genitalia (balanitis thrush) include red skin around the head of the penis, swelling, irritation, itchiness and soreness of the head of the penis, thick, lumpy discharge under the foreskin, unpleasant odor, difficulty retracting the foreskin (phimosis), and pain when passing urine or during sex.

Common symptoms of gastrointestinal candidiasis in healthy individuals are anal itching, belching, bloating, indigestion, nausea, diarrhea, gas, intestinal cramps, vomiting, and gastric ulcers. Perianal candidiasis can cause anal itching; the lesion can be erythematous, papular, or ulcerative in appearance, and it is not considered to be a sexually transmissible disease. Abnormal proliferation of the candida in the gut may lead to dysbiosis. While it is not yet clear, this alteration may be the source of symptoms generally described as the irritable bowel syndrome, and other gastrointestinal diseases.

Candida yeasts are generally present in healthy humans, frequently part of the human body's normal oral and intestinal flora, and particularly on the skin; however, their growth is normally limited by the human immune system and by competition of other microorganisms, such as bacteria occupying the same locations in the human body. Candida requires moisture for growth, notably on the skin. For example, wearing wet swimwear for long periods of time is believed to be a risk factor. In extreme cases, superficial infections of the skin or mucous membranes may enter into the bloodstream and cause systemic Candida infections.

Factors that increase the risk of candidiasis include HIV/AIDS, mononucleosis, cancer treatments, steroids, stress, antibiotic usage, diabetes, and nutrient deficiency. Hormone replacement therapy and infertility treatments may also be predisposing factors. Treatment with antibiotics can lead to eliminating the yeast's natural competitors for resources in the oral and intestinal flora; thereby increasing the severity of the condition. A weakened or undeveloped immune system or metabolic illnesses are significant predisposing factors of candidiasis. Almost 15% of people with weakened immune systems develop a systemic illness caused by Candida species. Diets high in simple carbohydrates have been found to affect rates of oral candidiases.

C. albicans was isolated from the vaginas of 19% of apparently healthy women, i.e., those who experienced few or no symptoms of infection. External use of detergents or douches or internal disturbances (hormonal or physiological) can perturb the normal vaginal flora, consisting of lactic acid bacteria, such as lactobacilli, and result in an overgrowth of Candida cells, causing symptoms of infection, such as local inflammation. Pregnancy and the use of oral contraceptives have been reported as risk factors. Diabetes mellitus and the use of antibiotics are also linked to increased rates of yeast infections.

In penile candidiasis, the causes include sexual intercourse with an infected individual, low immunity, antibiotics, and diabetes. Male genital yeast infections are less common, but a yeast infection on the penis caused from direct contact via sexual intercourse with an infected partner is not uncommon.

Symptoms of vaginal candidiasis are also present in the more common bacterial vaginosis; aerobic vaginitis is distinct and should be excluded in the differential diagnosis. In a 2002 study, only 33% of women who were self-treating for a yeast infection actually had such an infection, while most had either bacterial vaginosis or a mixed-type infection.

Diagnosis of a yeast infection is done either via microscopic examination or culturing. For identification by light microscopy, a scraping or swab of the affected area is placed on a microscope slide. A single drop of 10% potassium hydroxide (KOH) solution is then added to the specimen. The KOH dissolves the skin cells, but leaves the Candida cells intact, permitting visualization of pseudohyphae and budding yeast cells typical of many Candida species.

For the culturing method, a sterile swab is rubbed on the infected skin surface. The swab is then streaked on a culture medium. The culture is incubated at 37° C. (98.6° F.) for several days, to allow development of yeast or bacterial colonies. The characteristics (such as morphology and colour) of the colonies may allow initial diagnosis of the organism causing disease symptoms.

Respiratory, gastrointestinal, and esophageal candidiasis require an endoscopy to diagnose. For gastrointestinal candidiasis, it is necessary to obtain a 3-5 milliliter sample of fluid from the duodenum for fungal culture. The diagnosis of gastrointestinal candidiasis is based upon the culture containing in excess of 1,000 colony-forming units per milliliter. Candidiasis can be divided into these types:

Mucosal Candidiasis
Oral candidiasis (thrush, oropharyngeal candidiasis)
   Pseudomembranous candidiasis
   Erythematous candidiasis
   Hyperplastic candidiasis
   Denture-related stomatitis—Candida organisms are involved in about 90% of cases
   Angular cheilitis—Candida species are responsible for about 20% of cases, mixed infection of C. albicans and Staphylococcus aureus for about 60% of cases.
   Median rhomboid glossitis
Candidal vulvovaginitis (vaginal yeast infection)
Candidal balanitis—infection of the glans penis, almost exclusively occurring in uncircumcised males
Esophageal candidiasis (candidal esophagitis)
Gastrointestinal candidiasis
Respiratory candidiasis

Cutaneous Candidiasis
Candidial folliculitis
Candidal intertrigo
Candidal paronychia
Perianal candidiasis, may present as pruritus ani
Candidid
Chronic mucocutaneous candidiasis
Congenital cutaneous candidiasis
Diaper candidiasis: an infection of a child's diaper area
Erosio interdigitalis blastomycetica
Candidial onychomycosis (nail infection) caused by Candida

Systemic Candidiasis
Candidemia, a form of fungemia which may lead to sepsis
Invasive candidiasis (disseminated candidiasis)—organ infection by Candida
Chronic systemic candidiasis (hepatosplenic candidiasis)—sometimes arises during recovery from neutropenia

Antibiotic Candidiasis (Iatrogenic Candidiasis)

A diet that supports the immune system and is not high in simple carbohydrates contributes to a healthy balance of the oral and intestinal flora. While yeast infections are associated with diabetes, the level of blood sugar control may not affect the risk. Wearing cotton underwear may help to reduce the risk of developing skin and vaginal yeast infections, along with not wearing wet clothes for long periods of time.

Oral hygiene can help prevent oral candidiasis when people have a weakened immune system. For people undergoing cancer treatment, chlorhexidine mouthwash can prevent or reduce thrush. People who use inhaled corticosteroids can reduce the risk of developing oral candidiasis by rinsing the mouth with water or mouthwash after using the inhaler.

For women who experience recurrent yeast infections, there is limited evidence that oral or intravaginal probiotics help to prevent future infections. This includes either as pills or as yogurt.

Candidiasis is treated with antifungal medications; these include clotrimazole, nystatin, fluconazole, voriconazole, amphotericin B, and echinocandins. Intravenous fluconazole or an intravenous echinocandin such as caspofungin are commonly used to treat immunocompromised or critically ill individuals.

The 2016 revision of the clinical practice guideline for the management of candidiasis lists a large number of specific treatment regimens for Candida infections that involve different Candida species, forms of antifungal drug resistance, immune statuses, and infection localization and severity. Gastrointestinal candidiasis in immunocompetent individuals is treated with 100-200 mg fluconazole per day for 2-3 weeks.

Mouth and throat candidiasis are treated with antifungal medication. Oral candidiasis usually responds to topical treatments; otherwise, systemic antifungal medication may be needed for oral infections. Candidal skin infections in the skin folds (candidal intertrigo) typically respond well to topical antifungal treatments (e.g., nystatin or miconazole). Systemic treatment with antifungals by mouth is reserved for severe cases or if treatment with topical therapy is unsuccessful. Candida esophagitis may be treated orally or intravenously; for severe or azole-resistant esophageal candidiasis, treatment with amphotericin B may be necessary.

Vaginal yeast infections are typically treated with topical antifungal agents. A one-time dose of fluconazole is 90% effective in treating a vaginal yeast infection. For severe nonrecurring cases, several doses of fluconazole is recommended. Local treatment can include vaginal suppositories or medicated douches. Other types of yeast infections require different dosing. Gentian violet can be used for thrush in breastfeeding babies. C. albicans can develop resistance to fluconazole, this being more of an issue in those with HIV/AIDS who are often treated with multiple courses of fluconazole for recurrent oral infections.

For vaginal yeast infection in pregnancy, topical imidazole or triazole antifungals are considered the therapy of choice owing to available safety data. Systemic absorption of these topical formulations is minimal, posing little risk of transplacental transfer. In vaginal yeast infection in pregnancy, treatment with topical azole antifungals is recommended for 7 days instead of a shorter duration. No benefit from probiotics has been found for active infections.

Systemic candidiasis occurs when Candida yeast enters the bloodstream and may spread (becoming disseminated candidiasis) to other organs, including the central nervous system, kidneys, liver, bones, muscles, joints, spleen, or eyes. Treatment typically consists of oral or intravenous antifungal medications. In candidal infections of the blood, intravenous fluconazole or an echinocandin such as caspofungin can be used. Amphotericin B is another option.

### II. MONOCLONAL ANTIBODIES AND PRODUCTION THEREOF

As used herein, an “antibody” or “antigen-binding polypeptide” can refer to a polypeptide or a polypeptide complex that specifically recognizes and binds to an antigen. An antibody can be a whole antibody and any antigen binding fragment or a single chain thereof. For example, “antibody” can include any protein or peptide containing molecule that comprises at least a portion of an immunoglobulin molecule having biological activity of binding to the antigen. Non-limiting examples a complementarity determining region (CDR) of a heavy or light chain or a ligand binding portion thereof a heavy chain or light chain variable region, a heavy chain or light chain constant region, a framework (FR) region, or any portion thereof, or at least one portion of a binding protein. As used herein, the term “antibody” can refer to an immunoglobulin molecule and immunologically active portions of an immunoglobulin (Ig) molecule, i.e., a molecule that contains an antigen binding site that specifically binds (immunoreacts with) an antigen. By “specifically binds” or “immunoreacts with” is meant that the antibody reacts with one or more antigenic determinants of the desired antigen and does not react with other polypeptides.

The terms “antibody fragment” or “antigen-binding fragment”, as used herein, is a portion of an antibody such as F_{(ab′)2}, F_{(ab)2}, F_{ab}′, F_{ab}, Fv, scFv and the like. Regardless of structure, an antibody fragment binds with the same antigen that is recognized by the intact antibody. The term “antibody fragment” can include aptamers, minibodies, and diabodies. The term “antibody fragment” can also include any synthetic or genetically engineered protein that acts like an antibody by binding to a specific antigen to form a complex. Antibodies, antigen-binding polypeptides, variants, or derivatives described herein include, but are not limited to, polyclonal, monoclonal, multispecific, human, humanized or chimeric antibodies, single chain antibodies, epitope-binding fragments, e.g., Fab, Fab′ and F(ab′)₂, Fd, Fvs, single-chain Fvs (scFv), single-chain antibodies, dAb (domain antibody), minibodies, disulfide-linked Fvs (sdFv), fragments comprising either a VL or VH domain, fragments produced by a Fab expression library, and anti-idiotypic (anti-Id) antibodies.

A “single-chain variable fragment” or “scFv” can refer to a fusion protein of the variable regions of the heavy (V_{H}) and light chains (V_{L}) of immunoglobulins. A single chain Fv (“scFv”) polypeptide molecule is a covalently linked VH:VL heterodimer, which can be expressed from a gene fusion including VH- and VL-encoding genes linked by a peptide-encoding linker. See Huston et al., Proc. Nat'l Acad. Sci. USA 85(16):5879-5883 (1988). In some aspects, the regions are connected with a short linker peptide of ten to about 25 amino acids. The linker can be rich in glycine for flexibility, as well as serine or threonine for solubility, and can either connect the N-terminus of the V_{H} with the C-terminus of the V_{L}, or vice versa. This protein retains the specificity of the original immunoglobulin, despite removal of the constant regions and the introduction of the linker. A number of methods have been described to discern chemical structures for converting the naturally aggregated, but chemically separated, light and heavy polypeptide chains from an antibody V region into an scFv molecule, which will fold into a three-dimensional structure substantially similar to the structure of an antigen-binding site. See, U.S. Pat. Nos. 5,091,513; 5,892,019; 5,132,405; and 4,946,778, each of which are incorporated by reference in their entireties.

Aspects of the invention provide isolated monoclonal antibodies. The term “isolated” as used herein with respect to cells and nucleic acids, such as DNA or RNA, can refer to molecules separated from other DNAs or RNAs, respectively, that are present in the natural source of the macromolecule. The term “isolated” can also refer to a nucleic acid or peptide that is substantially free of cellular material, viral material, or culture medium when produced by recombinant DNA techniques, or chemical precursors or other chemicals when chemically synthesized. For example, an “isolated nucleic acid” can include nucleic acid fragments which are not naturally occurring as fragments and would not be found in the natural state. “Isolated” can also refer to cells or polypeptides which are isolated from other cellular proteins or tissues. Isolated polypeptides can include both purified and recombinant polypeptides. For example, an “isolated antibody” can be one that has been separated and/or recovered from a component of its natural environment. Contaminant components of its natural environment are materials that would interfere with diagnostic or therapeutic uses for the antibody, and can include enzymes, hormones, and other proteinaceous or non-proteinaceous solutes. In particular embodiments, the antibody is purified: (1) to greater than 95% by weight of antibody as determined by the Lowry method, and most particularly more than 99% by weight; (2) to a degree sufficient to obtain at least 15 residues of N-terminal or internal amino acid sequence by use of a spinning cup sequenator; or (3) to homogeneity by SDS-PAGE under reducing or non-reducing conditions using Coomassie blue or silver stain. Isolated antibody includes the antibody in situ within recombinant cells since at least one component of the antibody's natural environment will not be present. Ordinarily, however, isolated antibody will be prepared by at least one purification step.

The basic four-chain antibody unit is a heterotetrameric glycoprotein composed of two identical light (L) chains and two identical heavy (H) chains. An IgM antibody consists of 5 basic heterotetramer units along with an additional polypeptide called J chain, and therefore contain 10 antigen binding sites, while secreted IgA antibodies can polymerize to form polyvalent assemblages comprising 2-5 of the basic 4-chain units along with J chain. In the case of IgGs, the 4-chain unit is generally about 150,000 daltons. Each L chain is linked to an H chain by one covalent disulfide bond, while the two H chains are linked to each other by one or more disulfide bonds depending on the H chain isotype. Each H and L chain also has regularly spaced intrachain disulfide bridges. Each H chain has at the N-terminus, a variable region (V_{H}) followed by three constant domains (C_{H}) for each of the alpha and gamma chains and four C_{H} domains for mu and isotypes. Each L chain has at the N-terminus, a variable region (V_{L}) followed by a constant domain (C_{L}) at its other end. The V_{L} is aligned with the V_{H} and the C_{L} is aligned with the first constant domain of the heavy chain (C_{H1}). Particular amino acid residues are believed to form an interface between the light chain and heavy chain variable regions. The pairing of a V_{H} and V_{L} together forms a single antigen-binding site. For the structure and properties of the different classes of antibodies, see, e.g., Basic and Clinical immunology, 8th edition, Daniel P. Stites, Abba I. Terr and Tristram G. Parslow (eds.), Appleton & Lange, Norwalk, Conn., 1994, page 71, and Chapter 6.

The L chain from any vertebrate species can be assigned to one of two clearly distinct types, called kappa and lambda based on the amino acid sequences of their constant domains (C_{L}). Depending on the amino acid sequence of the constant domain of their heavy chains (C_{H}), immunoglobulins can be assigned to different classes or isotypes. There are five classes of immunoglobulins: IgA, IgD, IgE, IgG, and IgM, having heavy chains designated alpha, delta, epsilon, gamma and mu, respectively. They gamma and alpha classes are thrther divided into subclasses on the basis of relatively minor differences in C_{H} sequence and function, humans express the following subclasses: IgG1, IgG2, IgG3, IgG4, IgA1, and IgA2.

The term “variable” can refer to the fact that certain segments of the V domains differ extensively in sequence among antibodies. The V domain mediates antigen binding and provides specificity of a particular antibody for its particular antigen. However, the variability is not evenly distributed across the 110-amino acid span of the variable regions. Instead, the V regions consist of relatively invariant stretches called framework regions (FRs) of 15-30 amino acids separated by shorter regions of extreme variability called “hypervariable regions” that are each 9-12 amino acids long. The variable regions of native heavy and light chains each comprise four FRs, largely adopting a beta-sheet configuration, connected by three hypervariable regions, which form loops connecting, and in some cases forming part of, the beta-sheet structure. The hypervariable regions in each chain are held together in close proximity by the FRs and, with the hypervariable regions from the other chain, contribute to the formation of the antigen-binding site of antibodies (see Kabat et al., Sequences of Proteins of Immunological Interest, 5th Ed. Public Health Service, National institutes of Health, Bethesda, Md. (1991)). The constant domains are not involved directly in binding an antibody to an antigen, but exhibit various effector functions, such as participation of the antibody in antibody dependent cellular cytotoxicity (ADCC), antibody-dependent cellular phagocytosis (ADCP), antibody-dependent neutrophil phagocytosis (ADNP), and antibody-dependent complement deposition (ADCD).

The term hypervariable region” when used herein can refer to the amino acid residues of an antibody that are responsible for antigen binding. The hypervariable region generally comprises amino acid residues from a “complementarity determining region” or “CDR,” e.g., around about residues 24-34 (L1), 50-56 (L2) and 89-97 (L3) in the V_{L}, and around about 31-35 (H1), 50-65 (H2) and 95-102 (H3) in the V_{H} when numbered in accordance with the Kabat numbering system; Kabat et al., Sequences of Proteins of Immunological Interest, 5th Ed. Public Health Service, National Institutes of Health, Bethesda, Md. (1991); and/or those residues from a “hypervariable loop” (e.g., residues 24-34 (L1), 50-56 (L2) and 89-97 (L3) in the V_{L}, and 26-32 (H1), 52-56 (H2) and 95-101 (H3) in the V_{H} when numbered in accordance with the Chothia numbering system; Chothia and Lesk, J. Mol. Biol. 196:901-917 (1987); and/or those residues from a “hypervariable loop”/CDR (e.g., residues 27-38 (L1), 56-65 (L2) and 105-120 (L3) in the V_{L}, and 27-38 (H1), 56-65 (H2) and 105-120 (H3) in the V_{H} when numbered in accordance with the IMGT numbering system; Lefranc et al., Nucl. Acids Res. 27:209-212 (1999), Ruiz et al., Nucl. Acids Res. 28:219-221 (2000). Optionally, the antibody has symmetrical insertions at one or more of the following points 28, 36 (L1) 63, 74-75 (L2) and 123 (L3) in the V_{L}, and 28, 36 (H1), 63, 74-75 (H2) and 123 (H3) in the V_{sub}H when numbered in accordance with AHo; Honneger, A. and Plunkthun, A., J. Mol. Biol. 309:657-670 (2001).

By “germline nucleic acid residue” is meant the nucleic acid residue that naturally occurs in a germline gene encoding a constant or variable region. “Germline gene” is the DNA found in a germ cell (i.e., a cell destined to become an egg or in the sperm). A “germline mutation” refers to a heritable change in a particular DNA that has occurred in a germ cell or the zygote at the single-cell stage, and when transmitted to offspring, such a mutation is incorporated in every cell of the body. A germline mutation is in contrast to a somatic mutation which is acquired in a single body cell. In some cases, nucleotides in a germline DNA sequence encoding for a variable region are mutated (i.e., a somatic mutation) and replaced with a different nucleotide.

The term “monoclonal antibody” as used herein can refer to an antibody obtained from a population of substantially homogeneous antibodies, i.e., the individual antibodies comprising the population are identical except for possible naturally occurring mutations that can be present in minor amounts. Monoclonal antibodies are highly specific, being directed against a single antigenic site. Furthermore, in contrast to polyclonal antibody preparations that include different antibodies directed against different determinants (epitopes), each monoclonal antibody is directed against a single determinant on the antigen. In addition to their specificity, the monoclonal antibodies are advantageous in that they can be synthesized uncontaminated by other antibodies. The modifier “monoclonal” is not to be construed as requiring production of the antibody by any particular method. For example, the monoclonal antibodies useful in the present invention can be prepared by the hybridoma methodology first described by Kohler et al., Nature, 256:495 (1975), or can be made using recombinant DNA methods in bacterial, eukaryotic animal or plant cells (see, e.g., U.S. Pat. No. 4,816,567) after single cell sorting of an antigen specific B cell, an antigen specific plasmablast responding to an infection or immunization, or capture of linked heavy and light chains from single cells in a bulk sorted antigen specific collection. The “monoclonal antibodies” can also be isolated from phage antibody libraries using the techniques described in Clackson et al., Nature, 352:624-628 (1991) and Marks et al., J. Mol. Biol., 222:581-597 (1991), for example.

Fully human antibodies are antibody molecules in which the entire sequence of both the light chain and the heavy chain, including the CDRs, arise from human genes. Human monoclonal antibodies can be prepared, for example, by using the human B-cell hybridoma technique (see Kozbor et al., Immunol Today 4: 72, 1983); and the EBV hybridoma technique to produce human monoclonal antibodies (see Cole et al., In: MONOCLONAL ANTIBODIES AND CANCER THERAPY, Alan R. Liss, Inc., pp. 77-96, 1985). Human monoclonal antibodies can be utilized and can be produced by using human hybridomas (see Cote et al., Proc. Nat'l Acad. Sci. USA 80: 2026-2030, 1983) or by transforming human B-cells with Epstein Barr Virus in vitro (see Cole et al., In: MONOCLONAL ANTIBODIES AND CANCER THERAPY, Alan R. Liss, Inc., pp. 77-96, 1985).

In addition, human antibodies can also be produced using other techniques, including phage display libraries (xee Hoogenboom and Winter, J. Mol. Biol., 227:381, 1991; Marks et al., J. Mol. Biol., 222:581, 1991). Similarly, human antibodies can be made by introducing human immunoglobulin loci into transgenic animals, e.g., mice in which the endogenous immunoglobulin genes have been partially or completely inactivated. Upon challenge, human antibody production is observed, which closely resembles that seen in humans in all respects, including gene rearrangement, assembly, and antibody repertoire. This approach is described, for example, in U.S. Pat. Nos. 5,545,807; 5,545,806; 5,569,825; 5,625,126; 5,633,425; 5,661,016, and in Marks et al., Bio/Technology 10, 779-783 (1992); Lonberg et al., Nature 368 856-859 (1994); Morrison, Nature 368, 812-13 (1994); Fishwild et al., Nature Biotechnology 14, 845-51 (1996); Neuberger, Nature Biotechnology 14, 826 (1996); and Lonberg and Huszar, Intern. Rev. Immunol. 13 65-93 (1995).

Human antibodies can additionally be produced using transgenic nonhuman animals which are modified so as to produce fully human antibodies rather than the animal's endogenous antibodies in response to challenge by an antigen (see PCT publication WO94/02602 and U.S. Pat. No. 6,673,986). The endogenous genes encoding the heavy and light immunoglobulin chains in the nonhuman host have been incapacitated, and active loci encoding human heavy and light chain immunoglobulins are inserted into the host's genome. The human genes are incorporated, for example, using yeast artificial chromosomes containing the requisite human DNA segments. An animal which provides all the desired modifications is then obtained as progeny by crossbreeding intermediate transgenic animals containing fewer than the full complement of the modifications. The preferred embodiment of such a nonhuman animal is a mouse and is termed the Xenomouse™ as disclosed in PCT publications WO 96/33735 and WO 96/34096. This animal produces B cells which secrete fully human immunoglobulins. The antibodies can be obtained directly from the animal after immunization with an immunogen of interest, as, for example, a preparation of a polyclonal antibody, or alternatively from immortalized B cells derived from the animal, such as hybridomas producing monoclonal antibodies. Additionally, the genes encoding the immunoglobulins with human variable regions can be recovered and expressed to obtain the antibodies directly or can be further modified to obtain analogs of antibodies such as, for example, single chain Fv (scFv) molecules. In addition, companies such as Creative BioLabs (Shirley, N.Y.) can be engaged to provide human antibodies directed against a selected antigen using technology similar to that described herein.

A. General Methods

Monoclonal antibodies binding to Candida will have several applications. These include the production of diagnostic kits for use in detecting and diagnosing Candida infection, as well as for treating the same. In these contexts, one can link such antibodies to diagnostic or therapeutic agents, use them as capture agents or competitors in competitive assays, or use them individually without additional agents being attached thereto. The antibodies can be mutated or modified, as discussed further below. Methods for preparing and characterizing antibodies are well known in the art (see, e.g., Antibodies: A Laboratory Manual, Cold Spring Harbor Laboratory, 1988; U.S. Pat. No. 4,196,265).

The methods for generating monoclonal antibodies (MAbs) generally begin along the same lines as those for preparing polyclonal antibodies. The first step for both these methods is immunization of an appropriate host or identification of subjects who are immune due to prior natural infection or vaccination with a licensed or experimental vaccine. As is well known in the art, a given composition for immunization can vary in its immunogenicity. It is often necessary therefore to boost the host immune system, as can be achieved by coupling a peptide or polypeptide immunogen to a carrier. Exemplary and preferred carriers are keyhole limpet hemocyanin (KLH) and bovine serum albumin (BSA). Other albumins such as ovalbumin, mouse serum albumin or rabbit serum albumin can also be used as carriers. Means for conjugating a polypeptide to a carrier protein are well known in the art and include glutaraldehyde, m-maleimidobencoyl-N-hydroxysuccinimide ester, carbodiimyde and bis-biazotized benzidine. As also is well known in the art, the immunogenicity of a particular immunogen composition can be enhanced by the use of non-specific stimulators of the immune response, known as adjuvants. Exemplary and preferred adjuvants in animals include complete Freund's adjuvant (a non-specific stimulator of the immune response containing killed Mycobacterium tuberculosis), incomplete Freund's adjuvants and aluminum hydroxide adjuvant and in humans include alum, CpG, MFP59 and combinations of immunostimulatory molecules (“Adjuvant Systems”, such as AS01 or AS03). Additional experimental forms of inoculation to induce Candida-specific B cells can be conducted, including nanoparticle vaccines, or gene-encoded antigens delivered as DNA or RNA genes in a physical delivery system (such as lipid nanoparticle or on a gold biolistic bead), and delivered with needle, gene gun, transcutaneous electroporation device. The antigen gene also can be carried as encoded by a replication competent or defective viral vector such as adenovirus, adeno-associated virus, poxvirus, herpesvirus, or alphavirus replicon, or alternatively a virus-like particle.

In the case of human antibodies against natural pathogens, a suitable approach is to identify subjects that have been exposed to the pathogens, such as those who have been diagnosed as having contracted the disease, or those who have been vaccinated to generate protective immunity against the pathogen or to test the safety or efficacy of an experimental vaccine. Circulating anti-pathogen antibodies can be detected, and antibody encoding or producing B cells from the antibody-positive subject can then be obtained.

The amount of immunogen composition used in the production of polyclonal antibodies varies upon the nature of the immunogen as well as the animal used for immunization. A variety of routes can be used to administer the immunogen (subcutaneous, intramuscular, intradermal, intravenous and intraperitoneal). The production of polyclonal antibodies may be monitored by sampling blood of the immunized animal at various points following immunization. A second, booster injection, also can be given. The process of boosting and titering is repeated until a suitable titer is achieved. When a desired level of immunogenicity is obtained, the immunized animal can be bled and the serum isolated and stored, and/or the animal can be used to generate MAbs.

Following immunization, somatic cells that can produce antibodies, specifically B lymphocytes (B cells), are selected for use in the MAb generating protocol. These cells can be obtained from biopsied spleens, lymph nodes, tonsils or adenoids, bone marrow aspirates or biopsies, tissue biopsies from mucosal organs like lung or GI tract, or from circulating blood. The antibody-producing B lymphocytes from the immunized animal or immune human are then fused with cells of an immortal myeloma cell, generally one of the same species as the animal that was immunized or human or human/mouse chimeric cells. Myeloma cell lines suited for use in hybridoma-producing fusion procedures preferably are non-antibody-producing, have high fusion efficiency, and enzyme deficiencies that render then incapable of growing in certain selective media which support the growth of only the desired fused cells (hybridomas). Any one of a number of myeloma cells can be used, as are known to those of skill in the art (Goding, pp. 65-66, 1986; Campbell, pp. 75-83, 1984). HMMA2.5 cells or MFP-2 cells are particularly useful examples of such cells.

Methods for generating hybrids of antibody-producing spleen or lymph node cells and myeloma cells usually comprise mixing somatic cells with myeloma cells in a 2:1 proportion, though the proportion can vary from about 20:1 to about 1:1, respectively, in the presence of an agent or agents (chemical or electrical) that promote the fusion of cell membranes. In some cases, transformation of human B cells with Epstein Barr virus (EBV) as an initial step increases the size of the B cells, enhancing fusion with the relatively large-sized myeloma cells. Transformation efficiency by EBV is enhanced by using CpG and a Chk2 inhibitor drug in the transforming medium. Alternatively, human B cells can be activated by co-culture with transfected cell lines expressing CD40 Ligand (CD154) in medium containing additional soluble factors, such as IL-21 and human B cell Activating Factor (BAFF), a Type II member of the TNF superfamily. Fusion methods using Sendai virus have been described by Kohler and Milstein (1975; 1976), and those using polyethylene glycol (PEG), such as 37% (v/v) PEG, by Gefter et al. (1977). The use of electrically induced fusion methods also is appropriate (Goding, pp. 71-74, 1986) and there are processes for better efficiency (Yu et al, 2008). Fusion procedures usually produce viable hybrids at low frequencies, about 1×10⁻⁶ to 1×10⁻⁸, but with optimized procedures one can achieve fusion efficiencies close to 1 in 200 (Yu et al., 2008). However, relatively low efficiency of fusion does not pose a problem, as the viable, fused hybrids are differentiated from the parental, infused cells (particularly the infused myeloma cells that would normally continue to divide indefinitely) by culturing in a selective medium. The selective medium is generally one that contains an agent that blocks the de novo synthesis of nucleotides in the tissue culture medium. Exemplary and preferred agents are aminopterin, methotrexate, and azaserine. Aminopterin and methotrexate block de novo synthesis of both purines and pyrimidines, whereas azaserine blocks only purine synthesis. Where aminopterin or methotrexate is used, the medium is supplemented with hypoxanthine and thymidine as a source of nucleotides (HAT medium). Where azaserine is used, the medium is supplemented with hypoxanthine. Ouabain is added if the B cell source is an EBV-transformed human B cell line, in order to eliminate EBV-transformed lines that have not fused to the myeloma.

The preferred selection medium is HAT or HAT with ouabain. Only cells capable of operating nucleotide salvage pathways are able to survive in HAT medium. The myeloma cells are defective in key enzymes of the salvage pathway, e.g., hypoxanthine phosphoribosyl transferase (HPRT), and they cannot survive. The B cells can operate this pathway, but they have a limited life span in culture and generally die within about two weeks. Therefore, the only cells that can survive in the selective media are those hybrids formed from myeloma and B cells. When the source of B cells used for fusion is a line of EBV-transformed B cells, as here, ouabain can also be used for drug selection of hybrids as EBV-transformed B cells are susceptible to drug killing, whereas the myeloma partner used is chosen to be ouabain resistant.

Culturing provides a population of hybridomas from which specific hybridomas are selected. Typically, selection of hybridomas is performed by culturing the cells by single-clone dilution in microtiter plates, followed by testing the individual clonal supernatants (after about two to three weeks) for the desired reactivity. The assay should be sensitive, simple and rapid, such as radioimmunoassays, enzyme immunoassays, cytotoxicity assays, plaque assays dot immunobinding assays, and the like. The selected hybridomas are then serially diluted or single-cell sorted by flow cytometric sorting and cloned into individual antibody-producing cell lines, which clones can then be propagated indefinitely to provide mAbs. The cell lines can be exploited for MAb production in two basic ways. A sample of the hybridoma can be injected (often into the peritoneal cavity) into an animal (e.g., a mouse). Optionally, the animals are primed with a hydrocarbon, especially oils such as pristane (tetramethylpentadecane) prior to injection. When human hybridomas are used in this way, it is optimal to inject immunocompromised mice, such as SCID mice, to prevent tumor rejection. The injected animal develops tumors secreting the specific monoclonal antibody produced by the fused cell hybrid. The body fluids of the animal, such as serum or ascites fluid, can then be tapped to provide mAbs in high concentration. The individual cell lines could also be cultured in vitro, where the mAbs are naturally secreted into the culture medium from which they can be readily obtained in high concentrations. Alternatively, human hybridoma cells lines can be used in vitro to produce immunoglobulins in cell supernatant. The cell lines can be adapted for growth in serum-free medium to optimize the ability to recover human monoclonal immunoglobulins of high purity.

mAbs produced by either means can be further purified, if desired, using filtration, centrifugation and various chromatographic methods such as FPLC or affinity chromatography. Fragments of the monoclonal antibodies of the disclosure can be obtained from the purified monoclonal antibodies by methods which include digestion with enzymes, such as pepsin or papain, and/or by cleavage of disulfide bonds by chemical reduction. Alternatively, monoclonal antibody fragments encompassed by the present invention can be synthesized using an automated peptide synthesizer.

For example, a molecular cloning approach can be used to generate monoclonal antibodies. Single B cells labelled with the antigen of interest can be sorted physically using paramagnetic bead selection or flow cytometric sorting, then RNA can be isolated from the single cells and antibody genes amplified by RT-PCR. Alternatively, antigen-specific bulk sorted populations of cells can be segregated into microvesicles and the matched heavy and light chain variable genes recovered from single cells using physical linkage of heavy and light chain amplicons, or common barcoding of heavy and light chain genes from a vesicle. Matched heavy and light chain genes form single cells also can be obtained from populations of antigen specific B cells by treating cells with cell-penetrating nanoparticles bearing RT-PCR primers and barcodes for marking transcripts with one barcode per cell. The antibody variable genes also can be isolated by RNA extraction of a hybridoma line and the antibody genes obtained by RT-PCR and cloned into an immunoglobulin expression vector. Alternatively, combinatorial immunoglobulin phagemid libraries are prepared from RNA isolated from the cell lines and phagemids expressing appropriate antibodies are selected by panning using fungal antigens. The advantages of this approach over conventional hybridoma techniques are that approximately 10⁴ times as many antibodies can be produced and screened in a single round, and that new specificities are generated by H and L chain combination which further increases the chance of finding appropriate antibodies.

Other U.S. patents, each incorporated herein by reference, that teach the production of antibodies useful in the present invention include U.S. Pat. No. 5,565,332, which describes the production of chimeric antibodies using a combinatorial approach; U.S. Pat. No. 4,816,567 which describes recombinant immunoglobulin preparations; and U.S. Pat. No. 4,867,973 which describes antibody-therapeutic agent conjugates.

B. Antibodies of the Present Disclosure

Antibodies according to the present disclosure can be characterized, in the first instance, by their binding specificity. As used herein, the terms “immunological binding,” and “immunological binding properties” can refer to the non-covalent interactions of the type which occur between an immunoglobulin molecule and an antigen for which the immunoglobulin is specific. The strength, or affinity of immunological binding interactions can be expressed in terms of the equilibrium binding constant (K_{D}) of the interaction, wherein a smaller K_{D} represents a greater affinity. Those of skill in the art, by assessing the binding specificity/affinity of a given antibody using techniques well known to those of skill in the art, can determine whether such antibodies fall within the scope of the instant claims. For example, the epitope to which a given antibody binds can comprise a single contiguous sequence of 3 or more (e.g., 3, 4, 5, 6, 7, 8, 9, 10, 11, 12, 13, 14, 15, 16, 17, 18, 19, 20) amino acids located within the antigen molecule (e.g., a linear epitope in a domain). Alternatively, the epitope can comprise a plurality of non-contiguous amino acids (or amino acid sequences) located within the antigen molecule (e.g., a conformational epitope). As used herein, the term “epitope” can include any protein determinant capable of specific binding to an immunoglobulin, a scFv, or a T-cell receptor. The variable region allows the antibody to selectively recognize and specifically bind epitopes on antigens. For example, the VL domain and VH domain, or subset of the complementarity determining regions (CDRs), of an antibody combine to form the variable region that defines a three-dimensional antigen-binding site. This quaternary antibody structure forms the antigen-binding site present at the end of each arm of the Y. Epitopic determinants usually consist of chemically active surface groupings of molecules such as amino acids or sugar side chains and usually have specific three-dimensional structural characteristics, as well as specific charge characteristics. For example, antibodies can be raised against N- terminal or C-terminal peptides of a polypeptide. More specifically, the antigen-binding site is defined by three CDRs on each of the VH and VL chains (i.e., CDR-H1, CDR-H2, CDR-H3, CDR-L1, CDR-L2 and CDR-L3).

Various techniques known to persons of ordinary skill in the art can be used to determine whether an antibody “interacts with one or more amino acids” within a polypeptide or protein. Exemplary techniques include, for example, routine cross-blocking assays, such as that described in Antibodies, Harlow and Lane (Cold Spring Harbor Press, Cold Spring Harbor, N.Y.). Cross-blocking can be measured in various binding assays such as ELISA, biolayer interferometry, or surface plasmon resonance. Other methods include alanine scanning mutational analysis, peptide blot analysis (Reineke (2004) Methods Mol. Biol. 248: 443-63), peptide cleavage analysis, high-resolution electron microscopy techniques using single particle reconstruction, cryoEM, or tomography, crystallographic studies and NMR analysis. In addition, methods such as epitope excision, epitope extraction and chemical modification of antigens can be employed (Tomer (2000) Prot. Sci. 9: 487-496). Another method that can be used to identify the amino acids within a. polypeptide with which an antibody interacts is hydrogen/deuterium exchange detected by mass spectrometry. In general terms, the hydrogen/deuterium exchange method involves deuterium-labeling the protein of interest, followed by binding the antibody to the deuterium-labeled protein. Next, the protein/antibody complex is transferred to water and exchangeable protons within amino acids that are protected by the antibody complex undergo deuterium-to-hydrogen back-exchange at a slower rate than exchangeable protons within amino acids that are not part of the interface. As a result, amino acids that form part of the protein/antibody interface may retain deuterium and therefore exhibit relatively higher mass compared to amino acids not included in the interface. After dissociation of the antibody, the target protein is subjected to protease cleavage and mass spectrometry analysis, thereby revealing the deuterium-labeled residues which correspond to the specific amino acids with which the antibody interacts. See, e.g., Ehring (1999) Analytical Biochemistry 267: 252-259; Engen and Smith (2001) Anal. Chem. 73: 256A-265A. When the antibody neutralizes Candida, antibody escape mutant variant organisms can be isolated by propagating Candida in vitro or in animal models in the presence of high concentrations of the antibody. Sequence analysis of the Candida gene encoding the antigen targeted by the antibody reveals the mutation(s) conferring antibody escape, indicating residues in the epitope or that affect the structure of the epitope allosterically.

The term “epitope” can refer to a site on an antigen to which B and/or T cells respond. B-cell epitopes can be formed both from contiguous amino acids or noncontiguous amino acids juxtaposed by tertiary folding of a protein. Epitopes formed from contiguous amino acids are typically retained on exposure to denaturing solvents, whereas epitopes formed by tertiary folding are typically lost on treatment with denaturing solvents. An epitope typically includes at least 3, and more usually, at least 5 or 8-10 amino acids in a unique spatial conformation.

Modification-Assisted Profiling (MAP), also known as Antigen Structure-based Antibody Profiling (ASAP) is a method that categorizes large numbers of monoclonal antibodies (mAbs) directed against the same antigen according to the similarities of the binding profile of each antibody to chemically or enzymatically modified antigen surfaces (see US 2004/0101920, herein specifically incorporated by reference in its entirety). Each category can reflect a unique epitope either distinctly different from or partially overlapping with epitope represented by another category. This technology allows rapid filtering of genetically identical antibodies, such that characterization can be focused on genetically distinct antibodies. When applied to hybridoma screening, MAP can facilitate identification of rare hybridoma clones that produce mAbs having the desired characteristics. MAP can be used to sort the antibodies of the disclosure into groups of antibodies binding different epitopes.

The present disclosure includes antibodies that can bind to the same epitope, or a portion of the epitope. Likewise, the present disclosure also includes antibodies that compete for binding to a target or a fragment thereof with any of the specific exemplary antibodies described herein. One can easily determine whether an antibody binds to the same epitope as, or competes for binding with, a reference antibody by using routine methods known in the art. For example, to determine if a test antibody binds to the same epitope as a reference, the reference antibody is allowed to bind to target under saturating conditions. Next, the ability of a test antibody to bind to the target molecule is assessed. If the test antibody is able to bind to the target molecule following saturation binding with the reference antibody, it can be concluded that the test antibody binds to a different epitope than the reference antibody. On the other hand, if the test antibody is not able to bind to the target molecule following saturation binding with the reference antibody, then the test antibody can bind to the same epitope as the epitope bound by the reference antibody.

To determine if an antibody competes for binding with a reference anti-Candida antibody, the above-described binding methodology is performed in two orientations: In a first orientation, the reference antibody is allowed to bind to the Candida antigen under saturating conditions followed by assessment of binding of the test antibody to the Candida antigen. In a second orientation, the test antibody is allowed to bind to the Candida antigen molecule under saturating conditions followed by assessment of binding of the reference antibody to the Candida antigen. If, in both orientations, only the first (saturating) antibody is capable of binding to the Candida antigen, then it is concluded that the test antibody and the reference antibody compete for binding to the Candida antigen. As will be appreciated by a person of ordinary skill in the art, an antibody that competes for binding with a reference antibody may not necessarily bind to the identical epitope as the reference antibody but may sterically block binding of the reference antibody by binding an overlapping or adjacent epitope.

Two antibodies bind to the same or overlapping epitope if each competitively inhibits (blocks) binding of the other to the antigen. That is, a 1-, 5-, 10-, 20- or 100-fold excess of one antibody inhibits binding of the other by at least 50% but preferably 75%, 90% or even 99% as measured in a competitive binding assay (see, e.g., Junghans et al., Cancer Res. 1990 50:1495-1502). Alternatively, two antibodies have the same epitope if essentially all amino acid mutations in the antigen that reduce or eliminate binding of one antibody reduce or eliminate binding of the other. Two antibodies have overlapping epitopes if some amino acid mutations that reduce or eliminate binding of one antibody reduce or eliminate binding of the other.

Additional routine experimentation (e.g., peptide mutation and binding analyses) can then be carried out to confirm whether the observed lack of binding of the test antibody is in fact due to binding to the same epitope as the reference antibody or if steric blocking (or another phenomenon) is responsible for the lack of observed binding. Experiments of this sort can be performed using ELISA, RIA, surface plasmon resonance, flow cytometry or any other quantitative or qualitative antibody-binding assay available in the art. Structural studies with EM or crystallography also can demonstrate whether or not two antibodies that compete for binding recognize the same epitope.

In another aspect, there are provided monoclonal antibodies having clone-paired CDRs from the heavy and light chains as illustrated in Tables 3 and 4, respectively. The monoclonal antibodies can have clone-paired CDRs with at least 70% identity to sequences set forth in Tables 3 and 4. In certain embodiments, the antibody or antibody fragment is about 70%, about 80%, or about 90% identical to the sequences from Tables 3 and 4. Such antibodies can be produced by the clones discussed below in the Examples section using methods described herein.

In another aspect, the antibodies can be characterized by their variable sequence, which include additional “framework” regions. These are provided in Tables 1 and 2 that encode or represent full variable regions. Furthermore, the antibodies sequences can vary from these sequences, optionally using methods discussed in greater detail below. For example, nucleic acid sequences can vary from those set out above in that (a) the variable regions can be segregated away from the constant domains of the light and heavy chains, (b) the nucleic acids can vary from those set out above while not affecting the residues encoded thereby, (c) the nucleic acids can vary from those set out above by a given percentage, e.g., 70%, 75%, 80%, 85%, 90%, 91%, 92%, 93%, 94%, 95%, 96%, 97%, 98% or 99% homology, (d) the nucleic acids can vary from those set out above by virtue of the ability to hybridize under high stringency conditions, as exemplified by low salt and/or high temperature conditions, such as provided by about 0.02 M to about 0.15 M NaCl at temperatures of about 50° C. to about 70° C., (e) the amino acids can vary from those set out above by a given percentage, e.g., 85%, 90%, 91%, 92%, 93%, 94%, 95%, 96%, 97%, 98% or 99% homology, or (f) the amino acids can vary from those set out above by permitting conservative substitutions (discussed below). Each of the foregoing applies to the nucleic acid sequences set forth as Table 1 and the amino acid sequences of Table 2.

Aspects of the disclosure feature antibodies that have a specified percentage identity or similarity to the amino acid or nucleotide sequences of the anti-Candida antibodies or antibody fragments described herein. For example, the antibodies can have 60%, 70%, 75%, 80%, 85%, 90%, 91%, 92%, 93%, 94%, 95%, 96%, 97%, 98%, 99%, or higher identity when compared a specified region or the full length of any one of the anti-Candida antibodies or antibody fragments described herein. When comparing polynucleotide and polypeptide sequences, two sequences are said to be “identical” if the sequence of nucleotides or amino acids in the two sequences is the same when aligned for maximum correspondence, as described below. Comparisons between two sequences are typically performed by comparing the sequences over a comparison window to identify and compare local regions of sequence similarity. A “comparison window” as used herein, refers to a segment of at least about 20 contiguous positions, usually 30 to about 75, 40 to about 50, in which a sequence can be compared to a reference sequence of the same number of contiguous positions after the two sequences are optimally aligned.

Optimal alignment of sequences for comparison can be conducted using the Megalign program in the Lasergene suite of bioinformatics software (DNASTAR, Inc., Madison, Wis.), using default parameters. This program embodies several alignment schemes described in the following references: Dayhoff, M. O. (1978) A model of evolutionary change in proteins—Matrices for detecting distant relationships. In Dayhoff, M. O. (ed.) Atlas of Protein Sequence and Structure, National Biomedical Research Foundation, Washington D.C. Vol. 5, Suppl. 3, pp. 345-358; Hein J. (1990) Unified Approach to Alignment and Phylogeny pp. 626-645 Methods in Enzymology vol. 183, Academic Press, Inc., San Diego, Calif.; Higgins, D. G. and Sharp, P. M. (1989) CABIOS 5:151-153; Myers, E. W. and Muller W. (1988) CABIOS 4:11-17; Robinson, E. D. (1971) Comb. Theor 11:105; Santou, N. Nes, M. (1987) Mol. Biol. Evol. 4:406-425; Sneath, P. H. A. and Sokal, R. R. (1973) Numerical Taxonomy—the Principles and Practice of Numerical Taxonomy, Freeman Press, San Francisco, Calif.; Wilbur, W. J. and Lipman, D. J. (1983) Proc. Natl. Acad., Sci. USA 80:726-730.

Alternatively, optimal alignment of sequences for comparison can be conducted by the local identity algorithm of Smith and Waterman (1981) Add. APL. Math 2:482, by the identity alignment algorithm of Needleman and Wunsch (1970) J. Mol. Biol. 48:443, by the search for similarity methods of Pearson and Lipman (1988) Proc. Natl. Acad. Sci. USA 85: 2444, by computerized implementations of these algorithms (GAP, BESTFIT, BLAST, FASTA, and TFASTA in the Wisconsin Genetics Software Package, Genetics Computer Group (GCG), 575 Science Dr., Madison, Wis.), or by inspection.

One particular example of algorithms that are suitable for determining percent sequence identity and sequence similarity are the BLAST and BLAST 2.0 algorithms, which are described in Altschul et al. (1977) Nucl. Acids Res. 25:3389-3402 and Altschul et al. (1990) J. Mol. Biol. 215:403-410, respectively. BLAST and BLAST 2.0 can be used, for example with the parameters described herein, to determine percent sequence identity for the polynucleotides and polypeptides of the disclosure. Software for performing BLAST analyses is publicly available through the National Center for Biotechnology Information. The rearranged nature of an antibody sequence and the variable length of each gene requires multiple rounds of BLAST searches for a single antibody sequence. Also, manual assembly of different genes is difficult and error-prone. The sequence analysis tool IgBLAST (world-wide-web at ncbi.nlm.nih.gov/igblast/) identifies matches to the germline V, D and J genes, details at rearrangement junctions, the delineation of Ig V domain framework regions and complementarity determining regions. IgBLAST can analyze nucleotide or protein sequences and can process sequences in batches and allows searches against the germline gene databases and other sequence databases simultaneously to obtain the best matching germline V gene.

In one illustrative example, cumulative scores can be calculated using, for nucleotide sequences, the parameters M (reward score for a pair of matching residues; always >0) and N (penalty score for mismatching residues; always <0). Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T and X determine the sensitivity and speed of the alignment. The BLASTN program (for nucleotide sequences) uses as defaults a wordlength (W) of 11, and expectation (E) of 10, and the BLOSUM62 scoring matrix (see Henikoff and Henikoff (1989) Proc. Natl. Acad. Sci. USA 89:10915) alignments, (B) of 50, expectation (E) of 10, M=5, N=−4 and a comparison of both strands.

For amino acid sequences, a scoring matrix can be used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T and X determine the sensitivity and speed of the alignment.

In one approach, the “percentage of sequence identity” is determined by comparing two optimally aligned sequences over a window of comparison of at least 20 positions, wherein the portion of the polynucleotide or polypeptide sequence in the comparison window can comprise additions or deletions (i.e., gaps) of 20 percent or less, usually 5 to 15 percent, or 10 to 12 percent, as compared to the reference sequences (which does not comprise additions or deletions) for optimal alignment of the two sequences. The percentage is calculated by determining the number of positions at which the identical nucleic acid bases or amino acid residues occur in both sequences to yield the number of matched positions, dividing the number of matched positions by the total number of positions in the reference sequence (i.e., the window size) and multiplying the results by 100 to yield the percentage of sequence identity.

A “derivative” of any of the below-described antibodies and their antigen-binding fragments can refer to an antibody or antigen-binding fragment thereof that immunospecifically binds to an antigen but which comprises, one, two, three, four, five or more amino acid substitutions, additions, deletions or modifications relative to a “parental” (or wild-type) molecule. Such amino acid substitutions or additions can introduce naturally occurring (i.e., DNA-encoded) or non-naturally occurring amino acid residues. The term “derivative” encompasses, for example, as variants having altered CH1, hinge, CH2, CH3 or CH4 regions, so as to form, for example antibodies, etc., having variant Fc regions that exhibit enhanced or impaired effector or binding characteristics. The term “derivative” additionally encompasses non-amino acid modifications, for example, amino acids that can be glycosylated (e.g., have altered mannose, 2-N-acetylglucosamine, galactose, fucose, glucose, sialic acid, 5-N-acetylneuraminic acid, 5-glycolneuraminic acid, etc. content), acetylated, pegylated, phosphorylated, amidated, derivatized by known protecting/blocking groups, proteolytic cleavage, linked to a cellular ligand or other protein, etc. In some embodiments, the altered carbohydrate modifications modulate one or more of the following: solubilization of the antibody, facilitation of subcellular transport and secretion of the antibody, promotion of antibody assembly, conformational integrity, and antibody-mediated effector function. In a specific embodiment, the altered carbohydrate modifications enhance antibody mediated effector function relative to the antibody lacking the carbohydrate modification. Carbohydrate modifications that lead to altered antibody mediated effector function are well known in the art (for example, see Shields, R. L. et al. (2002) “Lack Of Fucose On Human IgG N-Linked Oligosaccharide Improves Binding To Human Fcgamma RIII And Antibody-Dependent Cellular Toxicity,” J. Biol. Chem. 277(30): 26733-26740; Davies J. et al. (2001) “Expression Of GnTIII In A Recombinant Anti-CD20 CHO Production Cell Line: Expression Of Antibodies With Altered Glycoforms Leads To An Increase In ADCC Through Higher Affinity For FC Gamma RIII,” Biotechnology & Bioengineering 74(4): 288-294). Methods of altering carbohydrate contents are known to those skilled in the art, see, e.g., Wallick, S. C. et al. (1988) “Glycosylation Of A VH Residue Of A Monoclonal Antibody Against Alpha (1-6) Dextran Increases Its Affinity For Antigen,” J. Exp. Med. 168(3): 1099-1109; Tao, M. H. et al. (1989) “Studies Of Aglycosylated Chimeric Mouse-Human IgG. Role Of Carbohydrate In The Structure And Effector Functions Mediated By The Human IgG Constant Region,” J. Immunol. 143(8): 2595-2601; Routledge, E. G. et al. (1995) “The Effect Of Aglycosylation On The Immunogenicity Of A Humanized Therapeutic CD3 Monoclonal Antibody,” Transplantation 60(8):847-53; Elliott, S. et al. (2003) “Enhancement Of Therapeutic Protein In Vivo Activities Through Glycoengineering,” Nature Biotechnol. 21:414-21; Shields, R. L. et al. (2002) “Lack Of Fucose On Human IgG N-Linked Oligosaccharide Improves Binding To Human Fcgamma RIII And Antibody-Dependent Cellular Toxicity,” J. Biol. Chem. 277(30): 26733-26740).

A derivative antibody or antibody fragment can be generated with an engineered sequence or glycosylation state to confer preferred levels of activity in antibody dependent cellular cytotoxicity (ADCC), antibody-dependent cellular phagocytosis (ADCP), antibody-dependent neutrophil phagocytosis (ADNP), or antibody-dependent complement deposition (ADCD) functions as measured by bead-based or cell-based assays or in vivo studies in animal models.

A derivative antibody or antibody fragment can be modified by chemical modifications using techniques known to those of skill in the art, including, but not limited to, specific chemical cleavage, acetylation, formulation, metabolic synthesis of tunicamycin, etc. In one embodiment, an antibody derivative will possess a similar or identical function as the parental antibody. In another embodiment, an antibody derivative will exhibit an altered activity relative to the parental antibody. For example, a derivative antibody (or fragment thereof) can bind to its epitope more tightly or be more resistant to proteolysis than the parental antibody.

C. Engineering of Antibody Sequences

In various embodiments, one can choose to engineer sequences of the identified antibodies for a variety of reasons, such as improved expression, improved cross-reactivity or diminished off-target binding. Modified antibodies can be made by any technique known to those of skill in the art, including expression through standard molecular biological techniques, or the chemical synthesis of polypeptides. Methods for recombinant expression are addressed elsewhere in this document. The following is a general discussion of relevant goals techniques for antibody engineering.

Hybridomas can be cultured, then cells lysed, and total RNA extracted. Random hexamers can be used with RT to generate cDNA copies of RNA, and then PCR performed using a multiplex mixture of PCR primers expected to amplify all human variable gene sequences. PCR product can be cloned into pGEM-T Easy vector, then sequenced by automated DNA sequencing using standard vector primers. Assay of binding and neutralization can be performed using antibodies collected from hybridoma supernatants and purified by FPLC, using Protein G columns.

An antibody of the present disclosure can be expressed by a vector (also referred to herein as an “expression vector”) containing a DNA segment encoding any single chain antibody described herein. These can include vectors, liposomes, naked DNA, adjuvant-assisted DNA, gene gun, catheters, etc. Vectors include chemical conjugates such as described in WO 93/64701, which has targeting moiety (e.g., a ligand to a cellular surface receptor), and a nucleic acid binding moiety (e.g., polylysine), viral vector (e.g., a DNA or RNA viral vector), fusion proteins such as described in PCT/US 95/02140 (WO 95/22618) which is a fusion protein containing a target moiety (e.g., an antibody specific for a target cell) and a nucleic acid binding moiety (e.g., a protamine), plasmids, phage, etc. The vectors can be chromosomal, non-chromosomal or synthetic.

Vectors can include viral vectors, fusion proteins and chemical conjugates. Retroviral vectors include moloney murine leukemia viruses. DNA viral vectors are preferred. These vectors include pox vectors such as orthopox or avipox vectors, herpesvirus vectors such as a herpes simplex I virus (HSV) vector (see Geller. et al., J. Neurochem, 64:487 (1995); Lim et al., in DNA Cloning: Mammalian Systems, D. Glover, Ed. (Oxford Univ. Press, Oxford England) (1995); Geller et al., Prod Natl. Acad. Sci. USA 90:7603 (1993); Geller et al., Proc. Nat'l Acad. Sci. USA 87: 1149 (1990), Adenovirus Vectors (see LeGal LaSalle et al., Science, 259:988 (1993); Davidson et al., Nat. Genet. 3:219 (1993); Yang et al., Virol. 69:2004 (1995) and Adeno-associated Virus Vectors (see Kaplitt et al., Nat. Genet. 8: 148 (1994).

Pox viral vectors introduce the gene into the cell's cytoplasm. Avipox virus vectors result in only a short-term expression of the nucleic acid. Adenovirus vectors, adeno- associated virus vectors and herpes simplex virus (HSV) vectors are preferred for introducing the nucleic acid into neural cells. The adenovirus vector results in a shorter-term expression (about 2 months) than adeno-associated virus (about 4 months), which in turn is shorter than HSV vectors. The particular vector chosen will depend upon the target cell and the condition being treated. The introduction can be by standard techniques, e.g., infection, transfection, transduction or transformation. Examples of modes of gene transfer include, e.g., naked DNA, CaP04 precipitation, DEAE dextran, electroporation, protoplast fusion, lipofection, cell microinjection, and viral vectors.

These vectors can be used to express large quantities of antibodies that can be used in a variety of ways. For example, to detect the presence of Candida in a sample. The antibody can also be used to try to bind to and disrupt Candida activity.

Recombinant full-length IgG antibodies can be generated by subcloning heavy and light chain Fv DNAs from the cloning vector into an IgG plasmid vector, transfected into 293 (e.g., Freestyle) cells or CHO cells, and antibodies can be collected and purified from the 293 or CHO cell supernatant. Other appropriate host cells systems include bacteria, such as E. coli, insect cells (S2, Sf9, Sf29, High Five), plant cells (e.g., tobacco, with or without engineering for human-like glycans), algae, or in a variety of non-human transgenic contexts, such as mice, rats, goats or cows.

Expression of nucleic acids encoding antibodies, both for the purpose of subsequent antibody purification, and for immunization of a host, can also be practiced according to the invention. Antibody coding sequences can be RNA, such as native RNA or modified RNA. Modified RNA can contain, for example, certain chemical modifications that confer increased stability and low immunogenicity to mRNAs, thereby facilitating expression of therapeutically important proteins. For instance, N1-methyl-pseudouridine (N1mΨ) outperforms several other nucleoside modifications and their combinations in terms of translation capacity. In addition to turning off the immune/eIF2α phosphorylation-dependent inhibition of translation, incorporated N1mΨ nucleotides dramatically alter the dynamics of the translation process by increasing ribosome pausing and density on the mRNA. Increased ribosome loading of modified mRNAs renders them more permissive for initiation by favoring either ribosome recycling on the same mRNA or de novo ribosome recruitment. Such modifications could be used to enhance antibody expression in vivo following inoculation with RNA. The RNA, whether native or modified, can be delivered as naked RNA or in a delivery vehicle, such as a lipid nanoparticle.

Alternatively, DNA encoding the antibody can be employed for the same purposes. The DNA is included in an expression cassette comprising a promoter active in the host cell for which it is designed. The expression cassette is advantageously included in a replicable vector, such as a conventional plasmid or minivector. Vectors include viral vectors, such as poxviruses, adenoviruses, herpesviruses, adeno-associated viruses, and lentiviruses can be used. Replicons encoding antibody genes such as alphavirus replicons based on VEE virus or Sindbis virus are also can also be utilized. Delivery of such vectors can be performed by needle through intramuscular, subcutaneous, or intradermal routes, or by transcutaneous electroporation when in vivo expression is desired.

The rapid availability of antibody produced in the same host cell and cell culture process as the final cGMP manufacturing process can reduce the duration of process development programs. Lonza has developed a generic method using pooled transfectants grown in CDACF medium, for the rapid production of small quantities (up to 50 g) of antibodies in CHO cells. Although slightly slower than a true transient system, the advantages include a higher product concentration and use of the same host and process as the production cell line. Example of growth and productivity of GS-CHO pools, expressing a model antibody, in a disposable bioreactor: in a disposable bag bioreactor culture (5 L working volume) operated in fed-batch mode, a harvest antibody concentration of 2 g/L was achieved within 9 weeks of transfection.

Antibody molecules will comprise fragments (such as F(ab′), F(ab′)₂) that are produced, for example, by the proteolytic cleavage of the mAbs, or single-chain immunoglobulins producible, for example, via recombinant means. F(ab′) antibody derivatives are monovalent, while F(ab′)₂ antibody derivatives are bivalent. In one embodiment, such fragments can be combined with one another, or with other antibody fragments or receptor ligands to form “chimeric” binding molecules. Significantly, such chimeric molecules can contain substituents capable of binding to different epitopes of the same molecule.

In related embodiments, the antibody is a derivative of the disclosed antibodies, e.g., an antibody comprising the CDR sequences identical to those in the disclosed antibodies (e.g., a chimeric, or CDR-grafted antibody). Alternatively, one can make modifications, such as introducing conservative changes into an antibody molecule. In making such changes, the hydropathic index of amino acids can be considered. The importance of the hydropathic amino acid index in conferring interactive biologic function on a protein is generally understood in the art (Kyte and Doolittle, 1982). It is accepted that the relative hydropathic character of the amino acid contributes to the secondary structure of the resultant protein, which in turn defines the interaction of the protein with other molecules, for example, enzymes, substrates, receptors, DNA, antibodies, antigens, and the like.

The substitution of like amino acids can be made effectively on the basis of hydrophilicity. U.S. Pat. No. 4,554,101, incorporated herein by reference, states that the greatest local average hydrophilicity of a protein, as governed by the hydrophilicity of its adjacent amino acids, correlates with a biological property of the protein. As detailed in U.S. Pat. No. 4,554,101, the following hydrophilicity values have been assigned to amino acid residues: basic amino acids: arginine (+3.0), lysine (+3.0), and histidine (−0.5); acidic amino acids: aspartate (+3.0±1), glutamate (+3.0±1), asparagine (+0.2), and glutamine (+0.2); hydrophilic, nonionic amino acids: serine (+0.3), asparagine (+0.2), glutamine (+0.2), and threonine (−0.4), sulfur containing amino acids: cysteine (−1.0) and methionine (−1.3); hydrophobic, nonaromatic amino acids: valine (−1.5), leucine (−1.8), isoleucine (−1.8), proline (−0.5±1), alanine (−0.5), and glycine (0); hydrophobic, aromatic amino acids: tryptophan (−3.4), phenylalanine (−2.5), and tyrosine (−2.3).

For example, an amino acid can be substituted for another having a similar hydrophilicity and produce a biologically or immunologically modified protein. In such changes, the substitution of amino acids whose hydrophilicity values are within ±2 is preferred, those that are within ±1 are particularly preferred, and those within ±0.5 are even more particularly preferred.

As outlined above, amino acid substitutions generally are based on the relative similarity of the amino acid side-chain substituents, for example, their hydrophobicity, hydrophilicity, charge, size, and the like. Exemplary substitutions that take into consideration the various foregoing characteristics are well known to those of skill in the art and include: arginine and lysine; glutamate and aspartate; serine and threonine; glutamine and asparagine; and valine, leucine and isoleucine.

The present disclosure also is directed to isotype modification. By modifying the Fc region to have a different isotype, different functionalities can be achieved. For example, changing to IgG₁ can increase antibody dependent cell cytotoxicity, switching to class A can improve tissue distribution, and switching to class M can improve valency.

Alternatively or additionally, it can be useful to combine amino acid modifications with one or more further amino acid modifications that alter C1q binding and/or the complement dependent cytotoxicity (CDC) function of the Fc region of an IL-23p19 binding molecule. The binding polypeptide of particular interest can be one that binds to C1q and displays complement dependent cytotoxicity. Polypeptides with pre-existing C1q binding activity, optionally further having the ability to mediate CDC can be modified such that one or both of these activities are enhanced. Amino acid modifications that alter C1q and/or modify its complement dependent cytotoxicity function are described, for example, in WO/0042072, which is hereby incorporated by reference.

One can design an Fc region of an antibody with altered effector function, e.g., by modifying C1q binding and/or FcγR binding and thereby changing CDC activity and/or ADCC activity. “Effector functions” are responsible for activating or diminishing a biological activity (e.g., in a subject). Examples of effector functions include, but are not limited to: C1q binding; complement dependent cytotoxicity (CDC); Fc receptor binding; antibody-dependent cell-mediated cytotoxicity (ADCC); phagocytosis; down regulation of cell surface receptors (e.g., B cell receptor; BCR), etc. Such effector functions can require the Fc region to be combined with a binding domain (e.g., an antibody variable domain) and can be assessed using various assays (e.g., Fc binding assays, ADCC assays, CDC assays, etc.).

For example, one can generate a variant Fc region of an antibody with improved C1q binding and improved FcγRIII binding (e.g., having both improved ADCC activity and improved CDC activity). Alternatively, if it is desired that effector function be reduced or ablated, a variant Fc region can be engineered with reduced CDC activity and/or reduced ADCC activity. In other embodiments, only one of these activities can be increased, and, optionally, also the other activity reduced (e.g., to generate an Fc region variant with improved ADCC activity, but reduced CDC activity and vice versa).

FcRn binding. Fc mutations can also be introduced and engineered to alter their interaction with the neonatal Fc receptor (FcRn) and improve their pharmacokinetic properties. A collection of human Fc variants with improved binding to the FcRn have been described (Shields et al., (2001). High resolution mapping of the binding site on human IgG1 for FcγRI, FcγRII, FcγRIII, and FcRn and design of IgG1 variants with improved binding to the FcγR, (J. Biol. Chem. 276:6591-6604). A number of methods are known that can result in increased half-life (Kuo and Aveson, (2011)), including amino acid modifications can be generated through techniques including alanine scanning mutagenesis, random mutagenesis and screening to assess the binding to the neonatal Fc receptor (FcRn) and/or the in vivo behavior. Computational strategies followed by mutagenesis can also be used to select one of amino acid mutations to mutate.

The present disclosure therefore provides a variant of an antigen binding protein with optimized binding to FcRn. In a particular embodiment, the said variant of an antigen binding protein comprises at least one amino acid modification in the Fc region of said antigen binding protein, wherein said modification is selected from the group consisting of 226, 227, 228, 230, 231, 233, 234, 239, 241, 243, 246, 250, 252, 256, 259, 264, 265, 267, 269, 270, 276, 284, 285, 288, 289, 290, 291, 292, 294, 297, 298, 299, 301, 302, 303, 305, 307, 308, 309, 311, 315, 317, 320, 322, 325, 327, 330, 332, 334, 335, 338, 340, 342, 343, 345, 347, 350, 352, 354, 355, 356, 359, 360, 361, 362, 369, 370, 371, 375, 378, 380, 382, 384, 385, 386, 387, 389, 390, 392, 393, 394, 395, 396, 397, 398, 399, 400, 401 403, 404, 408, 411, 412, 414, 415, 416, 418, 419, 420, 421, 422, 424, 426, 428, 433, 434, 438, 439, 440, 443, 444, 445, 446 and 447 of the Fc region as compared to said parent polypeptide, wherein the numbering of the amino acids in the Fc region is that of the EU index in Rabat. In a further aspect of the disclosure the modifications are M252Y/S254T/T256E.

Additionally, various publications describe methods for obtaining physiologically active molecules whose half-lives are modified, see for example Kontermann (2009) either by introducing an FcRn-binding polypeptide into the molecules or by fusing the molecules with antibodies whose FcRn-binding affinities are preserved but affinities for other Fc receptors have been greatly reduced or fusing with FcRn binding domains of antibodies.

Derivatized antibodies can be used to alter the half-lives (e.g., serum half-lives) of parental antibodies in a mammal, particularly a human. Such alterations can result in a half-life of greater than 15 days, preferably greater than 20 days, greater than 25 days, greater than 30 days, greater than 35 days, greater than 40 days, greater than 45 days, greater than 2 months, greater than 3 months, greater than 4 months, or greater than 5 months. The increased half-lives of the antibodies of the present disclosure or fragments thereof in a mammal, preferably a human, results in a higher serum titer of said antibodies or antibody fragments in the mammal, and thus reduces the frequency of the administration of said antibodies or antibody fragments and/or reduces the concentration of said antibodies or antibody fragments to be administered. Antibodies or fragments thereof having increased in vivo half-lives can be generated by techniques known to those of skill in the art. For example, antibodies or fragments thereof with increased in vivo half-lives can be generated by modifying (e.g., substituting, deleting or adding) amino acid residues identified as involved in the interaction between the Fc domain and the FcRn receptor.

Beltramello et al. (2010) previously reported the modification of neutralizing mAbs, due to their tendency to enhance dengue virus infection, by generating in which leucine residues at positions 1.3 and 1.2 of CH2 domain (according to the IMGT unique numbering for C-domain) were substituted with alanine residues. This modification, also known as “LALA” mutation, abolishes antibody binding to FcγRI, FcγRII and FcγRIIIa, as described by Hessell et al. (2007). The variant and unmodified recombinant mAbs were compared for their capacity to neutralize and enhance infection by the four dengue virus serotypes. LALA variants retained the same neutralizing activity as unmodified mAb but were completely devoid of enhancing activity. LALA mutations of this nature can also be used with the presently disclosed antibodies.

Altered Glycosylation. A particular embodiment of the present disclosure is an isolated monoclonal antibody, or antigen binding fragment thereof, containing a substantially homogeneous glycan without sialic acid, galactose, or fucose. In embodiments, the monoclonal antibody comprises a heavy chain variable region and a light chain variable region, both of which can be attached to heavy chain or light chain constant regions respectively. The aforementioned substantially homogeneous glycan can be covalently attached to the heavy chain constant region.

Another embodiment of the present disclosure comprises a mAb with a new Fc glycosylation pattern. The isolated monoclonal antibody, or antigen binding fragment thereof, is present in a substantially homogenous composition represented by the GNGN or G1/G2 glycoform. Fc glycosylation plays a significant role in anti-viral and anti-cancer properties of therapeutic mAbs. The is in line with a recent study that shows increased anti-lentivirus cell-mediated viral inhibition of a fucose free anti-HIV mAb in vitro.

The isolated monoclonal antibody, or antigen binding fragment thereof, comprising a substantially homogenous composition represented by the GNGN or G1/G2 glycoform exhibits increased binding affinity for Fc gamma RI and Fc gamma RIII compared to the same antibody without the substantially homogeneous GNGN glycoform and with G0, G1F, G2F, GNF, GNGNF or GNGNFX containing glycoforms. In one embodiment of the present disclosure, the antibody dissociates from Fc gamma RI with a Kd of 1×10⁻⁸M or less and from Fc gamma RIII with a Kd of 1×10⁻⁷ M or less.

Glycosylation of an Fc region is typically either N-linked or O-linked. N-linked refers to the attachment of the carbohydrate moiety to the side chain of an asparagine residue, O-linked glycosylation refers to the attachment of one of the sugars N-acetylgalactosamine, galactose, or xylose to a hydroxyamino acid, most commonly serine or threonine, although 5-hydroxyproline or 5-hydroxylysine can also be used. The recognition sequences for enzymatic attachment of the carbohydrate moiety to the asparagine side chain peptide sequences are asparagine-X-serine and asparagine-X-threonine, where X is any amino acid except proline. Thus, the presence of either of these peptide sequences in a polypeptide can create a glycosylation site.

The glycosylation pattern can be altered, for example, by deleting one or more glycosylation site(s) found in the polypeptide, and/or adding one or more glycosylation site(s) that are not present in the polypeptide. Addition of glycosylation sites to the Fc region of an antibody is conveniently accomplished by altering the amino acid sequence such that it contains one or more of the above-described tripeptide sequences (for N-linked glycosylation sites). An exemplary glycosylation variant has an amino acid substitution of residue Asn 297 of the heavy chain. The alteration can also be made by the addition of, or substitution by, one or more serine or threonine residues to the sequence of the original polypeptide (for O-linked glycosylation sites). Additionally, a change of Asn 297 to Ala can remove one of the glycosylation sites.

In certain embodiments, the antibody is expressed in cells that express beta (1,4)-N-acetylglucosaminyltransferase III (GnT III), such that GnT III adds GlcNAc to the IL-23p19 antibody. Methods for producing antibodies in such a fashion are provided in WO/9954342, WO/03011878, patent publication 20030003097A1, and Umana et al., Nature Biotechnology, 17:176-180, February 1999. Cell lines can be altered to enhance or reduce or eliminate certain post-translational modifications, such as glycosylation, using genome editing technology such as Clustered Regularly Interspaced Short Palindromic Repeats (CRISPR). For example, CRISPR technology can be used to eliminate genes encoding glycosylating enzymes in 293 or CHO cells used to express recombinant monoclonal antibodies.

Elimination of monoclonal antibody protein sequence liabilities. Antibody variable gene sequences obtained from human B cells can be engineered to enhance their manufacturability and safety. Protein sequence liabilities can be identified by searching for sequence motifs associated with sites containing:

1) Unpaired Cys residues,

2) N-linked glycosylation,

3) Asn deamidation,

4) Asp isomerization,

5) SYE truncation,

6) Met oxidation,

7) Trp oxidation,

8) N-terminal glutamate,

9) Integrin binding,

10) CD11c/CD18 binding, or

11) Fragmentation

Such motifs can be eliminated by altering the synthetic gene for the cDNA encoding recombinant antibodies.

Protein engineering efforts in the field of development of therapeutic antibodies clearly reveal that certain sequences or residues are associated with solubility differences (Fernandez-Escamilla et al., Nature Biotech., 22 (10), 1302-1306, 2004; Chennamsetty et al., PNAS, 106 (29), 11937-11942, 2009; Voynov et al., Biocon. Chem., 21 (2), 385-392, 2010) Evidence from solubility-altering mutations in the literature indicate that some hydrophilic residues such as aspartic acid, glutamic acid, and serine contribute significantly more favorably to protein solubility than other hydrophilic residues, such as asparagine, glutamine, threonine, lysine, and arginine.

Stability. Antibodies can be engineered for enhanced biophysical properties. One can use elevated temperature to unfold antibodies to determine relative stability, using average apparent melting temperatures. Differential Scanning Calorimetry (DSC) measures the heat capacity, Cₚ, of a molecule (the heat required to warm it, per degree) as a function of temperature. One can use DSC to study the thermal stability of antibodies. DSC data for mAbs is particularly interesting because it sometimes resolves the unfolding of individual domains within the mAb structure, producing up to three peaks in the thermogram (from unfolding of the Fab, C_{H}2, and C_{H}3 domains). Typically unfolding of the Fab domain produces the strongest peak. The DSC profiles and relative stability of the Fc portion show characteristic differences for the human IgG₁, IgG₂, IgG₃, and IgG₄ subclasses (Garber and Demarest, Biochem. Biophys. Res. Commun. 355, 751-757, 2007). One also can determine average apparent melting temperature using circular dichroism (CD), performed with a CD spectrometer. Far-UV CD spectra will be measured for antibodies in the range of 200 to 260 nm at increments of 0.5 nm. The final spectra can be determined as averages of 20 accumulations. Residue ellipticity values can be calculated after background subtraction. Thermal unfolding of antibodies (0.1 mg/mL) can be monitored at 235 nm from 25-95° C. and a heating rate of 1° C./min. One can use dynamic light scattering (DLS) to assess for propensity for aggregation. DLS is used to characterize size of various particles including proteins. If the system is not disperse in size, the mean effective diameter of the particles can be determined. This measurement depends on the size of the particle core, the size of surface structures, and particle concentration. Since DLS essentially measures fluctuations in scattered light intensity due to particles, the diffusion coefficient of the particles can be determined. DLS software in commercial DLA instruments displays the particle population at different diameters. Stability studies can be done conveniently using DLS. DLS measurements of a sample can show whether the particles aggregate over time or with temperature variation by determining whether the hydrodynamic radius of the particle increases. If particles aggregate, one can see a larger population of particles with a larger radius. Stability depending on temperature can be analyzed by controlling the temperature in situ. Capillary electrophoresis (CE) techniques include proven methodologies for determining features of antibody stability. One can use an iCE approach to resolve antibody protein charge variants due to deamidation, C-terminal lysines, sialylation, oxidation, glycosylation, and any other change to the protein that can result in a change in pI of the protein. Each of the expressed antibody proteins can be evaluated by high throughput, free solution isoelectric focusing (IEF) in a capillary column (cIEF), using a Protein Simple Maurice instrument. Whole-column UV absorption detection can be performed every 30 seconds for real time monitoring of molecules focusing at the isoelectric points (pIs). This approach combines the high resolution of traditional gel IEF with the advantages of quantitation and automation found in column-based separations while eliminating the need for a mobilization step. The technique yields reproducible, quantitative analysis of identity, purity, and heterogeneity profiles for the expressed antibodies. The results identify charge heterogeneity and molecular sizing on the antibodies, with both absorbance and native fluorescence detection modes and with sensitivity of detection down to 0.7 μg/mL.

Solubility. One can determine the intrinsic solubility score of antibody sequences. The intrinsic solubility scores can be calculated using CamSol Intrinsic (Sormanni et al., J Mol Biol 427, 478-490, 2015). The amino acid sequences for residues 95-102 (Kabat numbering) in HCDR3 of each antibody fragment such as a scFv can be evaluated via the online program to calculate the solubility scores. One also can determine solubility using laboratory techniques. Various techniques exist, including addition of lyophilized protein to a solution until the solution becomes saturated and the solubility limit is reached, or concentration by ultrafiltration in a microconcentrator with a suitable molecular weight cut-off. The most straightforward method is induction of amorphous precipitation, which measures protein solubility using a method involving protein precipitation using ammonium sulfate (Trevino et al., J Mol Biol, 366: 449-460, 2007). Ammonium sulfate precipitation gives quick and accurate information on relative solubility values. Ammonium sulfate precipitation produces precipitated solutions with well-defined aqueous and solid phases and requires relatively small amounts of protein. Solubility measurements performed using induction of amorphous precipitation by ammonium sulfate also can be done easily at different pH values. Protein solubility is highly pH dependent, and pH is considered the most important extrinsic factor that affects solubility.

Autoreactivity. Generally, it is thought that autoreactive clones should be eliminated during ontogeny by negative selection, however it has become clear that many human naturally-occurring antibodies with autoreactive properties persist in adult mature repertoires, and the autoreactivity can enhance the anti-pathogen function of many antibodies to pathogens. It has been noted that HCDR3 loops in antibodies during early B cell development are often rich in positive charge and exhibit autoreactive patterns (Wardemann et al., Science 301, 1374-1377, 2003). One can test a given antibody for autoreactivity by assessing the level of binding to human origin cells in microscopy (using adherent HeLa or HEp-2 epithelial cells) and flow cytometric cell surface staining (using suspension Jurkat T cells and 293S human embryonic kidney cells). Autoreactivity also can be surveyed using assessment of binding to tissues in tissue arrays.

Preferred residues (“Human Likeness”). B cell repertoire deep sequencing of human B cells from blood donors is being performed on a wide scale in many recent studies. Sequence information about a significant portion of the human antibody repertoire facilitates statistical assessment of antibody sequence features common in healthy humans. With knowledge about the antibody sequence features in a human recombined antibody variable gene reference database, the position specific degree of “Human Likeness” (HL) of an antibody sequence can be estimated. HL has been shown to be useful for the development of antibodies in clinical use, like therapeutic antibodies or antibodies as vaccines. The goal is to increase the human likeness of antibodies to reduce adverse effects and anti-antibody immune responses that will lead to significantly decreased efficacy of the antibody drug or can induce serious health implications. One can assess antibody characteristics of the combined antibody repertoire of three healthy human blood donors of about 400 million sequences in total and created a new “relative Human Likeness” (rHL) score that focuses on the hypervariable region of the antibody. The rHL score allows one to easily distinguish between human (positive score) and non-human sequences (negative score). Antibodies can be engineered to eliminate residues that are not common in human repertoires.

D. Single Chain Antibodies

A single chain variable fragment (scFv) is a fusion of the variable regions of the heavy and light chains of immunoglobulins, linked together with a short (usually serine, glycine) linker. This chimeric molecule retains the specificity of the original immunoglobulin, despite removal of the constant regions and the introduction of a linker peptide. This modification usually leaves the specificity unaltered. These molecules were created historically to facilitate phage display where it is highly convenient to express the antigen binding domain as a single peptide. Alternatively, scFv can be created directly from subcloned heavy and light chains derived from a hybridoma or B cell. Single chain variable fragments lack the constant Fc region found in complete antibody molecules, and thus, the common binding sites (e.g., protein A/G) used to purify antibodies. These fragments can often be purified/immobilized using Protein L since Protein L interacts with the variable region of kappa light chains.

Flexible linkers generally are comprised of helix- and turn-promoting amino acid residues such as alanine, serine and glycine. However, other residues can function as well. Tang et al. (1996) used phage display as a means of rapidly selecting tailored linkers for single-chain antibodies (scFvs) from protein linker libraries. A random linker library was constructed in which the genes for the heavy and light chain variable domains were linked by a segment encoding an 18-amino acid polypeptide of variable composition. The scFv repertoire (approx. 5×10⁶ different members) was displayed on filamentous phage and subjected to affinity selection with hapten. The population of selected variants exhibited significant increases in binding activity but retained considerable sequence diversity. Screening 1054 individual variants subsequently yielded a catalytically active scFv that was produced efficiently in soluble form. Sequence analysis revealed a conserved proline in the linker two residues after the V_{H} C terminus and an abundance of arginines and prolines at other positions as the only common features of the selected tethers.

The recombinant antibodies of the present disclosure can also involve sequences or moieties that permit dimerization or multimerization of the receptors. Such sequences include those derived from IgA, which permit formation of multimers in conjunction with the J-chain. Another multimerization domain is the Gal4 dimerization domain. In other embodiments, the chains can be modified with agents such as biotin/avidin, which permit the combination of two antibodies.

In a separate embodiment, a single-chain antibody can be created by joining receptor light and heavy chains using a non-peptide linker or chemical unit. Generally, the light and heavy chains will be produced in distinct cells, purified, and subsequently linked together in an appropriate fashion (i.e., the N-terminus of the heavy chain being attached to the C-terminus of the light chain via an appropriate chemical bridge).

Cross-linking reagents are used to form molecular bridges that tie functional groups of two different molecules, e.g., a stabilizing and coagulating agent. However, dimers or multimers of the same analog or heteromeric complexes comprised of different analogs can be created. To link two different compounds in a step-wise manner, hetero-bifunctional cross-linkers can be used that eliminate unwanted homopolymer formation.

An exemplary hetero-bifunctional cross-linker contains two reactive groups: one reacting with primary amine group (e.g., N-hydroxy succinimide) and the other reacting with a thiol group (e.g., pyridyl disulfide, maleimides, halogens, etc.). Through the primary amine reactive group, the cross-linker can react with the lysine residue(s) of one protein (e.g., the selected antibody or fragment) and through the thiol reactive group, the cross-linker, already tied up to the first protein, reacts with the cysteine residue (free sulfhydryl group) of the other protein (e.g., the selective agent).

In embodiments, a cross-linker having reasonable stability in blood can be employed. Numerous types of disulfide-bond containing linkers are known that can be successfully employed to conjugate targeting and therapeutic/preventative agents. Linkers that contain a disulfide bond that is sterically hindered may prove to give greater stability in vivo, preventing release of the targeting peptide prior to reaching the site of action. These linkers are thus one group of linking agents.

Another cross-linking reagent is SMPT, which is a bifunctional cross-linker containing a disulfide bond that is “sterically hindered” by an adjacent benzene ring and methyl groups. It is believed that steric hindrance of the disulfide bond serves a function of protecting the bond from attack by thiolate anions such as glutathione which can be present in tissues and blood, and thereby help in preventing decoupling of the conjugate prior to the delivery of the attached agent to the target site.

The SMPT cross-linking reagent, as with many other known cross-linking reagents, lends the ability to cross-link functional groups such as the SH of cysteine or primary amines (e.g., the epsilon amino group of lysine). Another type of cross-linker includes the hetero-bifunctional photoreactive phenylazides containing a cleavable disulfide bond such as sulfosuccinimidyl-2-(p-azido salicylamide) ethyl-1,3′-dithiopropionate. The N-hydroxy-succinimidyl group reacts with primary amino groups and the phenylazide (upon photolysis) reacts non-selectively with any amino acid residue.

In addition to hindered cross-linkers, non-hindered linkers also can be employed in accordance herewith. Other useful cross-linkers, not considered to contain or generate a protected disulfide, include SATA, SPDP and 2-iminothiolane (Wawrzynczak & Thorpe, 1987). The use of such cross-linkers is well understood in the art. Another embodiment involves the use of flexible linkers.

U.S. Pat. No. 4,680,338 describes bifunctional linkers useful for producing conjugates of ligands with amine-containing polymers and/or proteins, especially for forming antibody conjugates with chelators, drugs, enzymes, detectable labels and the like. U.S. Pat. Nos. 5,141,648 and 5,563,250 disclose cleavable conjugates containing a labile bond that is cleavable under a variety of mild conditions. This linker is particularly useful in that the agent of interest can be bonded directly to the linker, with cleavage resulting in release of the active agent. Particular uses include adding a free amino or free sulfhydryl group to a protein, such as an antibody, or a drug.

U.S. Pat. No. 5,856,456 provides peptide linkers for use in connecting polypeptide constituents to make fusion proteins, e.g., single chain antibodies. The linker is up to about 50 amino acids in length, contains at least one occurrence of a charged amino acid (preferably arginine or lysine) followed by a proline, and is characterized by greater stability and reduced aggregation. U.S. Pat. No. 5,880,270 discloses aminooxy-containing linkers useful in a variety of immunodiagnostic and separative techniques.

E. Multispecific Antibodies

In certain embodiments, antibodies of the present disclosure are bispecific or multispecific. Bispecific antibodies are antibodies that have binding specificities for at least two different epitopes. Exemplary bispecific antibodies can bind to two different epitopes of a single antigen. Other such antibodies can combine a first antigen binding site with a binding site for a second antigen. Alternatively, an anti-pathogen arm can be combined with an arm that binds to a triggering molecule on a leukocyte, such as a T-cell receptor molecule (e.g., CD3), or Fc receptors for IgG (FcγR), such as FcγRI (CD64), FcγRII (CD32) and Fc gamma RIII (CD16), so as to focus and localize cellular defense mechanisms to the infected cell. Bispecific antibodies can also be used to localize cytotoxic agents to infected cells. These antibodies possess a pathogen-binding arm and an arm that binds the cytotoxic agent (e.g., saporin, anti-interferon-α, vinca alkaloid, ricin A chain, methotrexate or radioactive isotope hapten). Bispecific antibodies can be prepared as full-length antibodies or antibody fragments (e.g., F(ab′)₂ bispecific antibodies). WO 96/16673 describes a bispecific anti-ErbB2/anti-Fc gamma RIII antibody and U.S. Pat. No. 5,837,234 discloses a bispecific anti-ErbB2/anti-Fc gamma RI antibody. A bispecific anti-ErbB2/Fc alpha antibody is shown in WO98/02463. U.S. Pat. No. 5,821,337 teaches a bispecific anti-ErbB2/anti-CD3 antibody.

Methods for making bispecific antibodies are known in the art. Traditional production of full-length bispecific antibodies is based on the co-expression of two immunoglobulin heavy chain-light chain pairs, where the two chains have different specificities (Millstein et al., Nature, 305:537-539 (1983)). Because of the random assortment of immunoglobulin heavy and light chains, these hybridomas (quadromas) can produce a mixture of ten different antibody molecules, of which only one has the correct bispecific structure. Purification of the correct molecule, which is usually done by affinity chromatography steps, is rather cumbersome, and the product yields are low. Similar procedures are disclosed in WO 93/08829, and in Traunecker et al., EMBO J., 10:3655-3659 (1991).

According to a different approach, antibody variable regions with the desired binding specificities (antibody-antigen combining sites) are fused to immunoglobulin constant domain sequences. Preferably, the fusion is with an Ig heavy chain constant domain, comprising at least part of the hinge, C_{H2}, and C_{H3} regions. It is preferred to have the first heavy-chain constant region (C_{H1}) containing the site necessary for light chain bonding, present in at least one of the fusions. DNAs encoding the immunoglobulin heavy chain fusions and, if desired, the immunoglobulin light chain, are inserted into separate expression vectors, and are co-transfected into a suitable host cell. This provides for greater flexibility in adjusting the mutual proportions of the three polypeptide fragments in embodiments when unequal ratios of the three polypeptide chains used in the construction provide the optimum yield of the desired bispecific antibody. Coding sequences can be inserted for two or all three polypeptide chains into a single expression vector when the expression of at least two polypeptide chains in equal ratios results in high yields or when the ratios have no significant effect on the yield of the desired chain combination.

In a particular embodiment of this approach, the bispecific antibodies are composed of a hybrid immunoglobulin heavy chain with a first binding specificity in one arm, and a hybrid immunoglobulin heavy chain-light chain pair (providing a second binding specificity) in the other arm. It was found that this asymmetric structure facilitates the separation of the desired bispecific compound from unwanted immunoglobulin chain combinations, as the presence of an immunoglobulin light chain in only one half of the bispecific molecule provides for a facile way of separation. This approach is disclosed in WO 94/04690. For further details of generating bispecific antibodies see, for example, Suresh et al., Methods in Enzymology, 121:210 (1986).

According to another approach described in U.S. Pat. No. 5,731,168, the interface between a pair of antibody molecules can be engineered to maximize the percentage of heterodimers that are recovered from recombinant cell culture. The preferred interface comprises at least a part of the C_{H3} domain. In this method, one or more small amino acid side chains from the interface of the first antibody molecule are replaced with larger side chains (e.g., tyrosine or tryptophan). Compensatory “cavities” of identical or similar size to the large side chain(s) are created on the interface of the second antibody molecule by replacing large amino acid side chains with smaller ones (e.g., alanine or threonine). This provides a mechanism for increasing the yield of the heterodimer over other unwanted end-products such as homodimers.

Bispecific antibodies include cross-linked or “heteroconjugate” antibodies. For example, one of the antibodies in the heteroconjugate can be coupled to avidin, the other to biotin. Such antibodies have, for example, been proposed to target immune system cells to unwanted cells (U.S. Pat. No. 4,676,980), and for treatment of HIV infection (WO 91/00360, WO 92/200373, and EP 03089). Heteroconjugate antibodies can be made using any convenient cross-linking methods. Suitable cross-linking agents are well known in the art, and are disclosed in U.S. Pat. No. 4,676,980, along with a number of cross-linking techniques.

Techniques for generating bispecific antibodies from antibody fragments have also been described in the literature. For example, bispecific antibodies can be prepared using chemical linkage. Brennan et al., Science, 229: 81 (1985) describe a procedure wherein intact antibodies are proteolytically cleaved to generate F(ab′)₂ fragments. These fragments are reduced in the presence of the dithiol complexing agent, sodium arsenite, to stabilize vicinal dithiols and prevent intermolecular disulfide formation. The Fab′ fragments generated are then converted to thionitrobenzoate (TNB) derivatives. One of the Fab′-TNB derivatives is then reconverted to the Fab′-thiol by reduction with mercaptoethylamine and is mixed with an equimolar amount of the other Fab′-TNB derivative to form the bispecific antibody. The bispecific antibodies produced can be used as agents for the selective immobilization of enzymes.

Techniques exist that facilitate the direct recovery of Fab′-SH fragments from E. coli, which can be chemically coupled to form bispecific antibodies. Shalaby et al., J. Exp. Med., 175: 217-225 (1992) describe the production of a humanized bispecific antibody F(ab′)₂ molecule. Each Fab′ fragment was separately secreted from E. coli and subjected to directed chemical coupling in vitro to form the bispecific antibody. The bispecific antibody thus formed was able to bind to cells overexpressing the ErbB2 receptor and normal human T cells, as well as trigger the lytic activity of human cytotoxic lymphocytes against human breast tumor targets.

Various techniques for making and isolating bispecific antibody fragments directly from recombinant cell culture have also been described (Merchant et al., Nat. Biotechnol. 16, 677-681 (1998). doi:10.1038/nbt0798-677pmid:9661204). For example, bispecific antibodies have been produced using leucine zippers (Kostelny et al., J. Immunol., 148(5):1547-1553, 1992). The leucine zipper peptides from the Fos and Jun proteins were linked to the Fab′ portions of two different antibodies by gene fusion. The antibody homodimers were reduced at the hinge region to form monomers and then re-oxidized to form the antibody heterodimers. This method can also be utilized for the production of antibody homodimers. The “diabody” technology described by Hollinger et al., Proc. Natl. Acad. Sci. USA, 90:6444-6448 (1993) has provided an alternative mechanism for making bispecific antibody fragments. The fragments comprise a V_{H} connected to a V_{L} by a linker that is too short to allow pairing between the two domains on the same chain. Accordingly, the V_{H} and V_{L} domains of one fragment are forced to pair with the complementary V_{L} and V_{H} domains of another fragment, thereby forming two antigen-binding sites. Another strategy for making bispecific antibody fragments by the use of single-chain Fv (sFv) dimers has also been reported. See Gruber et al., J. Immunol., 152:5368 (1994).

In a particular embodiment, a bispecific or multispecific antibody can be formed as a DOCK-AND-LOCK™ (DNL™) complex (see, e.g., U.S. Pat. Nos. 7,521,056; 7,527,787; 7,534,866; 7,550,143 and 7,666,400, the Examples section of each of which is incorporated herein by reference.) Generally, the technique takes advantage of the specific and high-affinity binding interactions that occur between a dimerization and docking domain (DDD) sequence of the regulatory (R) subunits of cAMP-dependent protein kinase (PKA) and an anchor domain (AD) sequence derived from any of a variety of AKAP proteins (Baillie et al., FEBS Letters. 2005; 579: 3264; Wong and Scott, Nat. Rev. Mol. Cell Biol. 2004; 5: 959). The DDD and AD peptides can be attached to any protein, peptide or other molecule. Because the DDD sequences spontaneously dimerize and bind to the AD sequence, the technique allows the formation of complexes between any selected molecules that can be attached to DDD or AD sequences.

Antibodies with more than two valencies can also be produced. For example, trispecific antibodies can be prepared (Tutt et al., J. Immunol. 147: 60, 1991; Xu et al., Science, 358(6359):85-90, 2017). A multivalent antibody can be internalized (and/or catabolized) faster than a bivalent antibody by a cell expressing an antigen to which the antibodies bind. The antibodies of the present disclosure can be multivalent antibodies with three or more antigen binding sites (e.g., tetravalent antibodies), which can be readily produced by recombinant expression of nucleic acid encoding the polypeptide chains of the antibody. The multivalent antibody can comprise a dimerization domain and three or more antigen binding sites. The preferred dimerization domain comprises (or consists of) an Fc region or a hinge region. In this scenario, the antibody will comprise an Fc region and three or more antigen binding sites amino-terminal to the Fc region. The preferred multivalent antibody herein comprises (or consists of) three to about eight, but preferably four, antigen binding sites. The multivalent antibody comprises at least one polypeptide chain (and preferably two polypeptide chains), wherein the polypeptide chain(s) comprise two or more variable regions. For instance, the polypeptide chain(s) can comprise VD1-(X1)ₙ-VD2-(X2)ₐ-Fc, wherein VD1 is a first variable region, VD2 is a second variable region, Fc is one polypeptide chain of an Fc region, X1 and X2 represent an amino acid or polypeptide, and n is 0 or 1. For instance, the polypeptide chain(s) can comprise: VH-CH1-flexible linker-VH-CH1-Fc region chain; or VH-CH1-VH-CH1-Fc region chain. The multivalent antibody herein preferably further comprises at least two (and preferably four) light chain variable region polypeptides. The multivalent antibody herein can, for instance, comprise from about two to about eight light chain variable region polypeptides. The light chain variable region polypeptides comprise a light chain variable region and, optionally, further comprise a C_{L} domain.

Charge modifications are particularly useful in the context of a multi specific antibody, where amino acid substitutions in Fab molecules result in reducing the mispairing of light chains with non-matching heavy chains (Bence-Jones-type side products), which can occur in the production of Fab-based bi-/multispecific antigen binding molecules with a VH/VL exchange in one (or more, in case of molecules comprising more than two antigen-binding Fab molecules) of their binding arms (see also PCT publication no. WO 2015/150447, particularly the examples therein, incorporated herein by reference in its entirety).

Accordingly, in particular embodiments, an antibody comprised in the therapeutic agent comprises
(a) a first Fab molecule which specifically binds to a first antigen
(b) a second Fab molecule which specifically binds to a second antigen, and wherein the variable domains VL and VH of the Fab light chain and the Fab heavy chain are replaced by each other,
wherein the first antigen is an activating T cell antigen and the second antigen is a target cell antigen, or the first antigen is a target cell antigen and the second antigen is an activating T cell antigen; and
wherein
i) in the constant domain CL of the first Fab molecule under a) the amino acid at position 124 is substituted by a positively charged amino acid (numbering according to Kabat), and wherein in the constant domain CH1 of the first Fab molecule under a) the amino acid at position 147 or the amino acid at position 213 is substituted by a negatively charged amino acid (numbering according to Kabat EU index); or
ii) in the constant domain CL of the second Fab molecule under b) the amino acid at position 124 is substituted by a positively charged amino acid (numbering according to Kabat), and wherein in the constant domain CH1 of the second Fab molecule under b) the amino acid at position 147 or the amino acid at position 213 is substituted by a negatively charged amino acid (numbering according to Kabat EU index).
   In certain embodiments, the antibody does not comprise both modifications mentioned under i) and ii). In embodiments, the constant domains CL and CH1 of the second Fab molecule are not replaced by each other (i.e., remain unexchanged).

In another embodiment of the antibody, in the constant domain CL of the first Fab molecule under a) the amino acid at position 124 is substituted independently by lysine (K), arginine (R) or histidine (H) (numbering according to Kabat) (in one preferred embodiment independently by lysine (K) or arginine (R)), and in the constant domain CH1 of the first Fab molecule under a) the amino acid at position 147 or the amino acid at position 213 is substituted independently by glutamic acid (E), or aspartic acid (D) (numbering according to Kabat EU index).

In a further embodiment, in the constant domain CL of the first Fab molecule under a) the amino acid at position 124 is substituted independently by lysine (K), arginine (R) or histidine (H) (numbering according to Kabat), and in the constant domain CH1 of the first Fab molecule under a) the amino acid at position 147 is substituted independently by glutamic acid (E), or aspartic acid (D) (numbering according to Kabat EU index).

In a particular embodiment, in the constant domain CL of the first Fab molecule under a) the amino acid at position 124 is substituted independently by lysine (K), arginine (R) or histidine (H) (numbering according to Kabat) (in one preferred embodiment independently by lysine (K) or arginine (R)) and the amino acid at position 123 is substituted independently by lysine (K), arginine (R) or histidine (H) (numbering according to Kabat) (in one preferred embodiment independently by lysine (K) or arginine (R)), and in the constant domain CH1 of the first Fab molecule under a) the amino acid at position 147 is substituted independently by glutamic acid (E), or aspartic acid (D) (numbering according to Kabat EU index) and the amino acid at position 213 is substituted independently by glutamic acid (E), or aspartic acid (D) (numbering according to Kabat EU index).

In a more particular embodiment, in the constant domain CL of the first Fab molecule under a) the amino acid at position 124 is substituted by lysine (K) (numbering according to Kabat) and the amino acid at position 123 is substituted by lysine (K) or arginine (R) (numbering according to Kabat), and in the constant domain CH1 of the first Fab molecule under a) the amino acid at position 147 is substituted by glutamic acid (E) (numbering according to Kabat EU index) and the amino acid at position 213 is substituted by glutamic acid (E) (numbering according to Kabat EU index).

In an even more particular embodiment, in the constant domain CL of the first Fab molecule under a) the amino acid at position 124 is substituted by lysine (K) (numbering according to Kabat) and the amino acid at position 123 is substituted by arginine (R) (numbering according to Kabat), and in the constant domain CH1 of the first Fab molecule under a) the amino acid at position 147 is substituted by glutamic acid (E) (numbering according to Kabat EU index) and the amino acid at position 213 is substituted by glutamic acid (E) (numbering according to Kabat EU index).

F. Chimeric Antigen Receptors

Artificial T cell receptors (also known as chimeric T cell receptors, chimeric immunoreceptors, chimeric antigen receptors (CARs)) are engineered receptors, which graft an arbitrary specificity onto an immune effector cell. Typically, these receptors are used to graft the specificity of a monoclonal antibody onto a T cell, with transfer of their coding sequence facilitated by retroviral vectors. In this way, a large number of target-specific T cells can be generated for adoptive cell transfer. Phase I clinical studies of this approach show efficacy.

The most common form of these molecules are fusions of single-chain variable fragments (scFv) derived from monoclonal antibodies, fused to CD3-zeta transmembrane and endodomain. Such molecules result in the transmission of a zeta signal in response to recognition by the scFv of its target. An example of such a construct is 14g2a-Zeta, which is a fusion of a scFv derived from hybridoma 14g2a (which recognizes disialoganglioside GD2). When T cells express this molecule (usually achieved by oncoretroviral vector transduction), they recognize and kill target cells that express GD2 (e.g., neuroblastoma cells). To target malignant B cells, investigators have redirected the specificity of T cells using a chimeric immunoreceptor specific for the B-lineage molecule, CD19.

The variable portions of an immunoglobulin heavy and light chain are fused by a flexible linker to form a scFv. This scFv is preceded by a signal peptide to direct the nascent protein to the endoplasmic reticulum and subsequent surface expression (this is cleaved). A flexible spacer allows to the scFv to orient in different directions to allow antigen binding. The transmembrane domain is a typical hydrophobic alpha helix usually derived from the original molecule of the signaling endodomain which protrudes into the cell and transmits the desired signal.

Type I proteins are in fact two protein domains linked by a transmembrane alpha helix in between. The cell membrane lipid bilayer, through which the transmembrane domain passes, acts to isolate the inside portion (endodomain) from the external portion (ectodomain). It is not so surprising that attaching an ectodomain from one protein to an endodomain of another protein results in a molecule that combines the recognition of the former to the signal of the latter.

Ectodomain. A signal peptide directs the nascent protein into the endoplasmic reticulum. This is essential if the receptor is to be glycosylated and anchored in the cell membrane. Any eukaryotic signal peptide sequence usually works fine. Generally, the signal peptide natively attached to the amino-terminal most component is used (e.g., in a scFv with orientation light chain-linker-heavy chain, the native signal of the light-chain is used

The antigen recognition domain is usually an scFv. There are however many alternatives. An antigen recognition domain from native T-cell receptor (TCR) alpha and beta single chains have been described, as have simple ectodomains (e.g., CD4 ectodomain to recognize HIV infected cells) and more exotic recognition components such as a linked cytokine (which leads to recognition of cells bearing the cytokine receptor). In fact, almost anything that binds a given target with high affinity can be used as an antigen recognition region.

A spacer region links the antigen binding domain to the transmembrane domain. It should be flexible enough to allow the antigen binding domain to orient in different directions to facilitate antigen recognition. The simplest form is the hinge region from IgG1. Alternatives include the CH₂CH₃ region of immunoglobulin and portions of CD3. For most scFv based constructs, the IgG1 hinge suffices. However, the best spacer often has to be determined empirically.

Transmembrane domain. The transmembrane domain is a hydrophobic alpha helix that spans the membrane. Generally, the transmembrane domain from the most membrane proximal component of the endodomain is used. Interestingly, using the CD3-zeta transmembrane domain can result in incorporation of the artificial TCR into the native TCR a factor that is dependent on the presence of the native CD3-zeta transmembrane charged aspartic acid residue. Different transmembrane domains result in different receptor stability. The CD28 transmembrane domain results in a brightly expressed, stable receptor.

Endodomain. This is the “business-end” of the receptor. After antigen recognition, receptors cluster and a signal is transmitted to the cell. The most commonly used endodomain component is CD3-zeta which contains 3 ITAMs. This transmits an activation signal to the T cell after antigen is bound. CD3-zeta may not provide a fully competent activation signal and additional co-stimulatory signaling is needed.

“First-generation” CARs typically had the intracellular domain from the CD3 chain, which is the primary transmitter of signals from endogenous TCRs. “Second-generation” CARs add intracellular signaling domains from various costimulatory protein receptors (e.g., CD28, 41BB, ICOS) to the cytoplasmic tail of the CAR to provide additional signals to the T cell. Preclinical studies have indicated that the second generation of CAR designs improves the antitumor activity of T cells. More recent, “third-generation” CARs combine multiple signaling domains, such as CD3z-CD28-41BB or CD3z-CD28-OX40, to further augment potency.

G. ADCs

Antibody Drug Conjugates or ADCs are a new class of highly potent biopharmaceutical drugs designed as a targeted therapy for the treatment of people with infectious disease. ADCs are complex molecules composed of an antibody (a whole mAb or an antibody fragment such as a single-chain variable fragment, or scFv) linked, via a stable chemical linker with labile bonds, to a biological active cytotoxic/anti-pathogen payload or drug. Antibody Drug Conjugates are examples of bioconjugates and immunoconjugates.

By combining the unique targeting capabilities of monoclonal antibodies with the cancer-killing ability of cytotoxic drugs, antibody-drug conjugates allow sensitive discrimination between healthy and diseased tissue. This means that, in contrast to traditional systemic approaches, antibody-drug conjugates target and attack the infected cell so that healthy cells are less severely affected.

In the development ADC-based anti-tumor therapies, an anticancer drug (e.g., a cell toxin or cytotoxin) is coupled to an antibody that specifically targets a certain cell marker (e.g., a protein that, ideally, is only to be found in or on infected cells). Antibodies track these proteins down in the body and attach themselves to the surface of cancer cells. The biochemical reaction between the antibody and the target protein (antigen) triggers a signal in the tumor cell, which then absorbs or internalizes the antibody together with the cytotoxin. After the ADC is internalized, the cytotoxic drug is released and kills the cell or impairs pathogen's replication. Due to this targeting, ideally the drug has lower side effects and gives a wider therapeutic window than other agents.

A stable link between the antibody and cytotoxic/anti-pathogen agent is a crucial aspect of an ADC. Linkers are based on chemical motifs including disulfides, hydrazones or peptides (cleavable), or thioethers (noncleavable) and control the distribution and delivery of the cytotoxic agent to the target cell. Cleavable and noncleavable types of linkers have been proven to be safe in preclinical and clinical trials. Brentuximab vedotin includes an enzyme-sensitive cleavable linker that delivers the potent and highly toxic antimicrotubule agent Monomethyl auristatin E or MMAE, a synthetic antineoplastic agent, to human specific CD30-positive malignant cells. Because of its high toxicity MMAE, which inhibits cell division by blocking the polymerization of tubulin, cannot be used as a single-agent chemotherapeutic drug. However, the combination of MMAE linked to an anti-CD30 monoclonal antibody (cAC10, a cell membrane protein of the tumor necrosis factor or TNF receptor) proved to be stable in extracellular fluid, cleavable by cathepsin and safe for therapy. Trastuzumab emtansine, the other approved ADC, is a combination of the microtubule-formation inhibitor mertansine (DM-1), a derivative of the Maytansine, and antibody trastuzumab (Herceptin®/Genentech/Roche) attached by a stable, non-cleavable linker.

The availability of better and more stable linkers has changed the function of the chemical bond. The type of linker, cleavable or noncleavable, lends specific properties to the cytotoxic (anti-cancer) drug. For example, a non-cleavable linker keeps the drug within the cell. As a result, the entire antibody, linker and cytotoxic agent enter the targeted cancer cell where the antibody is degraded to the level of an amino acid. The resulting complex—amino acid, linker and cytotoxic agent—now becomes the active drug. In contrast, cleavable linkers are catalyzed by enzymes in the host cell where it releases the cytotoxic agent.

Another type of cleavable linker, currently in development, adds an extra molecule between the cytotoxic/anti-pathogen drug and the cleavage site. This linker technology allows researchers to create ADCs with more flexibility without worrying about changing cleavage kinetics. Researchers are also developing a new method of peptide cleavage based on Edman degradation, a method of sequencing amino acids in a peptide. Future direction in the development of ADCs also include the development of site-specific conjugation (TDCs) to further improve stability and therapeutic index and a emitting immunoconjugates and antibody-conjugated nanoparticles.

H. BiTES

Bi-specific T-cell engagers (BiTEs) are a class of artificial bispecific monoclonal antibodies that are investigated for the use as anti-cancer drugs. They direct a host's immune system, more specifically the T cells' cytotoxic activity, against infected cells. BiTE is a registered trademark of Micromet AG.

BiTEs are fusion proteins comprising two single-chain variable fragments (scFvs) of different antibodies, or amino acid sequences from four different genes, on a single peptide chain of about 55 kilodaltons. One of the scFvs binds to T cells via the CD3 receptor, and the other to an infected cell via a specific molecule.

Like other bispecific antibodies, and unlike ordinary monoclonal antibodies, BiTEs form a link between T cells and target cells. This causes T cells to exert cytotoxic/anti-pathogen activity on infected cells by producing proteins like perforin and granzymes, independently of the presence of MHC I or co-stimulatory molecules. These proteins enter infected cells and initiate the cell's apoptosis. This action mimics physiological processes observed during T cell attacks against infected cells.

I. Intrabodies

In a particular embodiment, the antibody is a recombinant antibody that is suitable for action inside of a cell—such antibodies are known as “intrabodies.” These antibodies can interfere with target function by a variety of mechanism, such as by altering intracellular protein trafficking, interfering with enzymatic function, and blocking protein-protein or protein-DNA interactions. In many ways, their structures mimic or parallel those of single chain and single domain antibodies, discussed above. Indeed, single-transcript/single-chain is an important feature that permits intracellular expression in a target cell, and also makes protein transit across cell membranes more feasible. However, additional features are required.

The two major issues impacting the implementation of intrabody therapeutic are delivery, including cell/tissue targeting, and stability. With respect to delivery, a variety of approaches have been employed, such as tissue-directed delivery, use of cell-type specific promoters, viral-based delivery and use of cell-permeability/membrane translocating peptides. With respect to the stability, the approach is generally to either screen by brute force, including methods that involve phage display and can include sequence maturation or development of consensus sequences, or more directed modifications such as insertion stabilizing sequences (e.g., Fc regions, chaperone protein sequences, leucine zippers) and disulfide replacement/modification.

An additional feature that intrabodies can require is a signal for intracellular targeting. Vectors that can target intrabodies (or other proteins) to subcellular regions such as the cytoplasm, nucleus, mitochondria and ER have been designed and are commercially available (Invitrogen Corp.; Persic et al., 1997).

By virtue of their ability to enter cells, intrabodies have additional uses that other types of antibodies may not achieve. In the case of the present antibodies, the ability to interact with the MUC1 cytoplasmic domain in a living cell can interfere with functions associated with the MUC1 CD, such as signaling functions (binding to other molecules) or oligomer formation. In particular, such antibodies can be used to inhibit MUC1 dimer formation.

J. Purification

In certain embodiments, the antibodies of the present disclosure can be purified. The term “purified,” as used herein, can refer to a composition, isolatable from other components, wherein the protein is purified to any degree relative to its naturally-obtainable state. A purified protein therefore also refers to a protein, free from the environment in which it may naturally occur. Where the term “substantially purified” is used, this designation can refer to a composition in which the protein or peptide forms the major component of the composition, such as constituting about 50%, about 60%, about 70%, about 80%, about 90%, about 95% or more of the proteins in the composition.

Protein purification techniques are well known to those of skill in the art. These techniques involve, at one level, the crude fractionation of the cellular milieu to polypeptide and non-polypeptide fractions. Having separated the polypeptide from other proteins, the polypeptide of interest can be further purified using chromatographic and electrophoretic techniques to achieve partial or complete purification (or purification to homogeneity). Analytical methods particularly suited to the preparation of a pure peptide are ion-exchange chromatography, exclusion chromatography; polyacrylamide gel electrophoresis; isoelectric focusing. Other methods for protein purification include, precipitation with ammonium sulfate, PEG, antibodies and the like or by heat denaturation, followed by centrifugation; gel filtration, reverse phase, hydroxylapatite and affinity chromatography; and combinations of such and other techniques.

In purifying an antibody of the present disclosure, it can be desirable to express the polypeptide in a prokaryotic or eukaryotic expression system and extract the protein using denaturing conditions. The polypeptide can be purified from other cellular components using an affinity column, which binds to a tagged portion of the polypeptide. As is generally known in the art, it is believed that the order of conducting the various purification steps can be changed, or that certain steps can be omitted, and still result in a suitable method for the preparation of a substantially purified protein or peptide.

Commonly, complete antibodies are fractionated utilizing agents (i.e., protein A) that bind the Fc portion of the antibody. Alternatively, antigens can be used to simultaneously purify and select appropriate antibodies. Such methods often utilize the selection agent bound to a support, such as a column, filter or bead. The antibodies are bound to a support, contaminants removed (e.g., washed away), and the antibodies released by applying conditions (salt, heat, etc.).

Various methods for quantifying the degree of purification of the protein or peptide will be known to those of skill in the art in light of the present disclosure. These include, for example, determining the specific activity of an active fraction, or assessing the amount of polypeptides within a fraction by SDS/PAGE analysis. Another method for assessing the purity of a fraction is to calculate the specific activity of the fraction, to compare it to the specific activity of the initial extract, and to thus calculate the degree of purity. The actual units used to represent the amount of activity will, of course, be dependent upon the particular assay technique chosen to follow the purification and whether or not the expressed protein or peptide exhibits a detectable activity.

It is known that the migration of a polypeptide can vary, sometimes significantly, with different conditions of SDS/PAGE (Capaldi et al., 1977). It will therefore be appreciated that under differing electrophoresis conditions, the apparent molecular weights of purified or partially purified expression products can vary.

### III. ACTIVE/PASSIVE IMMUNIZATION AND TREATMENT/PREVENTION OF CANDIDA INFECTION

A. Formulation

The present disclosure provides pharmaceutical compositions comprising anti-Candida antibodies and antigens for generating the same. Such compositions comprise a prophylactically or therapeutically effective amount of an antibody or a fragment thereof, or a peptide immunogen, and a pharmaceutically acceptable carrier. As used herein, the term “pharmaceutically acceptable carrier” can include any and all solvents, dispersion media, coatings, antibacterial and antifungal agents, isotonic and absorption delaying agents, and the like, compatible with pharmaceutical administration. In a specific embodiment, the term “pharmaceutically acceptable” can mean approved by a regulatory agency of the Federal or a state government or listed in the U.S. Pharmacopeia or other generally recognized pharmacopeia for use in animals, and more particularly in humans. The term “carrier” can refer to a diluent, excipient, or vehicle with which the therapeutic is administered. Suitable carriers are described in the most recent edition of Remington's Pharmaceutical Sciences, a standard reference text in the field, which is incorporated herein by reference. Such pharmaceutical carriers can be sterile liquids, such as water and oils, including those of petroleum, animal, vegetable or synthetic origin, such as peanut oil, soybean oil, mineral oil, sesame oil and the like. Water is a particular carrier when the pharmaceutical composition is administered intravenously. Saline solutions and aqueous dextrose and glycerol solutions can also be employed as liquid carriers, particularly for injectable solutions. Other suitable pharmaceutical excipients include, but are not limited to, starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like.

The composition, if desired, can also contain minor amounts of wetting or emulsifying agents, or pH buffering agents. These compositions can take the form of solutions, suspensions, emulsion, tablets, pills, capsules, powders, sustained-release formulations and the like. Oral formulations can include standard carriers such as pharmaceutical grades of mannitol, lactose, starch, magnesium stearate, sodium saccharine, cellulose, magnesium carbonate, etc. Examples of suitable pharmaceutical agents are described in “Remington's Pharmaceutical Sciences.” Such compositions will contain a prophylactically or therapeutically effective amount of the antibody or fragment thereof, preferably in purified form, together with a suitable amount of carrier so as to provide the form for proper administration to the patient. The formulation should suit the mode of administration, which can be oral, intravenous, intraarterial, intrabuccal, intranasal, nebulized, bronchial inhalation, intra-rectal, vaginal, topical or delivered by mechanical ventilation. A pharmaceutical composition of the invention is formulated to be compatible with its intended route of administration.

B. Administration

Examples of routes of administration include parenteral, e.g., intravenous, intradermal, subcutaneous, oral (e.g., inhalation), transdermal (i.e., topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid (EDTA); buffers such as acetates, citrates or phosphates, and agents for the adjustment of tonicity such as sodium chloride or dextrose. The pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use can include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In embodiments, the composition is sterile and is fluid to the extent that easy syringeability exists. It can be stable under the conditions of manufacture and storage and can be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as manitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, methods of preparation are vacuum drying and freeze-drying that yields a powder of the active ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser which contains a suitable propellant, e.g., a gas such as carbon dioxide, or a nebulizer.

Systemic administration can also be by transmucosal or transdermal means.

For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The compounds can also be prepared in the form of suppositories (e.g., with conventional suppository bases such as cocoa butter and other glycerides) or retention enemas for rectal delivery.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of individuals.

Active vaccines are also envisioned where antibodies like those disclosed are produced in vivo in a subject at risk of Candida infection. Such vaccines can be formulated for parenteral administration, e.g., formulated for injection via the intradermal, intravenous, intramuscular, subcutaneous, or even intraperitoneal routes. Administration by intradermal and intramuscular routes can be utilized. The vaccine could alternatively be administered by a topical route directly to the mucosa, for example by nasal drops, inhalation, by nebulizer, or via intrarectal or vaginal delivery. Pharmaceutically-acceptable salts, include the acid salts and those which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, 2-ethyl amino ethanol, histidine, procaine, and the like.

Passive transfer of antibodies, known as artificially acquired passive immunity, generally will involve the use of intravenous or intramuscular injections. The forms of antibody can be human or animal blood plasma or serum, as pooled human immunoglobulin for intravenous (IVIG) or intramuscular (IG) use, as high-titer human IVIG or IG from immunized or from donors recovering from disease, and as monoclonal antibodies (MAb). Such immunity generally lasts for only a short period of time, and there is also a risk for hypersensitivity reactions, and serum sickness, especially from gamma globulin of non-human origin. However, passive immunity provides immediate protection. The antibodies will be formulated in a carrier suitable for injection, i.e., sterile and syringeable.

Generally, the ingredients of compositions of the disclosure are supplied either separately or mixed together in unit dosage form, for example, as a dry lyophilized powder or water-free concentrate in a hermetically sealed container such as an ampoule or sachette indicating the quantity of active agent. Where the composition is to be administered by infusion, it can be dispensed with an infusion bottle containing sterile pharmaceutical grade water or saline. Where the composition is administered by injection, an ampoule of sterile water for injection or saline can be provided so that the ingredients can be mixed prior to administration.

The compositions of the disclosure can be formulated as neutral or salt forms. Pharmaceutically acceptable salts include those formed with anions such as those derived from hydrochloric, phosphoric, acetic, oxalic, tartaric acids, etc., and those formed with cations such as those derived from sodium, potassium, ammonium, calcium, ferric hydroxides, isopropylamine, triethylamine, 2-ethylamino ethanol, histidine, procaine, etc.

C. MSC Delivery Approach

Mesenchymal stem cells (MSC) are unique multipotent progenitor cells that are presently being exploited as gene therapy vectors for a variety of conditions, including cancer and autoimmune diseases. Although MSC are predominantly known for anti-inflammatory properties during allogeneic MSC transplant, there is evidence that MSC can actually promote adaptive immunity under certain settings. MSC have been identified in a wide variety of tissues, including bone marrow, adipose tissue, placenta, and umbilical cord blood. Adipose tissue is one of the richest known sources of MSC.

MSC have been successfully transplanted into allogeneic hosts in a variety of clinical and pre-clinical settings. These donor MSC often promote immunotolerance, including the inhibition of graft-versus-host disease (GvHD) that can develop after cell or tissue transplantation from a major histocompatibility complex (MHC)-mismatched donor. The diminished GvHD symptoms after MSC transfer has been due to direct MSC inhibition of T and B cell proliferation, resting natural killer cell cytotoxicity, and dendritic cell (DC) maturation. At least one study has reported generation of antibodies against transplanted allogeneic MSC. Nevertheless, the ability to prevent GvHD also suggests that MSC expressing foreign antigen might have an advantage over other cell types (i.e., DC) during a cellular vaccination in selectively inducing immune responses to only the foreign antigen(s) expressed by MSC and not specifically the donor MSC.

MSC have been studied as a delivery vehicle for anti-cancer therapeutics due to their innate tendency to home to tumor microenvironments. MSC also have been used to promote apoptosis of tumorigenic cells through the expression of IFNα or IFNγ. Additionally, MSC recently have been explored for the prevention and inhibition of tumorigenesis and metastasis. Other studies have indicated that immortalized MSC can become tumorigenic, and thus must be carefully studied to determine if they are indeed safe for use. Transplanted primary non-immortalized MSC persist only for a few days at most in vivo.

Vaccines often are efficient and cost-effective means of preventing infectious disease. Vaccines have demonstrated transformative potential in eradicating one devastating disease, smallpox, while offering the ability to control other diseases, including diphtheria, polio, and measles, that formerly caused widespread morbidity and mortality. Traditional vaccine approaches have, however, thus far failed to provide protection against HIV, tuberculosis, malaria and many other diseases, including dengue, herpes and even the common cold. The reasons why traditional vaccine approaches have not been successful for these diseases are complex and varied. For example, HIV integrates functional proviral genomes into the DNA of host cells, thereby establishing latency or persistence. Once latency/persistence is established, HIV has not been able to be eradicated, even with highly active antiretroviral therapy.

Newer alternative immunization approaches include both DNA and cellular vaccines. DNA vaccines involve the transfection of cells at the tissue site of vaccination with an antigen-encoding plasmid that allows local cells (i.e., myocytes) to produce the vaccine antigen in situ. Cellular vaccines use the direct transfer of pre-pulsed or transfected host antigen presenting cells (e.g., dendritic cells, DC) expressing or presenting the vaccine antigen. The advantage of these approaches is that vaccine antigens are produced in vivo and are readily available for immunological processing. Despite numerous reports of successful pre-clinical testing, both such approaches have hit stumbling blocks. DNA vaccination studies in humans show poor efficacy, which has been linked to innate differences between mice and humans. DC vaccination strategies have shown limited clinical success for therapeutic cancer vaccinations and have high production costs due to necessary individual tailoring.

Here, the inventors envision the use of immunoprotective primary mesenchymal stems cells (IP-MSC), which episomally express antibodies specifically target a Candida, as well as methods of preparing and using the IP-MSC. The IP-MSC are transfected with one or more episomal vectors encoding antigen-binding polypeptides (e.g., full antibodies, single chain variable antibodies fragments (ScFV), Fab or F(ab′)₂ antibody fragments, diabodies, tribodies, and the like). Optionally, the IP-MSC can further express one or more other immunomodulating polypeptides, e.g., a cytokine such as an interleukin (e.g., IL-2, IL-4, IL-6, IL-7, IL-9, and IL-12), an interferon (e.g., IFNα, IFNβ, or IFNω), and the like, which can enhance the effectiveness of the antigen-binding polypeptides to neutralize the fungus. Each immunoreactive polypeptide comprises an amino acid sequence of an antigen-binding region from or of a neutralizing antibody (e.g., a native antibody from an exposed subject) specific for an antigen produced by the fungus. Each antigen-binding region peptide is arranged and oriented to specifically bind to and neutralize the pathogen or toxin.

In some embodiments the IP-MSC express, e.g., 1, 2, 3, 4, 5, or 6 immunoreactive polypeptides, or up to about 10, 20, 30, 40, 50, 60, 70, 80, 90 or 100 immunoreactive polypeptides, which specifically target the pathogen. For example, each immunoreactive polypeptide can specifically target and bind to a protein or fragment thereof from a pathogenic organism.

The IP-MSC are useful for generating passive immunity against or treating an infection by the pathogen. The IP-MSC can be provided in a pharmaceutically acceptable carrier (e.g., a buffer, such as phosphate buffered saline, or any other buffered material suitable for sustaining viable transfected primary MSC) for use as a pharmaceutical composition for treating or preventing an infectious disease caused by the pathogen. In some embodiments, the IP-MSC comprise bone-marrow derived MSC, while in some other embodiments, the IP-MSC comprise adipose MSC cells, placental MSC cells, or umbilical cord blood MSC cells.

The IP-MSC described herein are particularly useful for temporary passive protection against fungi, at least in part, because primary MSC are hypo-immunogenic cells that generally are not targeted by the immune system. Thus, the IP-MSC are tolerated by the treated subject, allowing the cells to survive for a sufficient time for immunoreactive polypeptides to be expressed, produced, and released to bind to and inhibit Candida to which the subject has been or may be exposed. In addition, primary MSC generally have a limited lifetime in the body, thus can ameliorate undesirable long-term side effects of treatment with the MSC (e.g., carcinogenicity), which can be an issue with immortalized MSC.

The inventors employ a multicopy, non-infective, non-integrative, circular episome is used to express protective completely human single chain antibody fragments, full length IgGs, or other immunoreactive polypeptides against multiple (even hundreds) bacterial, viral, fungal, or parasite proteins or protein toxoids simultaneously. In some embodiments, the episome is based on components derived from Epstein-Barr virus (EBV) nuclear antigen 1 expression cassette (EBNA1) and the OriP origin of replication. These preferably are the only components of EBV that are used, so that no viruses are replicated or assembled. This system results in stable extra-chromosomal persistence and long-term ectopic gene expression in mesencymal stem cells. In the methods described herein, ScFVs or other immunoreactive polypeptides are effectively expressed in and secreted from MSC in protective amounts. This technology is described in detail in U.S. Pat. No. 9,101,597, which is incorporated herein by reference. The ability of EBV-based episomes to introduce and maintain very large human genomic DNA fragments (>300 kb) in human cells is another significant advantage of the methods described herein. This feature permits cloning of dozens of expression elements in a vector capable of replicating in bacteria, amenable to large scale purification, transfection into hMSC, and replication as an episomal plasmid. Targeted expression levels for the immunoreactive polypeptides (e.g., ScFVs) are about 10 pg/cell/day for each immunoreactive polypeptide, preferably expression levels of 5 pg/cell/day. An infusion with about 1×10¹¹ MSC with a productivity rate of 10 pg/cell/day for each immunoreactive polypeptide generates about 1 gram of soluble polypeptide per day, equivalent to a 15 mg/mL level in the circulation of a 75 Kg adult, which is a suitable therapeutic dosage level. Promoters and other regulatory elements are used to drive the expression of each type of immunomodulatory molecule.

Several reports in the literature point to a non-classical pattern of expression from well characterized promoters in MSC. The human cytomegalovirus major immediate early gene promoter (CMV-MIE) is one of the strongest promoters known, and a major element in the generation of multi-gram per liter recombinant protein drug producing stable mammalian cell lines. The CMV-MIE is however, relatively poorly transcribed in MSC. In contrast, EF1A, UBC, and CAGG promoters have demonstrated high levels of expression in MSC without obvious signs or promoter silencing. The episomal vectors utilized in the methods described herein can include any such promoters. Expression vectors without antibiotic selection markers also are provided for expansion of plasmids in E. coli. The replicative nature of the episomal plasmid precludes its linearization with a restriction endonuclease that disrupts the antibiotic resistance gene's open reading frame. Thus, it is conceivable that genetic rearrangements would result in expression of an antibiotic resistance gene, that can give rise to undesirable antibiotic resistance-mediated side effects in humans in selected cases. This scenario can be averted by substituting antibiotic resistance genes with metabolic selectable markers for growth and propagation of plasmids in E. coli strains, if needed or desired.

Regulatory elements in the vector are utilized to accommodate desired secreted levels and serum levels of each immunomodulatory molecule of interest. Expression of full-length antibodies, ScFV, or other immunoreactive polypeptides benefit from strong promoters (e.g., CMV, EF1A, CAGG, etc.) to achieve therapeutic serum levels within less than one day after administration of MSCs. Other immunomodulatory molecules, such as cytokines, are often expressed and secreted at low levels, and transiently by MSC. To accommodate required flexibility in disparate levels and timing of expression such genes are driven from low basal promoters (i.e., TK), or through controlled induction from a Tet on/off promoter. The Tet promoter system benefits from the use of innocuous antibiotic analogs such as anhydrotetracycline, which activates the Tet promoter at concentrations 2 logs lower than with tetracycline, does not result in dysregulation of intestinal flora, does not result in resistance to polyketide antibiotics, and does not exhibit antibiotic activity Anhydrotetracycline is fully soluble in water, and can be administered in drinking rations to potentiate activation of selected genes in transfected MSCs. The potential toxicity of anhydrotetracycline, the first breakdown product of tetracycline in the human body, can be circumvented by administration of other analogs, such doxycycline, an FDA-approved tetracycline analog that also activates the Tet on/off promoter system. This system preferentially is employed in the design of a failsafe “kill switch” by tightly regulating inducible expression of a potent pro-apoptotic gene (e.g., Bax) to initiate targeted apoptosis of transfected MSCs in the event of untoward side effects or when the desired therapeutic endpoint has been achieved. Recent advances in the Tet-on system have resulted in much enhanced repression of promoter leakiness and responsiveness to Dox at concentrations up to 100-fold lower than in the original Tet system (Tet-On Advanced™, Tet-On 3G™). Drug selectable markers are not used to maintain vector stability in transfected MSC: EBV-based vectors, which are known to replicate and be retained in daughter cells at a rate of 90-92% per cell cycle.

Because episomes do not produce replicating viruses, and the cells in which they are expressed do not produce MHC molecules in any significant amounts, episomes do not result in vector-derived immunity that would prevent a subsequent use of the platform in an individual. This can be confirmed by designing a sensitive assay to detect immune responses (antibody ELISA and T-cell based assays) to components derived from Epstein-Barr virus (EBV) nuclear antigen 1 expression cassette, and to the MSC background (HLA typing). Genetic studies are performed to investigate rates of EBV integration into the host cell chromosome (FISH, Southern blot, qPCR), and to measure the transient replicative nature of the vector. It has been reported that EBV vectors retain about 90 to 92% replication per cell cycle in the absence of a selectable marker. A decreasing replication rate contributes to the clearance of the vector from the host system. Compartmentalization of injected MSC is assessed in non-human primates (NHP) by tracking fluorescently labeled cells preloaded with cell membrane permeable dyes (green CMFDA, orange CMTMR) that upon esterification will no longer cross the lipid bilayer and become highly fluorescent. Such measurements are performed on freshly prepared tissue sections (lymph nodes, liver, spleen, muscle, brain, pancreas, kidney, intestine, heart, lung, eye, male and female reproductive tissue) or through whole body scans. Additional tissue sections are processed for isolation of DNA and RNA for analysis of vector sequences and corresponding transcripts. Design of oligos specific for each immunoreactive polypeptide, cytokine, and shutoff transcript permit assessment of individual gene expression in all tissues. Some promoters are more actively transcribed in some tissues than others, requiring assessment of both the preferential localization of MSC to peripheral tissues after injection and MSC residency and the corresponding transcriptional activity of the recombinant genes. To this end, two artificial “barcode” nucleic acids tags can be included, one specific to Tet on/off-driven RNA transcripts, and the other to episomal vector DNA. These tags permit rapid identification of the very unique sequences among the NHP and human genome and transcriptome background.

MSC are amenable to large scale electroporation, with up to 90% efficiency. MaxCyte, Inc. (Gaithersburg, Md.) markets the MAXCYTE® VLX™ Large Scale Transfection System, a small-footprint, easy to use instrument specifically designed for extremely large volume transient transfection in a sterile, closed transfection environment. Using flow electroporation technology, the MAXCYTE® VLX™ Large Scale Transfection System can transfect up to about 2×10¹¹ cells in less than about 30 minutes with high cell viability and transfection efficiencies in a sterile, closed transfection environment. This cGMP-compliant system is useful for the rapid production of recombinant proteins, from the bench through cGMP pilots and commercial manufacturing”. MSC can be grown in chemically defined (CD) media, in large scale cell culture environments. Recent advances in bioprocessing engineering have resulted in rapid development of CD formulations that support large scale expansion of MSC without loss of pluripotent characteristics and retention of genetic stability. Adipose-derived MSC can be readily procured from liposuction procedures, with an average procedure yielding about 1×10⁸ MSC, thus providing sufficient cell numbers for expansion ex vivo prior to banking (approximately 25 doublings, >3×10¹⁵ cells) with remaining lifespan and number of doublings (approximately 25) sufficient to sustain expression and delivery of therapeutic molecules in vivo for several weeks after infusion. MSC commonly display doubling rates in the 48- to 72-hour range, thus providing in vivo lifespans in the range of 50 to 75 days. The turnover rate of infused MSC can be assessed by measuring circulating levels of transgene products, and by detection of EBV sequences by qPCR in blood, nasal aspirates, and urine, in humans. Essentially complete elimination of MSC after the desired therapeutic timespan can be achieved by inducing self-destruction via controlled inducible expression of pro-apoptotic genes built into the expression vector. Levels of circulating MSC-derived immunoreactive polypeptides or other immunomodulators after injection, and vector induced autoimmunity or GVHD responses in NHP also can be assessed. In humans, additional markers associated with autoimmune or allogeneic immune responses can be measured, such as biomarkers of liver injury (ALT, AST), liver (ALB, BIL, GGT, ALP, etc.) and renal function markers (BUN, CRE, urea, electrolytes, etc.

The lack of expression of lymphohematopoietic lineage antigens distinguishes MSCs from hematopoietic cells, endothelial cells, endothelial progenitors, monocytes, B cells and erythroblasts. Primary MSC are not immortal and thus are subject to the “Hayflick limit” of about 50 divisions for primary cells. Nevertheless, the capacity for expansion is enormous, with one cell capable of producing up to about 10¹⁵ daughter cells. Additionally, MSC have low batch-to-batch variability. Cell bank sizes capable of rapidly protecting millions of at-risk individuals can be generated by pooling large numbers of pre-screened donor adipose tissue-derived MSC: 100 donors at 1×10⁸ cells/donor×25 generations ex vivo=about 3×10¹⁷ cells; at about 1×10¹¹ cells/infusion=about 3 million doses. Two approaches can be used in the generation of therapeutic MSC banks (1) isolation, expansion, testing, banking, following by transfection, recovery and administration; and (2) isolation, expansion, testing, transfection, banking to generate ready-to-administer cells upon thawing and short recovery.

For characterization, the master cell bank can be tested for sterility, mycoplasma, in vitro and in vivo adventitious agent testing, retrovirus testing, cell identity, electron microscopy, and a number of specific virus PCR assays (the FDA requires 14 in their 1993 and 1997 guidance documents, and that list has been augmented with several recommended viruses in addition, mainly polyoma viruses). With the potential initial use of serum in primary culture conditions, testing can be performed for the 9CFR panel of bovine viruses. If cells come in contact with porcine products during normal manipulations testing for porcine viruses preferably is performed, as well.

One of the limitations of using MSC for tissue repair has been the inability of cells to permanently colonize organs after ex vivo expansion and reinjection into the person from which they were derived. MSC circulate for a limited period of time (e.g., several weeks or months), whether injected into MHC matched or unmatched individuals. This particular short-coming in the development of an adult MSC universal gene delivery platform is a benefit in the methods described herein. The pharmacokinetic (PK) profile of each transgene expressed in transfected MSC can be assessed in NHP for each engineered delivery vector platform developed. One single dose PK study desirably is performed in cynomolgus monkeys, with transfected MSC administered IV. In such a study 2 male and 2 female monkeys each are intravenously (i.v.) administered a high dose (about 10¹¹ cells), intermediate dose (about 10⁸ cells), and a low dose (about 10⁵ cells) of MSC. Endpoints to be evaluated include: cage-side observations, body weight, qualitative food consumption, ophthalmology, electrocardiogram, clinical pathology (e.g., hematology, chemistry, coagulation, urinalysis); immunology (e.g., immunoglobulins and peripheral leukocytes such as B cells, T cells and monocytes); immunogenicity; gross pathology (e.g., necropsy and selected organ weights); histopathology; tissue binding; and pharmacokinetics. Serum concentrations of each recombinant antibody can be monitored over 9 weeks with qualified sandwich type ELISA that utilize antibody-specific capture and detection (HRP-labeled anti-id) reagents on days 1, 3, 6, 12, 24, 36, 48, and 63. PK analyses can be conducted by non-compartmental methods using WINNONLIN software (Pharsight Corp). Pharmacokinetic parameters for each antibody can be expressed as maximum serum concentration (Cₘₐₓ), dose normalized serum concentration (Cₘₐₓ/D), area under the concentration-time curve from time 0 to infinity (AUC_{0-∞}), dose normalized area under the concentration-time curve from time 0 to infinity (AUC_{0-∞}/D), total body clearance (CL), volume of distribution at steady state (Vₛₛ), apparent volume of distribution during the terminal phase (V_{z}), terminal elimination phase half-life (t_{1/2,term}), and mean residence time (MRT). Peripheral circulation and compartmentalization of injected MSC can be assessed in NHP by tracking fluorescently labeled cells preloaded with cell membrane permeable CMFDA or CMTMR dyes, as described above, on freshly prepared tissue sections or through whole body scans. Vector DNA sequences and transcripts can be monitored by qPCR, as outlined above.

There is an extensive body of literature outlining the lack of rejection against MSC in vivo. Nonetheless, this phenomenon can be evaluated in NHP with multiple injections of syngeneic MSC modified with homologous and heterologous DNA vectors, followed by immunological profiling of allogeneic responses. For example, one group of NHP can be injected with a bolus of syngeneic MSC transfected with an episomal vector expressing LASV antibodies, and another with a similar vector expressing influenza antibodies. The immune response to the MSC platform and to components of the vector can be assessed weekly over the course of 77 days, during which any immunological response should be detectable. Safety and immunogenicity in NHP following activation of the shutoff mechanism by administration of doxycycline or other tetracycline analogs can be assessed in similar fashion. Following administration of a doxycycline regimen, adverse immunological responses to vector components and the MSC delivery platform can be assessed in a similar fashion, e.g., first semi-daily for the first 2 weeks, then weekly for an additional 77 days. Additional markers of apoptotic cell death can be tracked by established assays, such as increased serum lactic dehydrogenase (LDH) and caspases, and phosphatidyl serine (PS) in circulating MSC. If an immunological response to vector and MSC is not detectable following this 77-day period NHP can be re-injected with homologous MSC, one group with MSC transfected with a homologous vector, whereas the other group will receive a heterologous DNA vector. The homologous and heterologous vectors will have the same background, but with different recombinant antibody repertoires. This approach can demonstrate immunogenicity against the MSC and the expression DNA vector, irrespective of the recombinant antibody repertoire. The 77-day timeline for assessment of immunological reactions against the MSC platform is chosen based on multiple dose toxicokinetic studies with human antibodies in cynomolgus monkeys showing a mean 5000-fold reduction in peak serum levels of recombinant antibody administered at 10 mg/Kg over this time frame. In such studies some NHP can develop anti-human antibody responses around 50 to 60 days following the first administration, while some animals may never develop a detectable humoral response to the heterologous IgG.

Desirably, the MSC can be transported in a device that allows for warm chain (37° C.) transport of genetically modified MSC allowing for elimination of cold-chain transport, with increased sample capacity and cell monitoring technologies, such as devices from MicroQ Technologies. These devices maintain precise warm temperatures from about 24 to about 168 hours, thereby allowing sufficient time for deployment of a ready-to-use therapeutic anywhere in the world. Additional capacity for storage and transport of encapsulated cells can be introduced, and capsules capable of supporting gas exchange can be prepared, as needed. The elapsed time from encapsulation to administration will account for metabolic changes in IP-MSC, cell growth rate, changes in viability, and any additional product changes that will impact performance.

D. ADCC

Antibody-dependent cell-mediated cytotoxicity (ADCC) is an immune mechanism leading to the lysis of antibody-coated target cells by immune effector cells. The target cells can be cells to which antibodies or fragments thereof comprising an Fc region specifically bind, generally via the protein part that is N-terminal to the Fc region. An antibody having increased/reduced antibody dependent cell-mediated cytotoxicity (ADCC) can comprise an antibody having increased/reduced ADCC as determined by any suitable method known to those of ordinary skill in the art.

As used herein, the term “increased/reduced ADCC” can mean an increase/reduction in the number of target cells that are lysed in a given time, at a given concentration of antibody in the medium surrounding the target cells, by the mechanism of ADCC described above, or a reduction/increase in the concentration of antibody, in the medium surrounding the target cells, required to achieve the lysis of a given number of target cells in a given time, by the mechanism of ADCC. The increase/reduction in ADCC is relative to the ADCC mediated by the same antibody produced by the same type of host cells, using the same standard production, purification, formulation and storage methods (which are known to those skilled in the art), but that has not been engineered. For example, the increase in ADCC mediated by an antibody produced by host cells engineered to have an altered pattern of glycosylation (e.g., to express the glycosyltransferase, GnTIII, or other glycosyltransferases) by the methods described herein, is relative to the ADCC mediated by the same antibody produced by the same type of non-engineered host cells.

E. CDC

Complement-dependent cytotoxicity (CDC) is a function of the complement system. It is the processes in the immune system that kill pathogens by damaging their membranes without the involvement of antibodies or cells of the immune system. There are three main processes. All three insert one or more membrane attack complexes (MAC) into the pathogen which cause lethal colloid-osmotic swelling, i.e., CDC. It is one of the mechanisms by which antibodies or antibody fragments have an anti-fungal effect.

### IV. ANTIBODY CONJUGATES

Antibodies of the present disclosure can be linked to at least one agent to form an antibody conjugate. In order to increase the efficacy of antibody molecules as diagnostic or therapeutic agents, it is conventional to link or covalently bind or complex at least one desired molecule or moiety. Such a molecule or moiety can include, but is not limited to, at least one effector or reporter molecule. Effector molecules comprise molecules having a desired activity, e.g., cytotoxic activity. Non-limiting examples of effector molecules which have been attached to antibodies include toxins, anti-tumor agents, therapeutic enzymes, radionuclides, antiviral agents, chelating agents, cytokines, growth factors, and oligo- or polynucleotides. By contrast, a reporter molecule can be any moiety which can be detected using an assay. Non-limiting examples of reporter molecules which have been conjugated to antibodies include enzymes, radiolabels, haptens, fluorescent labels, phosphorescent molecules, chemiluminescent molecules, chromophores, photoaffinity molecules, colored particles or ligands, such as biotin.

Antibody conjugates are generally preferred for use as diagnostic agents. Antibody diagnostics generally fall within two classes, those for use in in vitro diagnostics, such as in a variety of immunoassays, and those for use in vivo diagnostic protocols, generally known as “antibody-directed imaging.” Many appropriate imaging agents are known in the art, as are methods for their attachment to antibodies (see, for e.g., U.S. Pat. Nos. 5,021,236, 4,938,948, and 4,472,509). The imaging moieties used can be paramagnetic ions, radioactive isotopes, fluorochromes, NMR-detectable substances, and X-ray imaging agents.

In the case of paramagnetic ions, one might mention by way of example ions such as chromium (III), manganese (II), iron (III), iron (II), cobalt (II), nickel (II), copper (II), neodymium (III), samarium (III), ytterbium (III), gadolinium (III), vanadium (II), terbium (III), dysprosium (III), holmium (III) and/or erbium (III), with gadolinium being particularly preferred. Ions useful in other contexts, such as X-ray imaging, include but are not limited to lanthanum (III), gold (III), lead (II), and especially bismuth (III).

In the case of radioactive isotopes for therapeutic and/or diagnostic application, one might mention astatine²¹¹, ¹⁴carbon, ⁵¹chromium, ³⁶chlorine, ⁵⁷cobalt, ⁵⁸cobalt, copper⁶⁷, ¹⁵²Eu, gallium⁶⁷, ³hydrogen, iodine¹²³, iodine¹²⁵, iodine¹³¹, indium¹¹¹, ⁵⁹iron, ³²phosphorus, rhenium¹⁸⁶, rhenium¹⁸⁸, ⁷⁵selenium, ³⁵sulphur, technicium^{99m} and/or yttrium⁹⁰. ¹²⁵I is often being preferred for use in certain embodiments, and technicium^{99m} and/or indium¹¹¹ are also often preferred due to their low energy and suitability for long range detection. Radioactively labeled monoclonal antibodies of the present disclosure can be produced according to well-known methods in the art. For instance, monoclonal antibodies can be iodinated by contact with sodium and/or potassium iodide and a chemical oxidizing agent such as sodium hypochlorite, or an enzymatic oxidizing agent, such as lactoperoxidase. Monoclonal antibodies according to the disclosure can be labeled with technetium^{99m} by ligand exchange process, for example, by reducing pertechnate with stannous solution, chelating the reduced technetium onto a Sephadex column and applying the antibody to this column. Alternatively, direct labeling techniques can be used, e.g., by incubating pertechnate, a reducing agent such as SNCl₂, a buffer solution such as sodium-potassium phthalate solution, and the antibody. Intermediary functional groups which are often used to bind radioisotopes which exist as metallic ions to antibody are diethylenetriaminepentaacetic acid (DTPA) or ethylene diaminetetracetic acid (EDTA).

Non-limiting examples of fluorescent labels for use as conjugates include Alexa 350, Alexa 430, AMCA, BODIPY 630/650, BODIPY 650/665, BODIPY-FL, BODIPY-R6G, BODIPY-TMR, BODIPY-TRX, Cascade Blue, Cy3, Cy5,6-FAM, Fluorescein Isothiocyanate, HEX, 6-JOE, Oregon Green 488, Oregon Green 500, Oregon Green 514, Pacific Blue, REG, Rhodamine Green, Rhodamine Red, Renographin, ROX, TAMRA, TET, Tetramethylrhodamine, and/or Texas Red.

Additional types of antibodies according to the present disclosure are those intended primarily for use in vitro, where the antibody is linked to a secondary binding ligand and/or to an enzyme (an enzyme tag) that will generate a colored product upon contact with a chromogenic substrate. Examples of suitable enzymes include urease, alkaline phosphatase, (horseradish) hydrogen peroxidase or glucose oxidase. Preferred secondary binding ligands are biotin and avidin and streptavidin compounds. The use of such labels is well known to those of skill in the art and are described, for example, in U.S. Pat. Nos. 3,817,837, 3,850,752, 3,939,350, 3,996,345, 4,277,437, 4,275,149 and 4,366,241.

Yet another known method of site-specific attachment of molecules to antibodies comprises the reaction of antibodies with hapten-based affinity labels. Essentially, hapten-based affinity labels react with amino acids in the antigen binding site, thereby destroying this site and blocking specific antigen reaction. However, this may not be advantageous since it results in loss of antigen binding by the antibody conjugate.

Molecules containing azido groups can also be used to form covalent bonds to proteins through reactive nitrene intermediates that are generated by low intensity ultraviolet light (Potter and Haley, 1983). In particular, 2- and 8-azido analogues of purine nucleotides have been used as site-directed photoprobes to identify nucleotide binding proteins in crude cell extracts (Owens & Haley, 1987; Atherton et al., 1985). The 2- and 8-azido nucleotides have also been used to map nucleotide binding domains of purified proteins (Khatoon et al., 1989; King et al., 1989; Dholakia et al., 1989) and can be used as antibody binding agents.

Several methods are known in the art for the attachment or conjugation of an antibody to its conjugate moiety. Some attachment methods involve the use of a metal chelate complex employing, for example, an organic chelating agent such a diethylenetriaminepentaacetic acid anhydride (DTPA); ethylenetriaminetetraacetic acid; N-chloro-p-toluenesulfonamide; and/or tetrachloro-3α-6α-diphenylglycouril-3 attached to the antibody (U.S. Pat. Nos. 4,472,509 and 4,938,948). Monoclonal antibodies can also be reacted with an enzyme in the presence of a coupling agent such as glutaraldehyde or periodate. Conjugates with fluorescein markers are prepared in the presence of these coupling agents or by reaction with an isothiocyanate. In U.S. Pat. No. 4,938,948, imaging of breast tumors is achieved using monoclonal antibodies and the detectable imaging moieties are bound to the antibody using linkers such as methyl-p-hydroxybenzimidate or N-succinimidyl-3-(4-hydroxyphenyl)propionate.

In other embodiments, derivatization of immunoglobulins by selectively introducing sulfhydryl groups in the Fc region of an immunoglobulin, using reaction conditions that do not alter the antibody combining site are also useful. Antibody conjugates produced according to this methodology are disclosed to exhibit improved longevity, specificity and sensitivity (U.S. Pat. No. 5,196,066, incorporated herein by reference). Site-specific attachment of effector or reporter molecules, wherein the reporter or effector molecule is conjugated to a carbohydrate residue in the Fc region have also been disclosed in the literature (O'Shannessy et al., 1987). This approach has been reported to produce diagnostically and therapeutically promising antibodies which are currently in clinical evaluation.

### V. IMMUNODETECTION METHODS

In still further embodiments, the present disclosure concerns immunodetection methods for binding, purifying, removing, quantifying and otherwise generally detecting Candida and its associated antigens. While such methods can be applied in a traditional sense, another use will be in quality control and monitoring of vaccine and other Candida stocks, where antibodies according to the present disclosure can be used to assess the amount or integrity (i.e., long term stability) of antigens in viruses. Alternatively, the methods can be used to screen various antibodies for appropriate/desired reactivity profiles.

Other immunodetection methods include specific assays for determining the presence of Candida in a subject. A wide variety of assay formats can be used, but specifically those that would be used to detect Candida in a fluid obtained from a subject, such as saliva, blood, plasma, sputum, semen or urine. In particular, semen has been demonstrated as a viable sample for detecting Candida (Purpura et al., 2016; Mansuy et al., 2016; Barzon et al., 2016; Gornet et al., 2016; Duffy et al., 2009; CDC, 2016; Halfon et al., 2010; Elder et al. 2005). The assays can be advantageously formatted for non-healthcare (home) use, including lateral flow assays (see below) analogous to home pregnancy tests. These assays can be packaged in the form of a kit with appropriate reagents and instructions to permit use by the subject of a family member.

Some immunodetection methods include enzyme linked immunosorbent assay (ELISA), radioimmunoassay (RIA), immunoradiometric assay, fluoroimmunoassay, chemiluminescent assay, bioluminescent assay, and Western blot to mention a few. In particular, a competitive assay for the detection and quantitation of Candida antibodies directed to specific parasite epitopes in samples also is provided. The steps of various useful immunodetection methods have been described in the scientific literature, such as, e.g., Doolittle and Ben-Zeev (1999), Gulbis and Galand (1993), De Jager et al. (1993), and Nakamura et al. (1987). In general, the immunobinding methods include obtaining a sample suspected of containing Candida and contacting the sample with a first antibody in accordance with the present disclosure, as the case may be, under conditions effective to allow the formation of immunocomplexes.

These methods include methods for purifying Candida or related antigens from a sample. The antibody will preferably be linked to a solid support, such as in the form of a column matrix, and the sample suspected of containing the Candida or antigenic component will be applied to the immobilized antibody. The unwanted components will be washed from the column, leaving the Candida antigen immunocomplexed to the immobilized antibody, which is then collected by removing the organism or antigen from the column.

The immunobinding methods also include methods for detecting and quantifying the amount of Candida or related components in a sample and the detection and quantification of any immune complexes formed during the binding process. Here, one would obtain a sample suspected of containing Candida or its antigens and contact the sample with an antibody that binds Candida or components thereof, followed by detecting and quantifying the amount of immune complexes formed under the specific conditions. In terms of antigen detection, the biological sample analyzed can be any sample that is suspected of containing Candida or Candida antigen, such as a tissue section or specimen, a homogenized tissue extract, a biological fluid, including blood and serum, or a secretion, such as feces or urine.

Contacting the chosen biological sample with the antibody under effective conditions and for a period of time sufficient to allow the formation of immune complexes (primary immune complexes) is generally a matter of simply adding the antibody composition to the sample and incubating the mixture for a period of time long enough for the antibodies to form immune complexes with, i.e., to bind to Candida or antigens present. After this time, the sample-antibody composition, such as a tissue section, ELISA plate, dot blot or Western blot, will generally be washed to remove any non-specifically bound antibody species, allowing only those antibodies specifically bound within the primary immune complexes to be detected.

In general, the detection of immunocomplex formation is well known in the art and can be achieved through the application of numerous approaches. These methods are generally based upon the detection of a label or marker, such as any of those radioactive, fluorescent, biological and enzymatic tags. Patents concerning the use of such labels include U.S. Pat. Nos. 3,817,837, 3,850,752, 3,939,350, 3,996,345, 4,277,437, 4,275,149 and 4,366,241. Of course, one may find additional advantages through the use of a secondary binding ligand such as a second antibody and/or a biotin/avidin ligand binding arrangement, as is known in the art.

The antibody employed in the detection can itself be linked to a detectable label, wherein one would then simply detect this label, thereby allowing the amount of the primary immune complexes in the composition to be determined. Alternatively, the first antibody that becomes bound within the primary immune complexes can be detected by means of a second binding ligand that has binding affinity for the antibody. In these cases, the second binding ligand can be linked to a detectable label. The second binding ligand is itself often an antibody, which can thus be termed a “secondary” antibody. The primary immune complexes are contacted with the labeled, secondary binding ligand, or antibody, under effective conditions and for a period of time sufficient to allow the formation of secondary immune complexes. The secondary immune complexes are then generally washed to remove any non-specifically bound labeled secondary antibodies or ligands, and the remaining label in the secondary immune complexes is then detected.

Further methods include the detection of primary immune complexes by a two-step approach. A second binding ligand, such as an antibody that has binding affinity for the antibody, is used to form secondary immune complexes, as described above. After washing, the secondary immune complexes are contacted with a third binding ligand or antibody that has binding affinity for the second antibody, again under effective conditions and for a period of time sufficient to allow the formation of immune complexes (tertiary immune complexes). The third ligand or antibody is linked to a detectable label, allowing detection of the tertiary immune complexes thus formed. This system can provide for signal amplification if this is desired.

One method of immunodetection uses two different antibodies. A first biotinylated antibody is used to detect the target antigen, and a second antibody is then used to detect the biotin attached to the complexed biotin. In that method, the sample to be tested is first incubated in a solution containing the first step antibody. If the target antigen is present, some of the antibody binds to the antigen to form a biotinylated antibody/antigen complex. The antibody/antigen complex is then amplified by incubation in successive solutions of streptavidin (or avidin), biotinylated DNA, and/or complementary biotinylated DNA, with each step adding additional biotin sites to the antibody/antigen complex. The amplification steps are repeated until a suitable level of amplification is achieved, at which point the sample is incubated in a solution containing the second step antibody against biotin. This second step antibody is labeled, as for example with an enzyme that can be used to detect the presence of the antibody/antigen complex by histoenzymology using a chromogen substrate. With suitable amplification, a conjugate can be produced which is macroscopically visible.

Another known method of immunodetection takes advantage of the immuno-PCR (Polymerase Chain Reaction) methodology. The PCR method is similar to the Cantor method up to the incubation with biotinylated DNA, however, instead of using multiple rounds of streptavidin and biotinylated DNA incubation, the DNA/biotin/streptavidin/antibody complex is washed out with a low pH or high salt buffer that releases the antibody. The resulting wash solution is then used to carry out a PCR reaction with suitable primers with appropriate controls. At least in theory, the enormous amplification capability and specificity of PCR can be utilized to detect a single antigen molecule.

A. ELISAs

Immunoassays, in their most simple and direct sense, are binding assays. Certain preferred immunoassays are the various types of enzyme linked immunosorbent assays (ELISAs) and radioimmunoassays (RIA) known in the art. Immunohistochemical detection using tissue sections is also particularly useful. However, it will be readily appreciated that detection is not limited to such techniques, and western blotting, dot blotting, FACS analyses, and the like can also be used.

In one exemplary ELISA, the antibodies of the disclosure are immobilized onto a selected surface exhibiting protein affinity, such as a well in a polystyrene microliter plate. Then, a test composition suspected of containing the Candida or Candida antigen is added to the wells. After binding and washing to remove non-specifically bound immune complexes, the bound antigen can be detected. Detection can be achieved by the addition of another anti-Candida antibody that is linked to a detectable label. This type of ELISA is a simple “sandwich ELISA.” Detection can also be achieved by the addition of a second anti-Candida antibody, followed by the addition of a third antibody that has binding affinity for the second antibody, with the third antibody being linked to a detectable label.

In another exemplary ELISA, the samples suspected of containing the Candida or Candida antigen are immobilized onto the well surface and then contacted with the anti-Candida antibodies of the disclosure. After binding and washing to remove non-specifically bound immune complexes, the bound anti-Candida antibodies are detected. Where the initial anti-Candida antibodies are linked to a detectable label, the immune complexes can be detected directly. Again, the immune complexes can be detected using a second antibody that has binding affinity for the first anti-Candida antibody, with the second antibody being linked to a detectable label.

Irrespective of the format employed, ELISAs have certain features in common, such as coating, incubating and binding, washing to remove non-specifically bound species, and detecting the bound immune complexes. These are described below.

In coating a plate with either antigen or antibody, one will generally incubate the wells of the plate with a solution of the antigen or antibody, either overnight or for a specified period of hours. The wells of the plate will then be washed to remove incompletely adsorbed material. Any remaining available surfaces of the wells are then “coated” with a nonspecific protein that is antigenically neutral with regard to the test antisera. These include bovine serum albumin (BSA), casein or solutions of milk powder. The coating allows for blocking of nonspecific adsorption sites on the immobilizing surface and thus reduces the background caused by nonspecific binding of antisera onto the surface.

In ELISAs, it is probably more customary to use a secondary or tertiary detection means rather than a direct procedure. Thus, after binding of a protein or antibody to the well, coating with a non-reactive material to reduce background, and washing to remove unbound material, the immobilizing surface is contacted with the biological sample to be tested under conditions effective to allow immune complex (antigen/antibody) formation. Detection of the immune complex then requires a labeled secondary binding ligand or antibody, and a secondary binding ligand or antibody in conjunction with a labeled tertiary antibody or a third binding ligand.

“Under conditions effective to allow immune complex (antigen/antibody) formation” means that the conditions preferably include diluting the antigens and/or antibodies with solutions such as BSA, bovine gamma globulin (BGG) or phosphate buffered saline (PBS)/Tween. These added agents also tend to assist in the reduction of nonspecific background.

The “suitable” conditions also mean that the incubation is at a temperature or for a period of time sufficient to allow effective binding. Incubation steps are typically from about 1 to 2 to 4 hours or so, at temperatures preferably on the order of 25° C. to 27° C. or can be overnight at about 4° C. or so.

Following all incubation steps in an ELISA, the contacted surface is washed so as to remove non-complexed material. A preferred washing procedure includes washing with a solution such as PBS/Tween, or borate buffer. Following the formation of specific immune complexes between the test sample and the originally bound material, and subsequent washing, the occurrence of even minute amounts of immune complexes can be determined.

To provide a detecting means, the second or third antibody will have an associated label to allow detection. Preferably, this will be an enzyme that will generate color development upon incubating with an appropriate chromogenic substrate. Thus, for example, one will desire to contact or incubate the first and second immune complex with a urease, glucose oxidase, alkaline phosphatase or hydrogen peroxidase-conjugated antibody for a period of time and under conditions that favor the development of further immune complex formation (e.g., incubation for 2 hours at room temperature in a PBS-containing solution such as PBS-Tween).

After incubation with the labeled antibody, and subsequent to washing to remove unbound material, the amount of label is quantified, e.g., by incubation with a chromogenic substrate such as urea, or bromocresol purple, or 2,2′-azino-di-(3-ethyl-benzthiazoline-6-sulfonic acid (ABTS), or H₂O₂, in the case of peroxidase as the enzyme label. Quantification is then achieved by measuring the degree of color generated, e.g., using a visible spectra spectrophotometer.

In another embodiment, the present disclosure is directed to the use of competitive formats. This is particularly useful in the detection of Candida antibodies in sample. In competition-based assays, an unknown amount of analyte or antibody is determined by its ability to displace a known amount of labeled antibody or analyte. Thus, the quantifiable loss of a signal is an indication of the amount of unknown antibody or analyte in a sample.

Here, the inventor proposes the use of labeled Candida monoclonal antibodies to determine the amount of Candida antibodies in a sample. The basic format would include contacting a known amount of Candida monoclonal antibody (linked to a detectable label) with Candida antigen or particle. The Candida antigen or organism is preferably attached to a support. After binding of the labeled monoclonal antibody to the support, the sample is added and incubated under conditions permitting any unlabeled antibody in the sample to compete with, and hence displace, the labeled monoclonal antibody. By measuring either the lost label or the label remaining (and subtracting that from the original amount of bound label), one can determine how much non-labeled antibody is bound to the support, and thus how much antibody was present in the sample.

B. Western Blot

The Western blot (alternatively, protein immunoblot) is an analytical technique used to detect specific proteins in a given sample of tissue homogenate or extract. It uses gel electrophoresis to separate native or denatured proteins by the length of the polypeptide (denaturing conditions) or by the 3-D structure of the protein (native/non-denaturing conditions). The proteins are then transferred to a membrane (typically nitrocellulose or PVDF), where they are probed (detected) using antibodies specific to the target protein.

Samples can be taken from whole tissue or from cell culture. In most cases, solid tissues are first broken down mechanically using a blender (for larger sample volumes), using a homogenizer (smaller volumes), or by sonication. Cells can also be broken open by one of the above mechanical methods. However, it should be noted that environmental samples can be the source of protein and thus Western blotting is not restricted to cellular studies only. Assorted detergents, salts, and buffers can be employed to encourage lysis of cells and to solubilize proteins. Protease and phosphatase inhibitors are often added to prevent the digestion of the sample by its own enzymes. Tissue preparation is often done at cold temperatures to avoid protein denaturing.

The proteins of the sample are separated using gel electrophoresis. Separation of proteins can be by isoelectric point (pI), molecular weight, electric charge, or a combination of these factors. The nature of the separation depends on the treatment of the sample and the nature of the gel. This is a very useful way to determine a protein. Two-dimensional (2-D) gel can also be used, which spreads the proteins from a single sample out in two dimensions. Proteins are separated according to isoelectric point (pH at which they have neutral net charge) in the first dimension, and according to their molecular weight in the second dimension.

In order to make the proteins accessible to antibody detection, they are moved from within the gel onto a membrane made of nitrocellulose or polyvinylidene difluoride (PVDF). The membrane is placed on top of the gel, and a stack of filter papers placed on top of that. The entire stack is placed in a buffer solution which moves up the paper by capillary action, bringing the proteins with it. Another method for transferring the proteins is called electroblotting and uses an electric current to pull proteins from the gel into the PVDF or nitrocellulose membrane. The proteins move from within the gel onto the membrane while maintaining the organization they had within the gel. As a result of this blotting process, the proteins are exposed on a thin surface layer for detection (see below). Both varieties of membrane are chosen for their non-specific protein binding properties (i.e., binds all proteins equally well). Protein binding is based upon hydrophobic interactions, as well as charged interactions between the membrane and protein. Nitrocellulose membranes are cheaper than PVDF but are far more fragile and do not stand up well to repeated probings. The uniformity and overall effectiveness of transfer of protein from the gel to the membrane can be checked by staining the membrane with Coomassie Brilliant Blue or Ponceau S dyes. Once transferred, proteins are detected using labeled primary antibodies, or unlabeled primary antibodies followed by indirect detection using labeled protein A or secondary labeled antibodies binding to the Fc region of the primary antibodies.

C. Lateral Flow Assays

Lateral flow assays, also known as lateral flow immunochromatographic assays, are simple devices intended to detect the presence (or absence) of a target analyte in sample (matrix) without the need for specialized and costly equipment, though many laboratory-based applications exist that are supported by reading equipment. Typically, these tests are used as low resources medical diagnostics, either for home testing, point of care testing, or laboratory use. A widely spread and well-known application is the home pregnancy test.

The technology is based on a series of capillary beds, such as pieces of porous paper or sintered polymer. Each of these elements has the capacity to transport fluid (e.g., urine) spontaneously. The first element (the sample pad) acts as a sponge and holds an excess of sample fluid. Once soaked, the fluid migrates to the second element (conjugate pad) in which the manufacturer has stored the so-called conjugate, a dried format of bio-active particles (see below) in a salt-sugar matrix that contains everything to guarantee an optimized chemical reaction between the target molecule (e.g., an antigen) and its chemical partner (e.g., antibody) that has been immobilized on the particle's surface. While the sample fluid dissolves the salt-sugar matrix, it also dissolves the particles and in one combined transport action the sample and conjugate mix while flowing through the porous structure. In this way, the analyte binds to the particles while migrating further through the third capillary bed. This material has one or more areas (often called stripes) where a third molecule has been immobilized by the manufacturer. By the time the sample-conjugate mix reaches these strips, analyte has been bound on the particle and the third ‘capture’ molecule binds the complex. After a while, when more and more fluid has passed the stripes, particles accumulate and the stripe-area changes color. Typically, there are at least two stripes: one (the control) that captures any particle and thereby shows that reaction conditions and technology worked fine, the second contains a specific capture molecule and only captures those particles onto which an analyte molecule has been immobilized. After passing these reaction zones, the fluid enters the final porous material—the wick—that simply acts as a waste container. Lateral Flow Tests can operate as either competitive or sandwich assays. Lateral flow assays are disclosed in U.S. Pat. No. 6,485,982.

D. Immunohistochemistry

The antibodies of the present disclosure can also be used in conjunction with both fresh-frozen and/or formalin-fixed, paraffin-embedded tissue blocks prepared for study by immunohistochemistry (IHC). The method of preparing tissue blocks from these particulate specimens has been successfully used in previous IHC studies of various prognostic factors and is well known to those of skill in the art (Brown et al., 1990; Abbondanzo et al., 1990; Allred et al., 1990).

Briefly, frozen-sections can be prepared by rehydrating 50 ng of frozen “pulverized” tissue at room temperature in phosphate buffered saline (PBS) in small plastic capsules; pelleting the particles by centrifugation; resuspending them in a viscous embedding medium (OCT); inverting the capsule and/or pelleting again by centrifugation; snap-freezing in −70° C. isopentane; cutting the plastic capsule and/or removing the frozen cylinder of tissue; securing the tissue cylinder on a cryostat microtome chuck; and/or cutting 25-50 serial sections from the capsule. Alternatively, whole frozen tissue samples can be used for serial section cuttings.

Permanent-sections can be prepared by a similar method involving rehydration of the 50 mg sample in a plastic microfuge tube; pelleting; resuspending in 10% formalin for 4 hours fixation; washing/pelleting; resuspending in warm 2.5% agar; pelleting; cooling in ice water to harden the agar; removing the tissue/agar block from the tube; infiltrating and/or embedding the block in paraffin; and/or cutting up to 50 serial permanent sections. Again, whole tissue samples can be substituted.

E. Immunodetection Kits

In still further embodiments, the present disclosure concerns immunodetection kits for use with the immunodetection methods described above. As the antibodies can be used to detect Candida or Candida antigens, the antibodies can be included in the kit. The immunodetection kits will thus comprise, in suitable container means, a first antibody that binds to Candida or Candida antigen, and optionally an immunodetection reagent.

In certain embodiments, the Candida antibody can be pre-bound to a solid support, such as a column matrix and/or well of a microtiter plate. The immunodetection reagents of the kit can take any one of a variety of forms, including those detectable labels that are associated with or linked to the given antibody. Detectable labels that are associated with or attached to a secondary binding ligand can also be used. Exemplary secondary ligands are those secondary antibodies that have binding affinity for the first antibody.

Further suitable immunodetection reagents for use in the present kits include the two-component reagent that comprises a secondary antibody that has binding affinity for the first antibody, along with a third antibody that has binding affinity for the second antibody, the third antibody being linked to a detectable label. As noted above, a number of exemplary labels are known in the art and all such labels can be employed in connection with the present disclosure.

The kits may further comprise a suitably aliquoted composition of the Candida or Candida antigens, whether labeled or unlabeled, as may be used to prepare a standard curve for a detection assay. The kits can contain antibody-label conjugates either in fully conjugated form, in the form of intermediates, or as separate moieties to be conjugated by the user of the kit. The components of the kits can be packaged either in aqueous media or in lyophilized form.

The container means of the kits will generally include at least one vial, test tube, flask, bottle, syringe or other container means, into which the antibody can be placed, or preferably, suitably aliquoted. The kits of the present disclosure will also typically include a means for containing the antibody, antigen, and any other reagent containers in close confinement for commercial sale. Such containers can include injection or blow-molded plastic containers into which the desired vials are retained.

F. Vaccine and Antigen Quality Control Assays

The present disclosure also directed to the use of antibodies and antibody fragments as described herein for use in assessing the antigenic integrity (e.g., the ability of an antigen to exhibit a relevant or natural antigenic or immunogenic structure) of a fungal antigen in a sample. Biological medicinal products like vaccines differ from chemical drugs in that they cannot normally be characterized molecularly; antibodies are large molecules of significant complexity and have the capacity to vary widely from preparation to preparation. They are also administered to healthy individuals, including children at the start of their lives, and thus a strong emphasis must be placed on their quality to ensure, that they are efficacious in preventing or treating life-threatening disease, without themselves causing harm.

The increasing globalization in the production and distribution of vaccines has opened new possibilities to better manage public health concerns but has also raised questions about the equivalence and interchangeability of vaccines procured across a variety of sources. International standardization of starting materials, of production and quality control testing, and the setting of high expectations for regulatory oversight on the way these products are manufactured and used, have thus been the cornerstone for continued success. But it remains a field in constant change, and continuous technical advances in the field offer a promise of developing potent new weapons against the oldest public health threats, as well as new ones—malaria, pandemic influenza, and HIV, to name a few—but also put a great pressure on manufacturers, regulatory authorities, and the wider medical community to ensure that products continue to meet the highest standards of quality attainable.

Thus, one can obtain an antigen or vaccine from any source or at any point during a manufacturing process. The quality control processes can therefore begin with preparing a sample for an immunoassay that identifies binding of an antibody or fragment disclosed herein to a fungal antigen. Such immunoassays are disclosed elsewhere in this document, and any of these can be used to assess the structural/antigenic integrity of the antigen. Standards for finding the sample to contain acceptable amounts of antigenically correct and intact antigen may be established by regulatory agencies.

Another important embodiment where antigen integrity is assessed is in determining shelf-life and storage stability. Most medicines, including vaccines, can deteriorate over time. Therefore, it is critical to determine whether, over time, the degree to which an antigen, such as in a vaccine, degrades or destabilizes such that is it no longer antigenic and/or capable of generating an immune response when administered to a subject. Again, standards for finding the sample to contain acceptable amounts of antigenically intact antigen may be established by regulatory agencies.

In certain embodiments, fungal antigens can contain more than one protective epitope. In these cases, it can prove useful to employ assays that look at the binding of more than one antibody, such as 2, 3, 4, 5 or even more antibodies. These antibodies bind to closely related epitopes, such that they are adjacent or even overlap each other. On the other hand, they may represent distinct epitopes from disparate parts of the antigen. By examining the integrity of multiple epitopes, a more complete picture of the antigen's overall integrity, and hence ability to generate a protective immune response, can be determined.

Antibodies and fragments thereof as described in the present disclosure can also be used in a kit for monitoring the efficacy of vaccination procedures by detecting the presence of protective Candida antibodies. Antibodies, antibody fragment, or variants and derivatives thereof, as described in the present disclosure can also be used in a kit for monitoring vaccine manufacture with the desired immunogenicity.

### VI. EXAMPLES

The following examples are included to demonstrate preferred embodiments. It should be appreciated by those of skill in the art that the techniques disclosed in the examples that follow represent techniques discovered by the inventor to function well in the practice of embodiments, and thus can be considered to constitute preferred modes for its practice. However, those of skill in the art should, in light of the present invention, appreciate that many changes can be made in the specific embodiments which are disclosed and still obtain a like or similar result without departing from the spirit and scope of the disclosure.

### Example 1

### Presence of Antibodies to Fba and Met6 Peptides in Human Sera Samples and Molecular Cloning of Human Monoclonal Antibodies

### Materials and Methods

ELISA screening. Wells of 96 well assay plates were coated with Fba (SEQ ID NO: 40) or MET6 (SEQ ID NO: 38) peptides for 90 min at room temperature. Peptides were diluted to 1 μg/ml in 100 mM Na bicarbonate pH 9.6. Plates were then washed and blocked for 30 min with PBS (Phosphate Buffered Saline, pH 7.4) containing 0.5% Tween 20™, 4% whey proteins, and 10% fetal bovine serum (Blocking Buffer). Sera were heat inactivated and diluted in blocking buffer and tested at a 1:100 dilution. 100 μl of each serum sample was incubated for 90 min at room temperature in wells coated with or without peptides. MAbs 1.11D (anti-Fba; SEQ ID NO: 10 and SEQ ID NO: 11) and 1.10C (anti-MET6; SEQ ID NO: 12 and SEQ ID NO: 13) were used as positive controls. Wells were washed with PBS plus 0.5% Tween 20™ and incubated with a 1:2000 dilution in Blocking Buffer of HRP conjugated goat anti-human IgG (Jackson Immunoresearch) for 60 min. Wells were washed with PBS plus 0.5% Tween 20™ and color was developed with TMB (3,3′,5,5′-Tetramethylbenzidine)-H₂O₂. The reaction was stopped with 1 M Phosphoric acid, and color was read as absorbance at 450 nm.

Memory B cell Stimulation and molecular cloning of human MAbs. Memory B cells were purified by depleting PBMC of CD2+, CD14+, and CD16+ non-B cells and then positively selecting CD27+ B cells using immunomagnetic beads (Robinson et al., 2016) Memory B cells were cultured in wells of multiple-well plates containing MS40L feeder cells, Iscoves Modified Dulbecco's Medium 10% FCS, CpG, IL-2 and IL-21. MS40L were derived from a murine stromal cell line, MS5, and have been engineered to express human CD40L (Luo et al., 2009). MS40L cells support robust memory B cell growth. B cells were seeded at low cell densities to achieve near clonal stimulation of B cells in each well. At 2 weeks, culture fluids were screened by ELISA for IgG antibodies reacting with candida peptides. Cells in antibody-positive wells were harvested and stored in guanidine lysis buffer (Ambion RNAqueous isolation Kit). Next, RNA purified from B cells was reverse transcribed to make cDNA (Tiller et al., 2008). Nested PCR was then performed to amplify variable regions of heavy and light chains which were then inserted into heavy and light chain expression vectors as described (Robinson et al., 2016). Matched pairs of heavy and light chain vectors were then transfected into 293T cells. Culture supernatants were tested for peptide binding antibody after 48 hours. Cross-transfections with multiple clones of heavy and light genes from the same B cell culture were performed to ensure the products were cloned VH and VL genes. Once definitive pairs of HC and LC plasmids that make a Mab, are identified, the HC and LC genes were sequenced and small-scale antibody production in transiently transfected cultures of 293T cells was used to produce purified MAb to permit further characterization of MAb in vitro (Cortin et al, 2013; Robinson et al., 2016).

### Results and Discussion

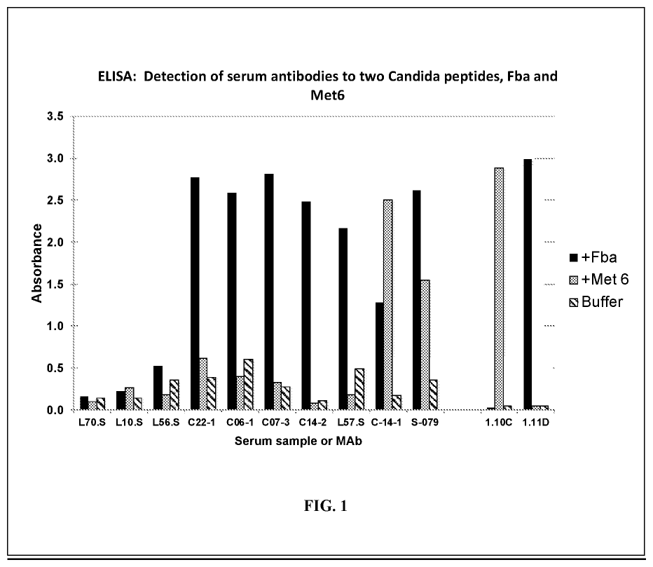

Ten human sera samples were tested for the presence of antibodies that bind to the Fba (SEQ ID NO: 40) or the MET6 (SEQ ID NO: 38) peptides. As shown in FIG. 1, several of the samples were positive for the presence of antibodies to the Fba peptide (SEQ ID NO: 40), while several were found to be positive for antibodies to both peptides demonstrating that anti-peptide antibodies recognizing the Fba peptide (SEQ ID NO: 40) or the MET6 peptide (SEQ ID NO: 38) exist in humans.

### Example 2

### Demonstration of Specific Binding of Human Monoclonal Antibodies to Fba and Met6 Peptides by Competition ELISA

### Materials and Methods

Antibody production and purification. Matched pairs of plasmids (or a dual expression plasmid) expressing the heavy and light chains of either 1.100 (anti-MET6; SEQ ID NO: 12 and SEQ ID NO: 13) or 1.11D (anti-Fba; SEQ ID NO: 10 and SEQ ID NO: 11) were transiently transfected into Freestyle™ 293 cells and the cells were incubated in Freestyle™ media at 30° C. in 8% CO₂ according to the manufacturer's instructions (Thermo Fisher Scientific). Immunoglobulin production was monitored using anti-human Bio-layer interferometry tips (ForteBio) on a BLITZ interferometer (ForteBio). Cell supernatants were harvested by centrifugation at 6,000 g for 10 min when antibody production had plateaued, and subsequently filter sterilized. IgG was purified by Fast Flow Protein G (GE Life Sciences) affinity chromatography. Cleared supernatants were applied to 1 ml columns of Fast Flow protein G sepharose using a peristaltic pump and recycled through the column 2-3 times. The columns were then washed with 10 volumes of PBS and bound IgG was eluted from the column with 0.1 M glycine buffer, pH 2.0. Eluted fractions were neutralized by the addition of 1/10^{th} volume of 1 M Tris buffer pH 8.0. The eluted protein is then concentrated using centrifugal ultrafilters (30-50,000 MWCO; Amicon) to a protein concentration of approximately 1 mg/ml, dialyzed against PBS, filter sterilized and store 4° C.

ELISA. Briefly, Fba (SEQ ID NO: 40) or MET6 peptide (SEQ ID NO: 38) was dissolved in coating buffer (4 μg/ml), and the solutions were used to coat 96-well ELISA plates (100 μl, room temperature for 1 h and overnight at 4° C.). The wells were washed two times with PBS and blocked with 1% bovine serum albumin/PBS, 200 μl). The antibodies 1.10C (anti-MET6; SEQ ID NO: 12 and SEQ ID NO: 13) or 1.11D (anti-Fba; SEQ ID NO: 10 and SEQ ID NO: 11) were mixed with the cognate peptide Fba (SEQ ID NO: 40) or MET6 (SEQ ID NO: 38) peptide (inhibitor) dissolved in PBS plus 1% BSA at a concentration between 200 μg/ml to 3.125 μg/ml. The resulting solution of each concentration was added to the Fba-coated or MET6-coated microtiter wells in triplicate and incubated at 37° C. for 2 h. The wells were washed three times with PBS plus 0.5% Tween 20™, one time with PBS, and mouse anti-human IgG HRP (Sigma, A5420) (diluted 1:3,000 in PBS plus 0.5% Tween 20™) 100 μl was added and incubated for 1 h at 37° C. The wells were washed three times with PBST, followed by addition of 100 μl of substrate solution (25 ml of 0.05 M phosphate-citrate buffer pH 5.0, 200 μl of an aqueous solution of O-phenylenediamine 50 mg/ml, Sigma, and 10 μl of 30% H₂O₂). Color was allowed to develop for 10-20 min, stopped by addition of 100 μl of 2M H₂SO₄ and read at 492 nm (microtiter plate reader, model 450; Bio-Rad, Richmond, Calif.). The percent inhibition was calculated relative to wells containing antibody without inhibitor.

### Results and Discussion

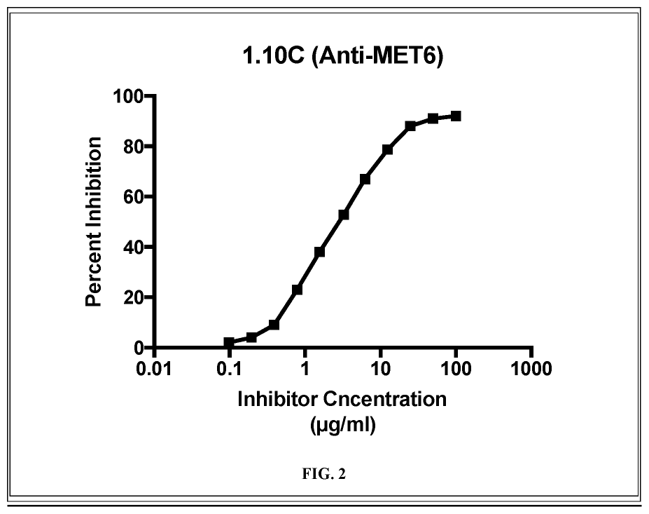

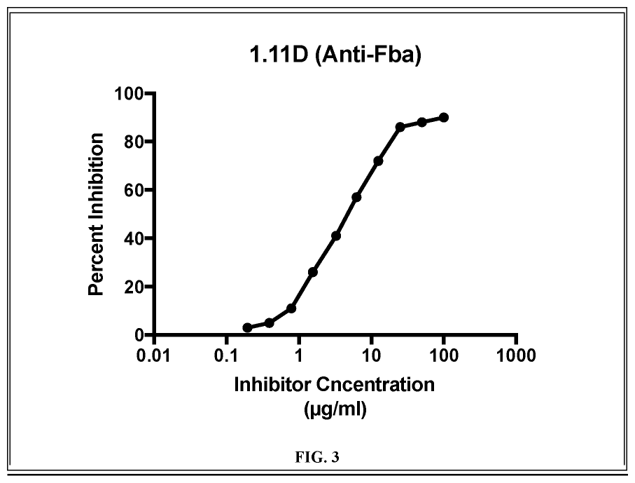

As shown in both FIG. 2 and FIG. 3 added free peptide competed with bound peptide for the binding of 1.10C (anti-MET6; SEQ ID NO: 12 and SEQ ID NO: 13) and 1.11D (anti-Fba; SEQ ID NO: 10 and SEQ ID NO: 11). These results demonstrate that the binding of the antibodies to their cognate peptides is specific.

### Example 3

### Determination of Binding Affinities of 1.10C and 1.11D to their Cognate Peptides by Bio-Layer Interferometry (BLI)

### Materials and Methods

Bio-Layer Interferometry. Binding experiments were performed on an Octet HTX at 25° C. Streptavidin (SA) biosensors were hydrated in Assay Buffer (PBS with 0.1% BSA, 0.02% Tween-20 (pH 7.4)), and biotinylated peptides (Fba-Biotin, seq ID 41; Met6-Biotin, seq ID 39) at 0.01 μg/mL in Assay Buffer were loaded onto Streptavidin (SA) biosensors. Loaded sensors were dipped into serial dilutions of the cognate IgG (purified as above in Example 2; 300 nM start, 1:3 dilution, 7 points) for 15 minutes so that the binding reached equilibrium. Kinetic constants were calculated using a monovalent (1:1) binding model. Steady-steady analyses were also used to estimate the affinity of antibody binding to cognate peptide using the following model equation:

Req=Rmax*C/(C+kD)

in which Req is the average response level between 890-895 second during association, Rmax is projected maximum response level, kD is the affinity, and C is the antibody concentration.

### Results and Discussion

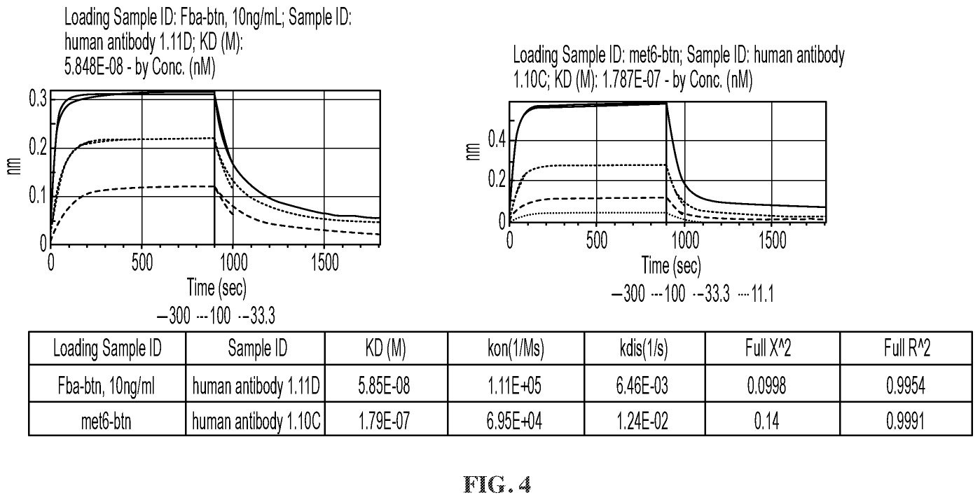

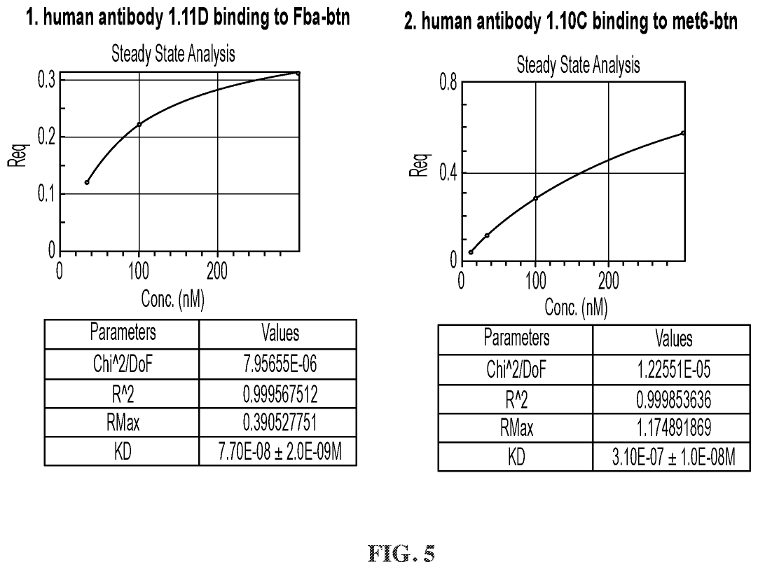

Kinetic measurements show (FIG. 4) that the human antibody 1.11D (anti-Fba; SEQ ID NO: 10 and SEQ ID NO: 11) has a kD of approximately 5.8×10⁻⁸ for binding to the Fba peptide (Fba-Biotin, SEQ ID NO: 41) while the human antibody 1.10C (anti-MET6; SEQ ID NO: 12 and SEQ ID NO: 13) has a kD of approximately 1.8×10⁻⁷ for binding to the MET6 peptide (MET6-Biotin, SEQ ID NO: 39). Steady-state measurements (FIG. 5) revealed a kD of approximately 7.7×10⁻⁸ for 1.11D (anti-Fba; SEQ ID NO: 10 and 11) binding to the Fba peptide (Fba-Biotin, SEQ ID NO: 41) and a kD of approximately 3.1×10⁻⁷ for 1.10C (anti-MET6; SEQ ID NO: 12 and SEQ ID NO: 13) binding to the MET6 peptide (MFT6-Biotin, SEQ ID NO: 39).

### Example 4

### Demonstration of Binding of 1.10C and 1.11D to the Full-Length Recombinant Proteins Met6 and Fba by Bio-Layer Interferometry

### Materials and Methods

Generation of C. albicans and C. auris full-length recombinant Fba and MET6. cDNA's for Fba were generated, as described (Li et at., 2013) and cloned using the vector pRSET A (Invitrogen). This inducible expression vector generates recombinant portions with a six-histidine (6-His) amino terminal tag. The resulting expression vector was transformed into the NiCo21(DE3) protein production strain (New England Biolabs) which is a specifically designed strain of E. coli for the expression of 6-His-tagged recombinant proteins. The MET6 gene sequence from C. albicans (NCBI Reference Sequence: XM_713126.2) was chemically synthesized (Genscript) and subcloned into pRSET A.

Production of bacterial supernatants containing full-length recombinant Fba or MET6. Overnight cultures prepared in SOB (2% w/v tryptone, 0.5% w/v yeast extract, 10 mM NaCl, 2.5 mM KCI, 10 mM MgCl₂, 10 mM MgSO₄ in H₂O₂) of NiCo21(DE3) cells transformed with either pRSET A MET6 or pRSET A Fba, grown SOB at 37° C. with shaking at 250 RPM, were diluted into at 37° C. with shaking at 250 RPM to an O.D. of 0.1 When the cultures reached an O.D. of between 0.4 and 0.6, isopropyl β-D-1-thiogalactopyranoside (IPTG) was added to 100 μM and the cultures were incubated for an additional 12 hr. Bacterial cells were then harvested by centrifugation at 6,000×g for 100 min. The culture supernatant was discarded by aspiration and the cell pellet was resuspended in CellLytic B™ (Sigma) (1 ml/25 ml of original bacterial culture). The extraction suspension was incubated at room temperature with gentle mixing for 15 min. After extraction the suspension is centrifuged at 16,000×g for 10 minutes to pellet the insoluble material. The supernatant was carefully removed, aliquoted, and frozen at −20° C. until used.

Fba BLI analysis. Detectable binding of purified human monoclonal antibody (HuMAb) to full length C. albicans and C. auris Fba protein was accomplished using the FortèBio BLITz instrument (software: BLITz Pro 1.2). Before beginning, an Anti-Penta-His sensor (HisK sensor; FortèBio) was hydrated in Kinetics Buffer (PBS+0.1% BSA+0.02% Tween20). After a baseline step in Kinetics Buffer, the full-length His-tagged Fba protein (C. albicans or C. auris) was loaded onto the sensor tip using crude bacterial expression supernatants diluted 1:1 with Kinetics Buffer. A. secondary baseline step was performed using Kinetics Buffer. Then an association step was performed with the loaded tip dipped into a solution containing purified HuMAb 1.11D (prepared as above in Example 2; anti-Fba; SEQ ID NO: 10 and SEQ ID NO: 11) in PBS w/ Kinetics Buffer (conc=100 ug/mL); positive increase in signal indicating antibody binding. Finally, the tip was transitioned back to Kinetics Buffer for a dissociation step. A negative control was run, omitting the His-Fba loading step (using non-transformed bacterial supernatant only), to show that HuMAb 1.11D (anti-Fba; SEQ ID NO: 10 and SEQ ID NO: 11) did not simply recognize the HisK sensor and that the species loaded onto the tip did not come from the bacterial supernatant. Additionally, HuMAb 1.10C (anti-MET6; SEQ ID NO: 12 and SEQ ID NO: 13) at the same concentration (100 μg/mL) was used as a negative control in the association step to show 1.11D antibody binds specificity to the Fba loaded tip.

MET6 BLI analysis. Detectable binding of purified human monoclonal antibody (HuMAb) to full length C. albicans Met6 protein (NCBI Reference Sequence: XM_713126.2) was accomplished using the ForteBio BLITz instrument (software: BLITz Pro 1.2). Before beginning, an Anti-Penta-His sensor (HisK sensor; FortèBio) was hydrated in Kinetics Buffer [PBS+0.1% BSA+0.02% Tween20]. After a baseline step in Kinetics Buffer, the full-length His-tagged Met6 protein (C. albicans) was loaded onto the sensor tip using crude bacterial expression supernatants diluted 1:1 with Kinetics Buffer. A secondary baseline step was performed using Kinetics Buffer. Then an association step was performed with the loaded tip dipped into a solution containing purified. HuMAb 1.10C (anti-MET6; SEQ ID NO: 12 and SEQ ID NO: 13) in DPBS w/ Kinetics Buffer (cone=100 ug/mL); positive growth in signal indicating antibody binding. Finally, the tip was transitioned back to Kinetics Buffer for a dissociation step. A negative control was run, omitting the His-Met6 in the loading step (using non-transformed bacterial supernatant only), to show that HuMAb 1.10C (anti-MET6; SEQ ID NO: 12 and SEQ ID NO: 13) did not simply recognize the HisK sensor and that the species loaded onto the tip did not come from the bacterial supernatant. Additionally, HuMAb 1.11D (anti-Fba; SEQ ID NO: 10 and SEQ ID NO: 11), at the same concentration (100 μg/mL), was used as a negative control in the association step to show 1.10C antibody specificity to the Met6 loaded tip.

### Results and Discussion

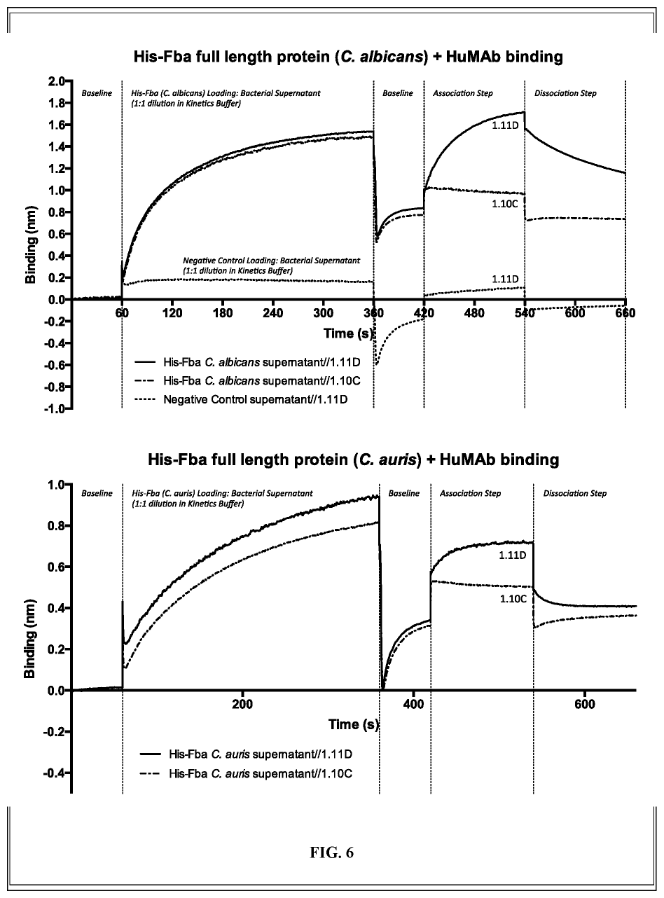

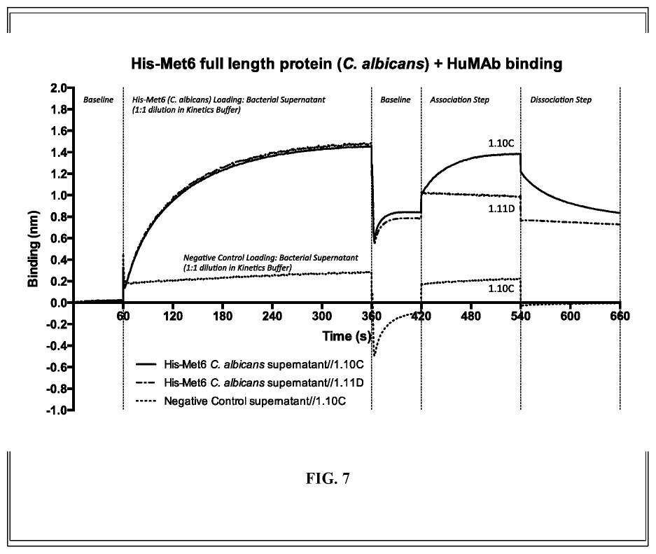

The results show that the human antibodies 1.10C (anti-MET6; SEQ ID NO: 12 and SEQ ID NO: 13 and 1.11D (anti-Fba; SEQ ID NO: 10 and SEQ ID NO: 11) specifically bind to the native recombinant MET6 and Fba proteins, respectively, from C. albicans (FIG. 6, top and FIG. 7) and that 1.11D (anti-Fba; SEQ ID NO: 10 and SEQ ID NO: 11) also binds to recombinant Fba from C. auris (FIG. 7) despite reduced homology of the C. auris peptide compared to the C. albicans peptide (Table 6). Furthermore, the results demonstrate that the Fba (Fba, SEQ ID NO: 40). and MET6 (MET6, SEQ ID NO: 38) peptide epitopes are accessible to antibody binding in the native proteins.

### Example 5

### Efficacy Assessment of Human Monoclonal Antibodies in the Mouse Lethal Model of Disseminated Candidiasis

### Materials and Methods

Candida Strains. C. albicans SC5314 (ATCC) and C. auris AR-0386 (CDC), which is an azole-resistant (Erg11 Y132F) South American strain, were grown as stationary-phase yeast cells in glucose-yeast extract-peptone broth at 37° C., washed and suspended to the appropriate cell concentration (C. albicans, 5×10⁶/ml; C. auris, 1×10⁹/ml) in Dulbecco's PBS (DPBS; Sigma), and used to infect mice intravenously (i.v.) as described (Han and Cutler, 1995; Han et al., 2000;

Mouse Strains. The inbred mouse strains C57BL/6 or A/J (NCI Animal Production Program or Harlan), (female; 5 to 7 weeks old) were used. Mice were maintained and handled in accordance with protocol approved by the Institutional Animal Care & Use committee (IACUC) regulations at Louisiana Health Sciences Center in New Orleans.

Fungal Challenge and Assessment of Protection. C57BL/6 mice or A/J), six to eight weeks old were used in these studies. Groups of three mice were housed together in sterile cages and provided sterile food and water ad libitum. On Day 0, groups of mice (one group per antibody) were injected i.p. by single injection of up to 0.5 ml of purified monoclonal antibodies (Prepared as above in Example 2) 4 hours prior to i.v. challenge with C. albicans 3153A cells (5×10⁵ CFU in 0.1 ml of DPBS) or C. auris (1×10⁸ CFU in 0.1 ml of DPBS). Protection was evaluated by monitoring animal survival for 35 days (C. albicans) or 40 days (C. auris). The mice were monitored for development of a moribund state, defined as being listless, disinterested in food or water, and nonreactive to finger probing. At the time that a mouse was deemed moribund, it was sacrificed. For comparison, one group received DPBS while another group received the antifungal drug Fuconazole™. Survival was assessed and compared to the controls.

### Results and Discussion

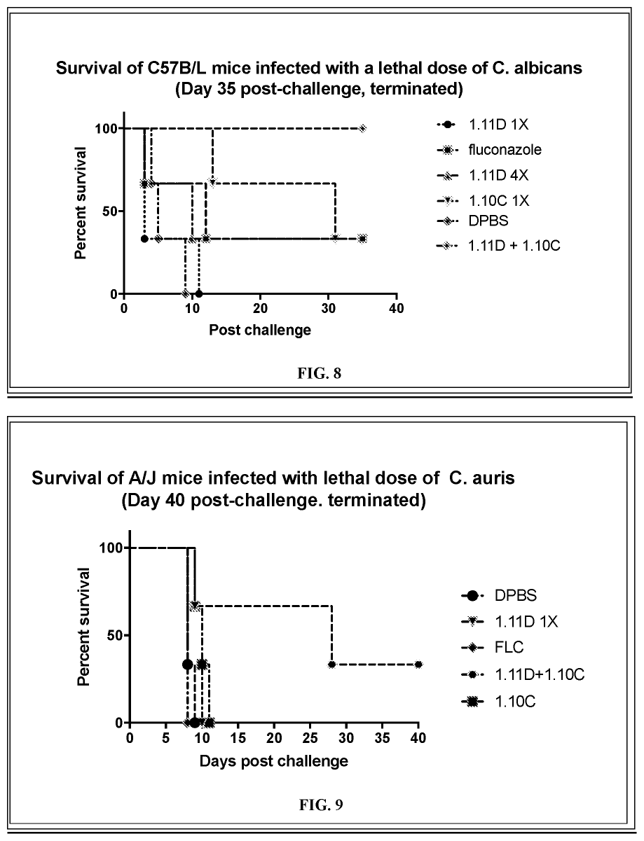

The results demonstrate that anti-peptide antibodies 1.10C and 1.11D, protect mice from death by C. albicans in the C57B/L6 mouse disseminated candidiasis model (FIG. 8), and a single dose of 1.10C provided better protection than the standard of care anti-fungal Fluconazole™. In addition, 1.11D demonstrated a clear dose response (FIG. 8), and a cocktail containing both antibodies provided complete protection. In the case of C. auris in the A/J neutropenic mouse disseminated candidiasis model, limited protection was observed using the individual antibodies alone, while a cocktail containing both antibodies enhanced protection of the mice (FIG. 9).

### Example 6

### Paratope Mapping for Met6

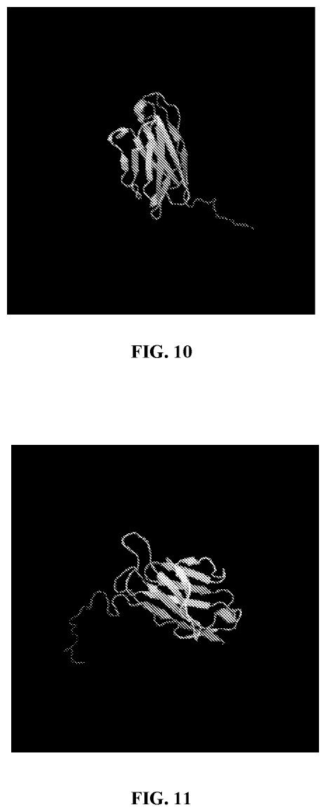

Protein modeling was conducted for Meth antibody 2B10 (FIGS. 10-11) according to The Phyre2 web portal for protein modeling, prediction and analysis by Kelley et al., Nature Protocols 10, 845-858 (2015).

2B10 VH (variable region heavy chain)
amino acid sequence:
(SEQ ID NO: 61)
MGWSYIILFLLATATRVHSQVQLQQPGAEVVRPGASVKVSCKASGYTVSS
YWMSWVKQRPEQGLEWIGRIDPYDSETRYNQKITKDKAILTVDKSSSTAY
MQLSSLTSEDSAVYYCARTAASFDYWGQGTTLTVSS

For 3D model of 2B10 VH (FIG. 10), the information can be retrieved at link:
https://nam01.safelinks.protection.outlook.com/?url=http%3A%2Fwww.sbg.bio.ic.ac.uk%2
Fphyre2%2Fphyre2_output%2Fcb0eb006b7305071%2Fsummary.html&amp;data=02%7C01%7
Chxin%40lsuhsc.edu%7Cda1cb96339c5434eb12408d70af98ac5%7C3406368982d44e89a3281a
b79cc58d9d%7C0%7C0%7C636989939222115355&amp;sdata=ZGX4T30tgM1IcNbAnWWBt
pkTqJkwiFldJiHeyfRKjEo%3D&amp;reserved=0

2B10 VL (variable region light chain)
amino acid sequence:
(SEQ ID NO: 62)
MKLPVRLLVMFWIPASSSDVVMTQTPLSLPVSLGDQASISCRSSQSLYHS
NGNSYLHWYLQKPGQSPKLLIYKVSNRFSGVPDRFSGSGSGTDFTLNISR
VEAEDLGVYFCSQSTHVPFTFGSGTKLEIK

For 3D model of 2B10 VL (FIG. 11), the information can be retrieved at link:
https://nam01.safelinks.protection.outlook.com/?url=http%3A%2F%2Fwww.sbg.bio.ic.ac.uk%2
Fphyre2%2Fphyre2_output%2Fd7ca058a43f777c0%2Fsummary.html&amp;data=02%7C01%7
Chxin%40lsuhsc.edu%7C9925559d95ac4b6cc5e008d70b04fb3b%7C3406368982d44e89a3281a
b79cc58d9d%7C0%7C0%7C636989988376463149&amp;sdata=Q%2Fbdj3r%2Fy2znuqy0Ru2
D1hWkqrKDBWeaiS9VpcZVPa0%3D&amp;reserved=0

### Paratome—Antigen Binding Regions Identification (ABR)

Mouse mAb 2B10C1 Specific for Met6 Peptide, Human Version of 2B1011C is 1.10C, 2B1011C V Sequences:

Heavy chain: DNA sequence (405 bp) (Leader
sequence-FR1-CDR1-FR2-CDR2-FR3-CDR3-FR4):
(SEQ ID NO: 63)
ATGGGATGGAGCTATATCATCCTCTTCTTGTTAGCAACAGCTACACGTGT
CCACTCCCAGGTCCAACTGCAGCAGCCTGGGGCTGAGGTGGTGAGGCCTG
GGGCTTCAGTGAAGGTGTCCTGCAAGGCTTCTGGCTACACGGTCAGCAGC
TACTGGATGAGCTGGGTTAAGCAGAGGCCGGAGCAAGGCCTTGAGTGGAT
TGGAAGGATTGATCCTTACGATAGTGAAACTCACTACAATCAAAAGTTCA
AGGACAAGGCCATATTGACTGTAGACAAATCCTCCAGCACAGCCTACATG
CAACTCAGCAGCCTGACATCTGAGGACTCTGCGGTCTATIACTGTGCAAG
GACGGCCGCTTCGTTTGACTATTGGGGCCAAGGCACCACTCTCACAGTCT
CCTCA
Heavy chain: Amino acids sequence (135 AA)
Leader sequence-FR1-CDR1-FR2-CDR2-FR3-
CDR3-FR4):
(SEQ ID NO: 61)
MGWSYIILFLLATATRVHSQVQLQQPGAEVVRPGASVKVSCKASGYTVSS
YWMSWVKQRPEQGLEWIGRIDPYDSETHYNQKFKDKAILTVDKSSSTAYM
QLSSLTSEDSAVYYCARtAASFDYWGQGTTLTVSS
Light chain: DNA sequence (393 bp) (Leader
sequence-FR1-CDR1-FR2-CDR2-FR3-CDR3-FR4):
(SEQ ID NO: 64)
ATGAAGTTGCCTGTTAGGCTGTTGGTGCTGATGTTCTGGATTCCTGCTTC
CAGCAGTGATGTTGTGATGACCCAAACTCCACTCTCCCTGCCTGTCAGTC
TTGGAGATCAAGCCTCCATCTCTTGCAGATCTAGTCAGAGCCTTGTACAC
AGTAATGGAAACTCCTATTTACATTGGTACCTGCAGAAGCCAGGCCAGTC
TCCAAAGCTCCTGATCTACAAAGTTTCCAACCGATTTTCTGGGGTCCCAG
ACAGGTTCAGTGGCAGTGGATCAGGGACAGATTTCACACTCAATATCAGC
AGAGTGGAGGCTGAGGATCTGGGAGTTTATTTCTGCTCTCAAAGTACACA
TGTTCCATTCACGTTCGGCTCGGGGACAAAGTTGGAAATAAAA
Light chain: Amino acids sequence (131 AA)
(Leader sequence-FR1-CDR1-FR2-CDR2-FR3-
CDR3-FR4):
(SEQ ID NO: 62)
MKLPVRLLVLMFWIPASSSDVVMTQTPLSLPVSLGDQASISCRSSQSLVH
SNGNSYLHWYLQKPGQSPKLLIYKVSNRFSGVPDRFSGSGSGTDFTLNIS
RVEAEDLGVYFCSQSTHVPFTFGSGTKLEIK
paratome_1_2910VH (heavy chain)
(SEQ ID NO: 65)
MGWSYIILFLLATATRVHSQVQLQQPGAEVVRPGASVKVSCKASGYTVSS
YWMSWVKQRPEQGLEWIGRIDPYDSETHYNQKFKDKAILTVKSSSTAYMQ
LSSLTSEDSAVYYCARTAASFDYWGQGTTLTVSS
ABR2: WIGRIDPYDSETHY (positions 66-79 of SEQ ID
NO: 65)
ABR3: ARTAASTDY (positions 115-123 of SEQ ID
NO: 65)
Legend: Heavy chain: ABR1 ABR2 ABR3
paratome_1_21310VL (light chain)
(SEQ ID NO: 62)
MKLPVRLLVLMFWIPASSSDVVMTQTPLSLPVSLGDQASISCRSSQSLVH
SNGNSVLHWYLQKPGQSPKLLIYKVSNRFSGVPDRFSGSGSGTDFTLNIS
RVEAEDLGVYFCSQSTHVPFTFGSGTKLEIK
ABR1: DVVMTQTPLSLPVSLGDQASISCRSSQSLVHSNGNSYLH
(positions 20-58 of SEQ ID NO: 62)
ABR2: KLLIYKVSNRFS (positions 69-80 of SEQ ID
NO: 62)
ABR3: SQSTHYPF (positions 113-120 of SEQ ID
NO: 62)
Legend: Light chain: ABR1 ABR2 ABR3

### Example 7

### Paratope Mapping for Fba

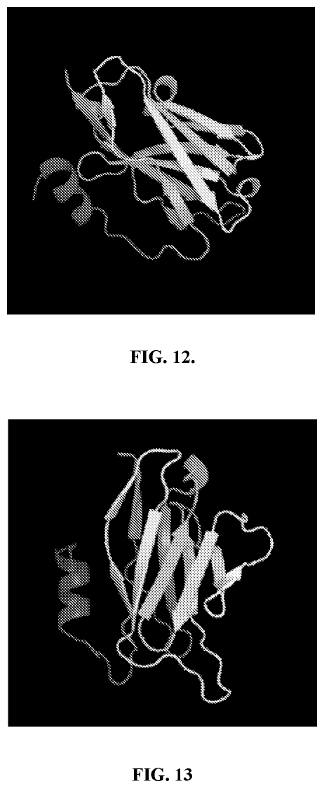

Protein modeling was conducted for Fba antibody 2B10 (FIGS. 12-13) according to The Phyre2 web portal for protein modeling, prediction and analysis by Kelley et al., Nature Protocols 10, 845-858 (2015).

2D5 VH (variable region heavy chain)
amino acid sequence:
(SEQ ID NO: 66)
MERHWIFLFLLSVTAGVHSQVQLQQSAAELARPGASVKMSCKASGYTFSS
YTMHWVKRPGQGLEWIGYINPSSGYTDYNQKFKDKTTLTADKSSSTAYMQ
LSSLTSEDSAVYYCRLYDNYDYYAMDYWGQGTSVTVSS

For 3D model of 2D5 VH (FIG. 12), the information can be retrieved at link:
https://nam01.safelinks.protection.outlook.com/?url=http%3A%2F%2Fwww.sbg.bio.ic.ac.uk%2
Fphyre2%2Fphyre2_output%2F51d51f35dfcbf7d6%2Fsummary.html&amp;data=02%7C01%7
Chxin%40lsuhsc.edu%7C88757e5de4324dd8eb8308d70b8ad368%7C3406368982d44e89a3281
ab79cc58d9d%7C0%7C0%7C636990563218376621&amp;sdata=YJ%2Fij5kU6KA7rYQVFiiA
%2FxvLmG8HkSGGSsRwsxbTVvw%3D&amp;reserved=0

2D5 VL (variable region light chain)
amino acid sequence:
(SEQ ID NO: 67)
M D S Q A Q V L I L L L L W V S G T C G D I V M S
Q S P S S L A V S A G E K V T M S C K S S Q S L L
N S R I R K N L A W Y Q Q K P G Q S P K L L I Y W
A S T R E S G V P D R F T G S G S G T D F T L T I
S S V Q A D D L A V Y Y C K Q Y N L L T F G A G T
K L E L K

For 3D model of 2D5 VL (FIG. 13), the information can be retrieved at link:
https://nam01.safelinks.protection.outlook.com/?url=http%3A%2F%2Fwww.sbg.bio.ic.ac.uk%2
Fphyre2%2Fphyre2_output%2F2c8a7c40d54a69f5%2Fsummary.html&amp;data=02%7C01%7
Chxin%40lsuhsc.edu%7C2b496735c49c4acf569b08d70b8b7f17%7C3406368982d44e89a3281a
b79cc58d9d%7C0%7C0%7C636990566093014306&amp;sdata=%2B%2B%2FBVCYeGv35A2
Urqvt7tDr%2BbvLEHbI9OfLYVZZwQuY%3D&amp;reserved=0

Paratome—Antigen Binding Regions Identification (ABR)

Mouse mAb 2D5F7 Specific for Fba Peptide, Human Version of 2D5F7 is 1.11D

Heavy chain: DNA sequence (420 bp) (Leader
sequence-FM-CDR1-FR2-CDR2-FR3-CDR3-FR4):
(SEQ ID NO: 68)
ATGGAAAGGCACTGGATCTTTCTCTTCCTGTTGTCAGTAACTGCAGGTGT
CCACTCCCAGGTCCAGCTGCAGCAGTCTGCAGCTGAACTGGCAAGACCTG
GGGCCTCAGTGAAGATGTCCTGCAAGGCTTCTGGCTACACCTTTAGTAGC
TACACGATGCACTGGGTAAAACAGAGGCCTGGACAGGGTCTGGAATGGAT
TGGATACATTAATCCTAGCAGTGGATATACTGATTACAATCAGAAGTTCA
AGGACAAGACCACATTGACTGCAGACAAATCCTCCAGCACAGCCTACATG
CAACTGAGCAGCCTGACATCTGAGGACTCTGCGGTCTATTACTGTGCAAG
ACTATATGATAACTACGATTACTATGCTATGGACTACTGGGGTCAAGGAA
CCTCAGTCACCGTCTCCTCA
Heavy chain: Amino acids sequence (140 AA)
(Leader sequence-FR1-CDR1-FR2-CDR2-FR3-
CDR3-FR4):
(SEQ ID NO: 69)
MERHWIFLFLLSVTAGVHSQVQLQQSAAELARPGASVKMSCKASGYTFSS
YTMHWVKQRPGQGLEWIGYINPSSGYTDYNQKFKDKTTLTADKSSSTAYM
QLSSLTSEDSAVYYCARLYDNYDYYAMDYWGQGTSVTVSS
Light chain: DNA sequence (396 bp) (Leader
sequence-FR1-CDR1-FR2-CDR2-FR3-CDR3-FR4):
(SEQ ID NO: 70)
ATGGATTCACAGGCCCAGGTTCTTATATTGCTGCTATGGGTATCTGGTAC
CTGTGGGGACATTGTGATGTCACAGTCFCCATCCTCCCTGGCTGTGTCAG
CAGGAGAGAAGGTCACTATGAGCTGCAAATCCAGTCAGAGTCTGCTCAAT
AGTAGAATCCGAAAGAACTACTTGGCTTGGTACCAGCAGAAACCAGGGCA
GTCTCCTAAACTGCTGATCTACTGGGCATCCACTAGGGAATCTGGGGTCC
CTGATCGCTTCACAGGCAGTGGATCTGGGACAGATTTCACTCTCACCATC
AGCAGTGTGCAGGCTGATGACCTGGCAGTTTATTACTGCAAGCAATCTTA
TAATCTGCTCACGTTCGGTGCTGGGACCAAGCTGGAGCTGAAA
Light chain: Amino acids sequence (132 AA)
(Leader sequence-FR1-CDR1-FR2-CDR2-FR3-
CDR3-FR4):
(SEQ ID NO: 71)
MDSQAQVLILLLLWVSGTCGDIVMSQSPSSLAVSAGEKVTMSCKSSQSLL
NSRIRKNYLAWYQQKPGQSPKLLIYWASTRESGVPDRFTGSGSGTDFTLT
ISSVQADDLAVYYCKQSYNLLTFGAGTKLELK
paratome 1_2D5_VH (heavy chain)
(SEQ ID NO: 72)
MERHWIFLFLLSVTAGVHSQVQLQQSAAELARPGASVKMSCKASGYTFSS
YTMHWVKQRPGQGLEWIGYINPSSGYTDYNQKFKDKTTLTAKSSSTAYMQ
LSSLTSEDSAVYYCARLYDNYDYYAMDYWGQGTSVTVSS
ABR2: WIGYINPSSGYTDY (positions 66-79 of SEQ
ID NO: 72)
ABR3: RLYDNYDYYAMDY (positions 116-128 of SEQ
ID NO: 72)
Legend: Heavy chain: ABR1 ABR2 ABR3
paratome_1_2D5VL (light chain)
(SEQ ID NO: 71)
MDSQAQVLILLLLWVSGTCGDIVMSQSPSSLAVSAGEKVTMSCKSSQSLL
NSRIRKNYLAWYQQKPGQSPKLLIYWASTRESGVPDRFTGSGSGTDFTLT
ISSVQADDLAVYYCKQSYNLLTFGAGTKLELK
ABR1: GDIVMSQSPSSLAVSAGEKVTMSCKSSQSLLNSRIRKNYLA
(20-60)
ABR2: LLIYWASTRES (positions 72-82 of SEQ ID
NO: 71)
ABR3: KQSYNLL (positions 115-121 of SEQ ID
NO: 71)
Legend: Light chain: ABR1 ABR2 ABR3

### Example 8

### Antibody Binding Kinetics

Binding experiments were performed using a FortèBio BLITz Bi-layer interferometer at 25° C. Streptavidin (SA) biosensors were hydrated in Assay Buffer (PBS with 0.1% BSA, 0.02% Tween-20 (pH 7.4)), and biotinylated peptides (Fba-Biotin or Met6-Biotin) at 0.01 μg/mL in Assay Buffer were loaded onto Streptavidin (SA) biosensors. Loaded sensors were dipped into serial dilutions of the cognate IgG.

For antibody production, matched pairs of plasmids expressing the heavy and light chains of either 1.10C (anti-Met6) or 1.11D (anti-Fba) were transiently transfected into Freestyle™ 293 cells and secreted IgG was purified by Fast Flow Protein G (GE Life Sciences) affinity chromatography.

Binding experiments were performed using a FortèBio BLITz Bi-layer interferometer at 25° C. Streptavidin (SA) biosensors were hydrated in Assay Buffer (PBS with 0.1% BSA, 0.02% Tween-20 (pH 7.4)), and biotinylated peptides (Fba-Biotin or Met6-Biotin) at 0.01 μg/mL in Assay Buffer were loaded onto Streptavidin (SA) biosensors. Loaded sensors were dipped into serial dilutions of the cognate IgG.

For antibody production, matched pairs of plasmids expressing the heavy and light chains of either the human anti-Met6 or anti-Fba antibodies were transiently transfected into Freestyleä 293 cells and secreted IgG was purified by Fast Flow Protein G (GE Life Sciences) affinity chromatography. The results shown in Table 8 demonstrate that the antibodies bind to their cognate peptide with binding affinities (kD) at 1×10⁷ or better.

TABLE 1
NUCLEOTIDE SEQUENCES FOR ANTIBODY VARIABLE REGIONS

| | | SEQ |
|---|---|---|
| Clone | Variable Sequence Region | ID NO: |
| 1.11D | GAAGTGCAGCTGGTGCAGTCTGGGGGAGGCTTGGTCCAGCCTGGGGGGTCC | 1 |
| heavy | CTGAGACTCTCTTGTTCAGCCTCTGGGTTCACCTTTAGAACCTATGCCATG | |
| | AGCTGGGTCCGCCAGGCTCCAGGGAAGGGGCTGCAGTGGGTCTCAGTTATT | |
| | AGTCGTAGTGGTGATACCACCTACCACACAGACTCCGTGAAGGGCCGATTC | |
| | ACCATCTCCAGAGACAATTCCAGGAACGCGCTGTATCTGCAATTGGACAGC | |
| | CTGAGAGCCGAGGACACGGCCTTATATTACTGTGCGAAAACAGGTAATATG | |
| | GCAGTAGGTGACCGAAGGACAAACTACTCCTACTACTACATGGACGTCTGG | |
| | GGCAAAGGGACCACGGTCACCGTCTCCTCA | |
| 1.11D | GATATTGTGATGACTCAGTCTCCTTCCACCCTGTCTGCTTCTGTAGGAGAC | 2 |
| light | AGAGTCACCATCACTTGCCGGGCCAGTCAGAGTATTAAGTACTGGTTGGCC | |
| | TGGTATCAGCAGAAACCAGGGAAAGCCCCTAAGCTCCTGATCTATAAGGCA | |
| | TCTAATTTGGAAAGTGGGGTCCCATCAAGGTTCAGCGGCAGTGGATCTGGG | |
| | ACAGAATTCACTCTCACCATCAGCAGCCTGCGGCCTGATGATTTTGCAACT | |
| | TATTACTGCCAACAGTATAATAGTTACCCCCTCACTTTCGGCGGAGGGACC | |
| | ACGGTGGAGATCAAA | |
| 1.10C | GAGGTGCAGCTGGTGGAGTCTGGGGGAGGCTTGGTAAAGCCTGGGGGGTCC | 3 |
| heavy | CTGAGACTCTCCTGTAAAGCATCTGGATTCAATTTCACTAACTCCTGGATG | |
| | AGTTGGGTCCGCCAGGCTCCAGGGAAGGGACTGGAGTGGCTGGGTCGTATT | |
| | AAAAGTGAGTCTGATGGTGGGGCAACACGCTACGCTGCACCCGTTACGGGA | |
| | AGGTTTTCCATCTCCAGAGATGATTCAAGAGACATGCTGTTTCTGCAAATG | |
| | AACAGTCTGACAACCGACGACACAGCGATGTATTATTGTACTACAAATAAG | |
| | GTGACTACAAATTATTGGGGCCAGGGAACGCTGGTCACCGTCTCATCA | |
| 1.10C | GACATTGTGATGACTCAGTCTCCAGTCACCCTGGCTGTGTCTCTGGGCGAG | 4 |
| light | AGGGCCACCATCAACTGCAAGTCCAGCCAGAGTCTTTTATACAGCTCCGAC | |
| | AATGAGAACTACTTAACTTGGTACCAGCAGAAACCAGGACAGCCTCCTAAG | |
| | TTGCTCATTTACTGGGCGTCTGTCCGAGAATCCGGGATTCCTGACCGATTC | |
| | ATTGGCAGCGGGTCTGTGACAGATTTCACTCTCACCATCAACAATGTGCAG | |
| | GCTGAAGATGTGGCAGTTTATTACTGTCAACAATTTCGCTATACTCCTCTG | |
| | ACTTTTGGCCAGGGGACCACGCTTGAGATCAAA | |
| 1.14M | GAGGTTCAGCTGGTGGAGTCTGGGGCTGAGGTGAAGAGGCCTGGGGCCTCA | 5 |
| heavy | GTGAGGGTCTCCTGCAAGGCTTCTGGATACAGCTTCACCCTCTACTATATG | |
| | CACTGGGTGCGACAGGCCCCTGGCCAAGGACTCGAGTGGCTGGGATGGATC | |
| | AACCCTAAAACTGGTGACGTCAAATATGCACAGAAGTTTCAGGGCAGGGTC | |
| | TCCTTGACCAGGGATACGAGAATGAACACAGCCTACTTGGACTTGACGAGG | |
| | CTGAGATCTGACGACACGGCCCGCTACTACTGTTTGAGGGCTTTTGATCTG | |
| | TGGGGCCGAGGGACAATGATCATCGTCTCCTCA | |
| 1.14M | CTGCCTGTGCTGACTCAGCCACCCTCGGTGTCAGTETCCCCAGGACAAACG | 6 |
| light | GCCAGGATCACCTGCTGGAGATACATTGGCAAAGAAATATGCTTATTGGTA | |
| | CCAGCAGAAGTCAGGCCAGGCCCCTGTTCTGGTCATCCAAGACGACACCAA | |
| | GCGACCCTCCGGGATCCCTCAGCGATTCTCTGGCTCAAGCTCAGGGACAAT | |
| | GGCCACCTTGACTATAAGTGCGGCCCAGGTGGAGGATGAAGCTGACTACCA | |
| | CTGCTTCTCAACAGATGATAGTGGAAATCCTGAGGGCCTCTTCGGCGGAGG | |
| | AACCAAACTGACCGTCCTAAGTCAGCCCAAGGCTGCCCCCTCGGTCACTCT | |
| | G | |
| 6.6K | CAGGTGCAGCTGGTGGAGTCTGGGGGAGGCGTGGTCCAGCCTGGGAGGTCC | 7 |
| heavy | CTGAGACTCTCCTGTGCAGCCTCTGGATTCAACTTCATTAGTTATGGCATG | |
| | CACTGGGTCCGCCAGGCTCCAGGCAAGGGGCTGGAGTGGGTGGCACTTATT | |
| | TCATATGATGGAAGTAATAAATACTATGCAGACTCCGTGAAGGGCCGATTC | |
| | ACCATCTCCAGAGACAATTCCAAGAACACGCTGTATCTGCAAATGAACAGC | |
| | CTGAGAGCTGAGGACACGGCTGTATATTACTGTGCGACCGAGGCTTACGTG | |
| | GAAACAGCTATGGTCCCCCAGTACTGGGGCCAGGGAACCCTGGTCACCGTC | |
| | TCCTCA | |
| 6.6K | TCTTATGAGCTGACTCAGCCACCCTCGGTGTCAGTGTCCCCAGGACAAACG | 8 |
| light | GCCAGGATCACCTGCTCTGGAGATGCATTGCCAAAAGAATATGCTTATTGG | |
| | TACCAGCAGAAGTCAGGCCAGGCCCCTGTGGTGGTCATCTATGAAGACAGC | |
| | AAACGACCCTCCGGGATCCCTGAGCGATTCTCTGGCTCCAGCTCAGGGACA | |
| | ATGGCCACCTTGACTATCAGTGGGGCCCAGGTGGAGGATGAAGCTGACTAC | |
| | CACTGTTACTCAACAGACAGCAGTGGTAATCCCGTGTTCGGCGGAGGGACC | |
| | AAGCTGACCGTCCTA | |
| 1.B10 | GAGGTGCAGCTGGTGCAGTCTGGAGGAGGCTTGGTAAAGCCTGGGGGGTCC | 42 |
| heavy | CTTAGACTTTCCTGTGCAGCCTCTGGATTCATTTTCAGTAACGCCTGGATG | |
| | AACTGGGTCCGCCAGGCTCCAGGGAAGGGGCTGGAGTGGGTTGGCCGTATT | |
| | AAAAGAGAAAGTGATAGTGGGACAACAGACTACGGTGCAGCCGTGAAAGGC | |
| | AGATTCACCATCTCAAGAGATGATTCAAAATACACGCTGTATCTGCAAATG | |
| | AACAGCCTGAAAACCGACGACACAGCCETTTATTACTGTACCACAGGGTGG | |
| | GCTGACTACTGGGGCCAGGGAACCCTGGTCACCGTCTCCTCA | |
| 1.B10 | CTGATTCAGCCACCCTCGGTGTCAGTGTCCCCAGGACAGACGGCCAGGATC | 43 |
| light | ACCTGCTCTGGAGATGCATTGCCAAACAAATATGCTTATTGGTACCAGCAG | |
| | AAGCCAGGCCAGGCCCCTTCTGTGGTGATGTTTAGAGACAATGAGAGACCC | |
| | TCAGGGATCCCTGAGCGATTCTCTGGCTCCAGCTCAGGGACAACAGTCACG | |
| | TTGACCATCAGTGGAGTCCAGGCAGAAGACGAGTCTGACTTTTATTGTCAA | |
| | TCCACAGACAGTAATGGTGCTTGGGTGTTCGGCGGAGGGACCAAGCTGACC | |
| | GTCCTA | |
| 6.11C | CAGGTGCAGTTGGTGCAGTCTGGGGCTGAGGTGAAGAAGCCTGGGGCCTCA | 44 |
| Heavy | GTTCAAGTTTCCTGCAGGACATCTGGATACACCTTTATTAATTATTTTATG | |
| | CACTGGGTGCGACAGGCCCCTGGGCAAGGGCTTGAGTGGATGGGAATAATC | |
| | AACCCTAATGGTGGTAAGACAAGATACGCACAGAAGTTCCAGGGCAGACTC | |
| | ACCGTGACCAGGGACACGTCCACCAACACTGTCTACGTGGAACTGAGCAAT | |
| | CTGAGATATGAGGACACGGGCCTCTATTTCTGCGCGAGAGATCCGGAGGGG | |
| | GAAGTGGGCTTTGACTACTGGGGCCAGGGAACCCAGGTCACCGTCTCCTCA | |
| 6.11C | TCCCATGAACTGACACAGCCACCCTCGGTGTCAGTGTCCCCAGGACAGACG | 45 |
| light | GCCAGGATCACCTGCTCTGGAGATGCACTGTCAAAGCAATATGCTTATTGG | |
| | TATCAGCAGAAGCCAGGCCAGGCCCCTGTGGTGGTGATATATAAAGACAAT | |
| | GAGAGGCCCTCAGGGATCCCTGAGCGATTCTCTGGCTCCAGTTCAGGCACA | |
| | ACAGTCACATTGACCATCACTGGAGTCCAGGCAGAAGACGAGGCTGACTAT | |
| | TATTGTCAATCAACAGACACCAGTCGTGCTTATTATGTCTTCGGAACTGGG | |
| | ACCAAGGTCACCGTCTTA | |

TABLE 2
PROTEIN SEQUENCES FOR ANTIBODY VARIABLE REGIONS

| | | SEQ |
|---|---|---|
| | | ID |
| Clone | Variable Sequence | NO. |
| 1.11D | EVQLVQSGGGLVQPGGSLRLSCSASGFTFRTYAMSWVRQAPG | 10 |
| heavy | KGLQWVSVISRSGDTTYFITDSVKGRFTISRDNSRNALYLQL | |
| | DSLRAEDTALYYCAKTGNMAVGDRRTNYSYYYMDVWGKGTTV | |
| | TVSS | |
| 1.11D | DIVMTQSPSTLSASVGDRVTTTCRASQSTKYWLAWYQQKPGK | 11 |
| light | APKLLIYKASNLESGVPSRFSGSGSGTEFTLTISSLRPDDFA | |
| | TYYCQQYNSYPLTFGGGTVEIK | |
| 1.10C | EVQLVESGGGLVKPGGSLRLSCKASGFNFTNSWMSWVRQAPG | 12 |
| heavy | KGLEWLGRIKSESDGGATRYAAPVTGRFSISRDDSRDMLFLQ | |
| | MNSLTTDDTAMYYCTTNKVTTNYWGQGTLVTVSS | |
| 1.10C | DIVMTQSPVTLAVSLGERATINCKSSQSLLYSSDNENYLTWY | 13 |
| light | QQKPGQPPKLLIYWASVRESGIPDRFIGSGSVTDFTLTINNV | |
| | QAEDVAVYYCQQFRYTPLTFGQGTTLEIK | |
| 1.14M | EVQLVESGAEVKRPGASVRVSCKASGYSFTLYYMHWVRQAPG | 14 |
| heavy | QGLEWLGWINPKTGDVKYAQKFQGRVSLTRDTRMNTAYLDLT | |
| | RLRSDDTARYYCLRAFDLWGRGTMIIVSS | |
| 1.14M | LPVLTQPPSVSVSPGQTARITCSGDTLAKKYAYWYQQKSGQA | 15 |
| light | PVLVIQDDTKRPSGIPQRFSGSSSGTMATLTISAAQVEDEAD | |
| | YHCFSTDDSGNPEGLFGGGTKLTVLSQPKAAPSVTL | |
| 6.6K | QVQLVESGGGVVQPGRSLRLSCAASGFNFISYGMHWVRQAPG | 16 |
| heavy | KGLEWVALISYDGSNKYYADSVKGRFTISRDNSKNTLYLQMN | |
| | SLRAEDTAVYYCATEAYVETAMVPQYWGQGTLVTVSS | |
| 6.6K | SYELTQPPSVSVSPGQTARITCSGDALPKEYAYWYQQKSGQA | 17 |
| light | PVVVIYEDSKRPSGIPERFSGSSSGTMATLTISGAQVEDEAD | |
| | YHCYSTDSSGNPWGGGTKLTVL | |
| 1.B10 | EVQLVQSGGGLVKPGGSLRLSCAASGFIFSNAWMNWVRQAPG | 46 |
| heavy | KGLEWVGRIKRESDSGTTDYGAAVKGRFTISRDDSKYTLYLQ | |
| | MNSLKTDDTAVYYCTTGWADYWGQGTLVTVSS | |
| 1B10 | LIQPPSVSVSPGQTARITCSGDALPNKYAYWYQQKPGQAPSV | 47 |
| light | VMFRDNERPSGIPERFSGSSSGTTVTLTISGVQAEDESDFYC | |
| | QSTDSNGAWVFGGGTKLTVL | |
| 611C | QVQLVQSGAEVKKPGASVQVSCRTSGYTFINYFMHWVRQAPG | 48 |
| heavy | QGLEWMGIINPNGGKTRYAQKFQGRLTVTRDTSTNTVYVELS | |
| | NLRYEDTGLYFCARDPEGEVGFDYWGQGTQVTVSS | |
| 611C | SHELTQPPSVSVSPGQTARITCSGDALSKQYAYWYQQKPGQA | 49 |
| light | PVVVIYKDNERPSGIPERFSGSSSGTTVTLTITGVQAEDEAD | |
| | YYCQSTDTSRAYYVFGTGTKVTVL | |

TABLE 3
CDR HEAVY CHAIN SEQUENCES

| | CDRH1 | CDRH2 | CDRH3 |
|---|---|---|---|
| | (SEQ ID | (SEQ ID | (SEQ ID |
| Antibody | NO:) | NO:) | NO:) |
| 1.11D | GFTFRTYA | ISRSGDTT | AKTGNMAVG |
| | (18) | (19) | DRRT |
| | | | (20) |
| 1.10C | GFNFTNSW | IKSESDGGAT | TTNKVTTNY |
| | (21) | (22) | (23) |
| 1.14M | GYSFFINY | INPKTGDV | LRAFDL |
| | (24) | (25) | (26) |
| 6.6K | GFNHSYG | ISYDGSNK | ATEAYVETA |
| | (27) | (28) | MVPQY |
| | | | (29) |
| 1.B10 | GFIFSNAW | IKRESDSGTT | TTGWADY |
| | (50) | (51) | (52) |
| 6.11C | NYFMH | IINPNGGKTR | DPEGEVGFD |
| | (53) | YAQKFQG | Y (55) |
| | | (54) | |

TABLE 4
CDR LIGHT CHAIN SEQUENCES

| | CDRH1 | CDRH2 | CDRH3 |
|---|---|---|---|
| | (SEQ ID | (SEQ ID | (SEQ ID |
| Antibody | NO:) | NO:) | NO:) |
| 1.11D | QSIKYW | KAS | QQYNSYPLI |
| | (30) | | (31) |
| 1.10C | QSLLYS | WAS | QQFRYTPLT |
| | SDNENY | | (33) |
| | (32) | | |
| 1.14M | TLAKKY | DDT | FSTDDSGNP |
| | (34) | | EGL |
| | | | (35) |
| 6.6K | ALPKEY | EDS | YSTDSSGNP |
| | (36) | | V (37) |
| 1B10 | ALPNKY | RDN | QSTDSNGAW |
| | (56) | | V (57) |
| 6.11C | SGDALS | KDNERPS | QSTDTSRAY |
| | KQYAY | (59) | YV (60) |
| | (58) | | |

TABLE 5
PEPTIDE SEQUENCES

| | PEPTIDE |
|---|---|
| Name | (SEQ ID NO:) |
| MEM | PRIGGQRELKKITE |
| | (38) |
| MET6- | PRIGGQRELKKITE |
| Biotin | PGGSGGSGK- |
| | Biotin (39) |
| Fba | YGKDVKDLFDYAQE |
| | (40) |
| Fba- | YGKDVKDLFDYAQE |
| Biotin | GGSGGSGK- |
| | Biotin (41) |

TABLE 6
ANTIBODY/PEPTIDE DESIGNATIONS

| | HEAVY | | |
|---|---|---|---|
| | CHAIN/ | | |
| | LIGHT | | |
| | CHAIN | | |
| | (SEQ ID | | PEPTIDE |
| Clone | NO:) | Name | (SEQ ID NO:) |
| 1.10C | (12/13) | MET6 | PRIGGQRELKKITE |
| | | | (38) |
| 6.6K | (16/17) | MET6 | PRIGGQRELKKITE |
| | | | (38) |
| 1.B10 | (46/47) | MET6 | PRIGGQRELKKITE |
| | | | (38) |
| 1.11D | (10/11) | Fba | YGKDVKDLFDYAQE |
| | | | (40) |
| 1.14M | (14/15) | Fba | YGKDVKDLFDYAQE |
| | | | (40) |
| 6.11C | (48/49) | Fba | YGKDVKDLFDYAQE |
| | | | (40) |

TABLE 7
HOMOLOGY OF THE FBA and MET6 PEPTIDE SEQUENCES
BETWEEN CANDIDA SPECIES THAT ARE HUMAN PATHOGENS

| | Fba PEPTIDE | MET6 PEPTIDE |
|---|---|---|
| Candida spp. | (SEQ ID 40:) | (SEQ ID 38:) |
| Candida albicans | 100% | 100% |
| Candia glabrata | Not available | 85% |
| Candida | 100% | 100% |
| parapsilosis | | |
| Candia tropicalis | 91% | 100% |
| Candida dubliniensis | 100% | 100% |
| Candida krusei | 100% | 100% |
| Candida auris | 85% | 79% |

TABLE 8
BLITz Kinetics Data Summary for HuMAb Candida Antibodies (Autoimmune Technologies)

| # | Date of run | ID code | Ab Type | KD (M) | ka (1/Ms) | kd (1/s) | BLITz Tip | Antigen loaded | Comments Specificity |
|---|---|---|---|---|---|---|---|---|---|
| 1 | Nov. 28, 2018 | 1.11D | HuMAb | 1.98E−08 | 3.80E+04 | 7.30E−04 | Strep (SA) | btn-Fba peptide | Fba |
| 2 | Dec. 3, 2018 | 1.10C | HuMAb | 1.80E−07 | 3.10E+04 | 5.50E−03 | Strep (SA) | btn-Met6 peptide | Met6 |
| 3 | Oct. 2, 2019 | 1.B10 | HuMAb | 3.10E−08 | 2.10E+05 | 6.40E−03 | Strep (SA) | btn-Met6 peptide | Met6 |
| 4 | Oct. 10, 2019 | 6.6K | HuMAb | 1.60E−07 | 7.50E+04 | 1.20E−02 | Strep (SA) | btn-Met6 peptide | Met6 |
| 5 | Jun. 18, 2020 | 6.11C | HuMAb | 1.20E−07 | 4.90E+05 | 6.00E−02 | Strep (SA) | btn-Fba peptide | Fba |

All of the compositions and methods disclosed and claimed herein can be made and executed without undue experimentation in light of the present invention. While the compositions and methods of this disclosure have been described in terms of preferred embodiments, it will be apparent to those of skill in the art that variations can be applied to the compositions and methods and in the steps or in the sequence of steps of the method described herein without departing from the concept, spirit and scope of the disclosure. More specifically, it will be apparent that certain agents which are both chemically and physiologically related can be substituted for the agents described herein while the same or similar results would be achieved. All such similar substitutes and modifications apparent to those skilled in the art are deemed to be within the spirit, scope and concept of the disclosure as shown by the appended claims.

### VII. REFERENCES

The following references, to the extent that they provide exemplary procedural or other details supplementary to those set forth herein, are specifically incorporated herein by reference.
U.S. Pat. No. 3,817,837
U.S. Pat. No. 3,850,752
U.S. Pat. No. 3,939,350
U.S. Pat. No. 3,996,345
U.S. Pat. No. 4,196,265
U.S. Pat. No. 4,275,149
U.S. Pat. No. 4,277,437
U.S. Pat. No. 4,366,241
U.S. Pat. No. 4,472,509
U.S. Pat. No. 4,554,101
U.S. Pat. No. 4,680,338
U.S. Pat. No. 4,816,567
U.S. Pat. No. 4,867,973
U.S. Pat. No. 4,938,948
U.S. Pat. No. 5,021,236
U.S. Pat. No. 5,141,648
U.S. Pat. No. 5,196,066
U.S. Pat. No. 5,563,250
U.S. Pat. No. 5,565,332
U.S. Pat. No. 5,856,456
U.S. Pat. No. 5,880,270
U.S. Pat. No. 6,485,982
“Antibodies: A Laboratory Manual,” Cold Spring Harbor Press, Cold Spring Harbor, N.Y., 1988.
Abbondanzo et al., Am. J. Pediatr. Hematol. Oncol., 12(4), 480-489, 1990.
Allred et al., Arch. Surg., 125(1), 107-113, 1990.
Atherton et al., Biol. of Reproduction, 32, 155-171, 1985.
Barzon et al., Euro Surveill. 2016 Aug. 11; 21(32).
Beltramello et al., Cell Host Microbe 8, 271-283, 2010.
Brown et al., J. Immunol. Meth., 12; 130(1):111-121, 1990.
Campbell, In: Monoclonal Antibody Technology, Laboratory Techniques in Biochemistry and Molecular Biology, Vol. 13, Burden and Von Knippenberg, Eds. pp. 75-83, Amsterdam, Elsevier, 1984.
Capaldi et al., Biochem. Biophys. Res. Comm., 74(2):425-433, 1977.
Casadevall et al., Cur. Opin. Micro. 5:386-391, 2002.
Chowdry et al., Emerg. Infect. Dis., 19:1670-1673, 2002
Costin et al., J. Vorol. 71:5069-5079, 2013.
De Jager et al., Semin. Nucl. Med. 23(2), 165-179, 1993.
Dholakia et al., J. Biol. Chem., 264, 20638-20642, 1989.
Diamond et al., J Virol 77, 2578-2586, 2003.
Doolittle and Ben-Zeev, Methods Mol. Biol., 109:215-237, 1999.
Duffy et al., N Engl. J. Med. 360, 2536-2543, 2009.
Elder et al. Infections, infertility and assisted reproduction. Part II: Infections in reproductive medicine & Part III: infections and the assisted reproductive laboratory, Cambridge UK: Cambridge University Press; 2005.
Gancedo et al., Micro. Mol. Bio. Rev. 80:765-777, 2016.
Gefter et al., Somatic Cell Genet., 3:231-236, 1977.
Gornet et al., Semin Reprod Med. 2016 September; 34(5):285-292. Epub 2016 Sep. 14.
Gulbis and Galand, Hum. Pathol. 24(12), 1271-1285, 1993.
Halfon et al., PLoS ONE 2010; 5 (5) e10569
Han and Cutler, Infet. Immun. 63:2714-2719, 1995
Han et al., Onfet. Immun. 68:1649-1654, 2000.
Henderson and Martin, Infect. Immun., 79:3476-3491, 2011.
Hessell et al., Nature 449, 101-4, 2007.
Karwa and Wargo, Ann. Pharmacother., 43:1818-1823, 2009
Khatoon et al., Ann. of Neurology, 26, 210-219, 1989.
Kim and Sudbery, J. Microbiol., 49:171-177, 2011
King et al., J. Biol. Chem., 269, 10210-10218, 1989.
Kohler and Milstein, Eur. J. Immunol., 6, 511-519, 1976.
Kohler and Milstein, Nature, 256, 495-497, 1975.
Kyte and Doolittle, J. Mol. Biol., 157(1):105-132, 1982.
Li et al., BMC Infect. Diseas., 13(1):253-261, 2013.
Low and Rotstein, F1000 Medicine Reports, 3(14), 2011.
Luo et al., Blood, 113:1422-1431, 2009.
Luo, et al., J. Immunol. 185:2164-2173, 2010.
Mansuy et al., Lancet Infect Dis. 2016 October; 16(10):1106-7.
Medrano-Diaz, et al., Microbiol. Pathogen., 124:21-29, 2018.
Nakamura et al., In: Enzyme Immunoassays: Heterogeneous and Homogeneous Systems, Chapter 27, 1987.
O'Shannessy et al., J. Immun. Meth., 99, 153-161, 1987.
Persic et al., Gene 187:1, 1997.
Potter and Haley, Meth. Enzymol., 91, 613-633, 1983.
Purpura et al., Lancet Infect Dis. 2016 October; 16(10):1107-8. Epub 2016 Sep. 19.
Remington's Pharmaceutical Sciences, 15th Ed., 3:624-652, 1990.
Richie et al., Antimirob. Agents Chemo., 56:3963-3964, 2012.
Robinson et al., Nat. Commun. 7:11544, 2016 May 10.
Rudkin et al., Nat. Commun. 9:5288, 2018.
Tang et al., J. Biol. Chem., 271:28324-28330, 1996.
Tiller et al., Immunol. Methods 329:112-124, 2008.
Turner and Butler, Cold Spring Harb. Perspect. Med., 4(9), 2014.
Wawyrzynczak & Thorpe, In: Immunoconjugates, Antibody Conuugates In Radioimaging And Therapy Of Cancer, Vogel (Ed.), NY, Oxford University Press, 28, 1987.
Xin et al., Proc. Natl. Acad. Sci. USA. 105:13526-1353, 2008.
Yu et al., J Immunol Methods 336, 142-151, doi:10.1016/j.jim.2008.04.008, 2008.

1. A method of detecting a Candida infection in a subject comprising:
(a) contacting a sample from said subject with an antibody or antibody fragment, wherein the antibody or antibody fragment comprises clone-paired heavy and light chain CDR sequences, wherein the heavy chain CDR sequences are selected from Table 3, and the light chain CDR sequences are selected from Table 4; and
(b) detecting Candida in said sample by binding of said antibody or antibody fragment to a Candida antigen in said sample.

2. The method of claim 1, wherein said sample is a body fluid.

3. The method of claim 1, wherein said sample is blood, sputum, tears, saliva, mucous or serum, semen, cervical or vaginal secretions, amniotic fluid, placental tissues, urine, exudate, transudate, tissue scrapings or feces.

4. The method of claim 1, wherein detection comprises ELISA, RIA, lateral flow assay or Western blot.

5. The method of claim 1, further comprising performing steps (a) and (b) a second time and determining a change in Candida antigen levels as compared to the first assay.

6. The method of claim 1, wherein said antibody or antibody fragment is encoded by light and heavy chain variable nucleotide sequences according to clone-paired sequences selected from Table 1.

7. The method of claim 1, wherein said antibody or antibody fragment is encoded by light and heavy chain variable nucleotide sequences having at least 70%, 80%, or 90% identity to clone-paired sequences selected from Table 1.

8. The method of claim 1, wherein said antibody or antibody fragment is encoded by light and heavy chain variable nucleotide sequences having at least 95% identity to clone-paired sequences selected from Table 1.

9. The method of claim 1, wherein said antibody or antibody fragment comprises a light chain variable sequence and a heavy chain variable sequence selected from clone-paired sequences of Table 2.

10. The method of claim 1, wherein said antibody or antibody fragment comprises light and heavy chain variable sequences having 70%, 80% or 90% identity to clone-paired sequences from Table 2.

11. The method of claim 1, wherein said antibody or antibody fragment comprises light and heavy chain variable sequences having 95% identity to clone-paired sequences from Table 2.

12. The method of claim 1, wherein the antibody fragment is a recombinant scFv (single chain fragment variable) antibody, Fab fragment, F(ab′)₂ fragment, or Fv fragment.

13. A method of treating a subject infected with Candida or reducing the likelihood of infection of a subject at risk of contracting Candida comprising delivering to said subject an antibody or antibody fragment, wherein the antibody or antibody fragment comprises clone-paired heavy and light chain CDR sequences, wherein the heavy chain CDR sequences are selected from Table 3, and the light chain CDR sequences are selected from Table 4.

14. The method of claim 13, wherein said antibody or antibody fragment is encoded by light and heavy chain variable nucleotide sequences according to clone-paired sequences selected from Table 1.

15. The method of claim 13, wherein said antibody or antibody fragment is encoded by light and heavy chain variable nucleotide sequences having at least 70%, 80%, or 90% identity to clone-paired sequences selected from Table 1.

16. The method of claim 13, wherein said antibody or antibody fragment is encoded by light and heavy chain variable nucleotide sequences having at least 95% identity to clone-paired sequences selected from Table 1.

17. The method of claim 13, wherein said antibody or antibody fragment comprises a light chain variable sequence and a heavy chain variable sequence selected from clone-paired sequences of Table 2.

18. The method of claim 13, wherein said antibody or antibody fragment comprises a light chain variable sequence and a heavy chain variable sequence having 70%, 80%, or 90% identity to clone-paired sequences selected from Table 2.

19. The method of claim 13, wherein said antibody or antibody fragment comprises light and heavy chain variable sequences having 95% identity to clone-paired sequences from Table 2.

20. The method of claim 13, wherein the antibody fragment is a recombinant scFv (single chain fragment variable) antibody, Fab fragment, F(ab′)₂ fragment, or Fv fragment.

21. The method of claim 13, wherein said antibody is an IgG, or a recombinant IgG antibody or antibody fragment comprising a mutated Fc portion, such as to alter (eliminate or enhance) FcR interactions, to increase half-life and/or increase therapeutic efficacy, such as a LALA, N297, GASD/ALIE, YTE or LS mutation or glycan modified to alter (eliminate or enhance) FcR interactions such as enzymatic or chemical addition or removal of glycans or expression in a cell line engineered with a defined glycosylating pattern.

22. The method of claim 13, wherein said antibody is a chimeric antibody or a bispecific antibody.

23. The method of claim 13, wherein said antibody or antibody fragment is administered prior to infection or after infection.

24. The method of claim 13, wherein said subject is a pregnant female, a sexually active female, or a female undergoing fertility treatments.

25. The method of claim 13, wherein delivering comprises antibody or antibody fragment administration, or genetic delivery with an RNA or DNA sequence or vector encoding the antibody or antibody fragment.

26. A monoclonal antibody or fragment thereof, wherein the antibody or antibody fragment comprises clone-paired heavy and light chain CDR sequences, wherein the heavy chain CDR sequences are selected from Table 3, and the light chain CDR sequences are selected from Table 4.

27. The monoclonal antibody of claim 26, wherein said antibody or antibody fragment is encoded by light and heavy chain variable nucleotide sequences according to clone-paired sequences selected from Table 1.

28. The monoclonal antibody of claim 26, wherein said antibody or antibody fragment is encoded by light and heavy chain variable nucleotide sequences having at least 70%, 80%, or 90% identity to clone-paired sequences selected from Table 1.

29. The monoclonal antibody of claim 26, wherein said antibody or antibody fragment is encoded by light and heavy chain variable nucleotide sequences having at least 95% identity to clone-paired sequences selected from Table 1.

30. The monoclonal antibody of claim 26, wherein said antibody or antibody fragment comprises a light chain variable sequence and a heavy chain variable sequence selected from clone-paired sequences of Table 2.

31. The monoclonal antibody of claim 26, wherein said antibody or antibody fragment comprises a light chain variable sequence and a heavy chain variable sequence having 95% identity to clone-paired sequences selected from Table 2.

32. The monoclonal antibody of claim 26, wherein the antibody fragment is a recombinant scFv (single chain fragment variable) antibody, Fab fragment, F(ab′)₂ fragment, or Fv fragment.

33. The monoclonal antibody of claim 26, wherein said antibody is a chimeric antibody, or a bispecific antibody.

34. The monoclonal antibody of claim 26, wherein said antibody is an IgG, or a recombinant IgG antibody or antibody fragment comprising a mutated Fc portion, such as to alter (eliminate or enhance) FcR interactions, to increase half-life and/or increase therapeutic efficacy, such as a LALA, N297, GASD/ALIE, YTE or LS mutation or glycan modified to alter (eliminate or enhance) FcR interactions such as enzymatic or chemical addition or removal of glycans or expression in a cell line engineered with a defined glycosylating pattern.

35. The monoclonal antibody of claim 26, wherein said antibody or antibody fragment further comprises a cell penetrating peptide and/or is an intrabody.

36. A hybridoma or engineered cell encoding an antibody or antibody fragment, wherein the antibody or antibody fragment comprises clone-paired heavy and light chain CDR sequences, wherein the heavy chain CDR sequences are selected from Table 3, and the light chain CDR sequences are selected from Table 4.

37. The hybridoma or engineered cell of claim 36, wherein said antibody or antibody fragment is encoded by light and heavy chain variable nucleotide sequences according to clone-paired sequences selected from Table 1.

38. The hybridoma or engineered cell of claim 36, wherein said antibody or antibody fragment is encoded by light and heavy chain variable nucleotide sequences having at least 70%, 80%, or 90% identity to clone-paired sequences selected from Table 1.

39. The hybridoma or engineered cell of claim 36, wherein said antibody or antibody fragment is encoded by light and heavy chain variable nucleotide sequences having at least 95% identity to clone-paired sequences selected from Table 1.

40. The hybridoma or engineered cell of claim 36, wherein said antibody or antibody fragment comprises a light chain variable sequence and a heavy chain variable sequence selected from clone-paired sequences of Table 2.

41. The hybridoma or engineered cell of claim 36, wherein said antibody or antibody fragment is encoded by light and heavy chain variable sequences having at least 70%, 80%, or 90% identity to clone-paired variable sequences from Table 2.

42. The hybridoma or engineered cell of claim 36, wherein said antibody or antibody fragment comprises light and heavy chain variable sequences having 95% identity to clone-paired sequences from Table 2.

43. The hybridoma or engineered cell of claim 36, wherein the antibody fragment is a recombinant scFv (single chain fragment variable) antibody, Fab fragment, F(ab′)₂ fragment, or Fv fragment.

44. The hybridoma or engineered cell of claim 36, wherein said antibody is a chimeric antibody or a bispecific antibody.

45. The hybridoma or engineered cell of claim 36, wherein said antibody is an IgG, or a recombinant IgG antibody or antibody fragment comprising a mutated Fc portion, such as to alter (eliminate or enhance) FcR interactions, to increase half-life and/or increase therapeutic efficacy, such as a LALA, N297, GASD/ALIE, YTE or LS mutation or glycan modified to alter (eliminate or enhance) FcR interactions such as enzymatic or chemical addition or removal of glycans or expression in a cell line engineered with a defined glycosylating pattern.

46. The hybridoma or engineered cell of claim 36, wherein said antibody or antibody fragment further comprises a cell penetrating peptide and/or is an intrabody.

47. A vaccine formulation comprising one or more antibodies or antibody fragments, wherein the antibody or antibody fragment comprises clone-paired heavy and light chain CDR sequences, wherein the heavy chain CDR sequences are selected from Table 3, and the light chain CDR sequences are selected from Table 4.

48. The vaccine formulation of claim 47, wherein said antibody or antibody fragment is encoded by light and heavy chain variable nucleotide sequences according to clone-paired sequences selected from Table 1.

49. The vaccine formulation of claim 47, wherein said antibody or antibody fragment is encoded by light and heavy chain variable nucleotide sequences having at least 70%, 80%, or 90% identity to clone-paired sequences selected from Table 1.

50. The vaccine formulation of claim 47, wherein said antibody or antibody fragment is encoded by light and heavy chain variable nucleotide sequences having at least 95% identity to clone-paired sequences selected from Table 1.

51. The vaccine formulation of claim 47, wherein said antibody or antibody fragment comprises a light chain variable sequence and a heavy chain variable sequence selected from clone-paired sequences of Table 2.

52. The vaccine formulation of claim 47, wherein said antibody or antibody fragment comprises a light chain variable sequence and a heavy chain variable sequence having 95% identity to clone-paired sequences selected from Table 2.

53. The vaccine formulation of claim 47, wherein at least one of said antibody fragments is a recombinant scFv (single chain fragment variable) antibody, Fab fragment, F(ab′)₂ fragment, or Fv fragment.

54. The vaccine formulation of claim 47, wherein at least one of said antibodies is a chimeric antibody or a bispecific antibody.

55. The vaccine formulation of claim 47, wherein said antibody is an IgG, or a recombinant IgG antibody or antibody fragment comprising a mutated Fc portion, such as to alter (eliminate or enhance) FcR interactions, to increase half-life and/or increase therapeutic efficacy, such as a LALA, N297, GASD/ALIE, YTE or LS mutation or glycan modified to alter (eliminate or enhance) FcR interactions such as enzymatic or chemical addition or removal of glycans or expression in a cell line engineered with a defined glycosylating pattern.

56. The vaccine formulation of claim 47, wherein at least one of said antibodies or antibody fragments further comprises a cell penetrating peptide and/or is an intrabody.

57. A vaccine formulation comprising one or more expression vectors encoding a first antibody or antibody fragment according to claim 26.

58. The vaccine formulation of claim 57, wherein said expression vector(s) is/are Sindbis virus or VEE vector(s).

59. The vaccine formulation of claim 57, wherein the vaccine is formulated for delivery by needle injection, jet injection, or electroporation.

60. The vaccine formulation of claim 57, further comprising one or more expression vectors encoding for a second antibody or antibody fragment, such as a distinct antibody or antibody fragment, wherein the antibody or antibody fragment comprises clone-paired heavy and light chain CDR sequences, wherein the heavy chain CDR sequences are selected from Table 3, and the light chain CDR sequences are selected from Table 4.

61. A method of protecting the health of a placenta and/or fetus of a pregnant subject infected with or at risk of infection with Candida comprising delivering to said subject an antibody or antibody fragment wherein the antibody or antibody fragment comprises clone-paired heavy and light chain CDR sequences, wherein the heavy chain CDR sequences are selected from Table 3, and the light chain CDR sequences are selected from Table 4.

62. The method of claim 61, wherein said antibody or antibody fragment is encoded by light and heavy chain variable nucleotide sequences according to clone-paired sequences selected from Table 1.

63. The method of claim 61, wherein said antibody or antibody fragment is encoded by light and heavy chain variable nucleotide sequences having at least 70%, 80%, or 90% identity to clone-paired sequences selected from Table 1.

64. The method of claim 61, wherein said antibody or antibody fragment is encoded by light and heavy chain variable nucleotide sequences having at least 95% identity to clone-paired sequences selected from Table 1.

65. The method of claim 61, wherein said antibody or antibody fragment comprises a light chain variable sequence and a heavy chain variable sequence selected from clone-paired sequences of Table 2.

66. The method of claim 61, wherein said antibody or antibody fragment comprises light and heavy chain variable sequences having 70%, 80% or 90% identity to clone-paired sequences from Table 2.

67. The method of claim 61, wherein said antibody or antibody fragment comprises light and heavy chain variable sequences having 95% identity to clone-paired sequences from Table 2.

68. The method of claim 61, wherein the antibody fragment is a recombinant scFv (single chain fragment variable) antibody, Fab fragment, F(ab′)₂ fragment, or Fv fragment.

69. The method of claim 61, wherein said antibody is an IgG, or a recombinant IgG antibody or antibody fragment comprising a mutated Fc portion, such as to alter (eliminate or enhance) FcR interactions, to increase half-life and/or increase therapeutic efficacy, such as a LALA, N297, GASD/ALIE, YTE or LS mutation or glycan modified to alter (eliminate or enhance) FcR interactions such as enzymatic or chemical addition or removal of glycans or expression in a cell line engineered with a defined glycosylating pattern.

70. The method of claim 61, wherein said antibody is a chimeric antibody or a bispecific antibody.

71. The method of claim 61, wherein said antibody or antibody fragment is administered prior to infection or after infection.

72. The method of claim 61, wherein said subject is a pregnant female, a sexually active female, or a female undergoing fertility treatments.

73. The method of claim 61, wherein delivering comprises antibody or antibody fragment administration, or genetic delivery with an RNA or DNA sequence or vector encoding the antibody or antibody fragment.

74. The method of claim 61, wherein the antibody or antibody fragment increases the size of the placenta as compared to an untreated control.

75. The method of claim 61, wherein the antibody or antibody fragment reduces fungal load and/or pathology of the fetus as compared to an untreated control.

76. A method of determining the antigenic integrity, correct conformation and/or correct sequence of a Candida antigen comprising:
(a) contacting a sample comprising said antigen with a first antibody or antibody fragment wherein the antibody or antibody fragment comprises clone-paired heavy and light chain CDR sequences, wherein the heavy chain CDR sequences are selected from Table 3, and the light chain CDR sequences are selected from Table 4; and
(b) determining antigenic integrity, correct conformation and/or correct sequence of said antigen by detectable binding of said first antibody or antibody fragment to said antigen.

77. The method of claim 76, wherein said sample comprises recombinantly produced antigen.

78. The method of claim 76, wherein said sample comprises a vaccine formulation or vaccine production batch.

79. The method of claim 76, wherein detection comprises ELISA, RIA, western blot, a biosensor using surface plasmon resonance or biolayer interferometry, or flow cytometric staining.

80. The method of claim 76, wherein said antibody or antibody fragment is encoded by light and heavy chain variable nucleotide sequences according to clone-paired sequences selected from Table 1.

81. The method of claim 76, wherein said antibody or antibody fragment is encoded by light and heavy chain variable nucleotide sequences having at least 70%, 80%, or 90% identity to clone-paired sequences selected from Table 1.

82. The method of claim 76, wherein said antibody or antibody fragment is encoded by light and heavy chain variable nucleotide sequences having at least 95% identity to clone-paired sequences selected from Table 1.

83. The method of claim 76, wherein said antibody or antibody fragment comprises a light chain variable sequence and a heavy chain variable sequence selected from clone-paired sequences of Table 2.

84. The method of claim 76, wherein said first antibody or antibody fragment comprises light and heavy chain variable sequences having 70%, 80% or 90% identity to clone-paired sequences from Table 2.

85. The method of claim 76, wherein said first antibody or antibody fragment comprises light and heavy chain variable sequences having 95% identity to clone-paired sequences from Table 2.

86. The method of claim 76, wherein the first antibody fragment is a recombinant scFv (single chain fragment variable) antibody, Fab fragment, F(ab′)₂ fragment, or Fv fragment.

87. The method of claim 76, further comprising performing steps (a) and (b) a second time to determine the antigenic stability of the antigen over time.

88. The method of claim 76, further comprising:
(c) contacting a sample comprising said antigen with a second antibody or antibody fragment, wherein the antibody or antibody fragment comprises clone-paired heavy and light chain CDR sequences, wherein the heavy chain CDR sequences are selected from Table 3, and the light chain CDR sequences are selected from Table 4; and
(d) determining antigenic integrity of said antigen by detectable binding of said second antibody or antibody fragment to said antigen.

89. The method of claim 88, wherein said antibody or antibody fragment is encoded by light and heavy chain variable nucleotide sequences according to clone-paired sequences selected from Table 1.

90. The method of claim 88, wherein said antibody or antibody fragment is encoded by light and heavy chain variable nucleotide sequences having at least 70%, 80%, or 90% identity to clone-paired sequences selected from Table 1.

91. The method of claim 88, wherein said antibody or antibody fragment is encoded by light and heavy chain variable nucleotide sequences having at least 95% identity to clone-paired sequences selected from Table 1.

92. The method of claim 88, wherein said antibody or antibody fragment comprises a light chain variable sequence and a heavy chain variable sequence selected from clone-paired sequences of Table 2.

93. The method of claim 88, wherein said first antibody or antibody fragment comprises light and heavy chain variable sequences having 70%, 80% or 90% identity to clone-paired sequences from Table 2.

94. The method of claim 88, wherein said first antibody or antibody fragment comprises light and heavy chain variable sequences having 95% identity to clone-paired sequences from Table 2.

95. The method of claim 88, wherein the second antibody fragment is a recombinant scFv (single chain fragment variable) antibody, Fab fragment, F(ab′)₂ fragment, or Fv fragment.

96. The method of claim 88, further comprising performing steps (c) and (d) a second time to determine the antigenic stability of the antigen over time.

97. A pharmaceutical composition comprising the antibody or fragment thereof according to claim 26, and a pharmaceutically acceptable carrier or excipient.

98. The pharmaceutical composition of claim 97, further comprising at least one additional therapeutic agent.

99. The pharmaceutical composition of claim 98, wherein the therapeutic agent is a toxin, a radiolabel, a siRNA, a small molecule, or a cytokine.
